(19) United States
(12) Patent Application Publication
(10) Pub. No.: US 2017/0246267 A1
(43) Pub. Date: Aug. 31, 2017

(54) CHOLINERGIC ANTAGONISM AS AN ADJUNCT TO CANCER THERAPY

(71) Applicants: The Trustees of Columbia University in the City of New York, New York, NY (US); Norwegian University of Science and Technology, Trondheim (NO)

(72) Inventors: Timothy C. WANG, New York, NY (US); Duan CHEN, Trondheim (NO); Chun-Mei ZHAO, Trondheim (NO)

(21) Appl. No.: 15/437,948

(22) Filed: Feb. 21, 2017

Related U.S. Application Data
(63) Continuation-in-part of application No. PCT/US15/45639, filed on Aug. 18, 2015.
(60) Provisional application No. 62/443,251, filed on Jan. 6, 2017.
(60) Provisional application No. 62/038,629, filed on Aug. 18, 2014.

(51) Int. Cl. A61K 38/48; A61K 31/4178; A61K 31/325; A61B 17/00; A61K 31/513; A61K 31/555; A61K 31/4025; A61K 31/437; A61K 9/00

(52) U.S. Cl. CPC A61K 38/4893; A61K 31/437; A61K 31/4178; A61K 31/325; C12Y 304/24069; A61K 9/0019; A61K 31/513; A61K 31/555; A61K 9/0004; A61K 31/4025; A61B 17/00234; A61B 2017/00818; A61B 2017/00269

(57) ABSTRACT

The invention provides for methods for treating gastric cancer or colon cancer in a subject by administering a cholinergic antagonist, a Botulinum toxin, a NGF inhibitior, a TRK inhibitor, or performing a surgical denervation. The invention provides for inhibiting stem cells growth by administering a cholinergic antagonist, a Botulinum toxin, a NGF inhibitior, a TRK inhibitor, or performing a surgical denervation. The invention provides for stimulating regeneration of the colon or stomach by administering a cholinergic agonist.

This application is a continuation-in-part of International Application No. PCT/US15/45639, filed on Aug. 18, 2015, which claims the benefit of and priority to U.S. Ser. No. 62/038,629 filed Aug. 18, 2014, the contents of each of which is hereby incorporated by reference in their entireties. This application also claims the benefit of and priority to U.S. Ser. No. 62/443,251 filed Jan. 6, 2017, the contents of which is hereby incorporated by reference in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### GOVERNMENT SUPPORT

This invention was made with government support under Grant No. NIH 5R01 DK060758-10 awarded by the National Institute of Health and the National Institute of Diabetes and Digestive and Kidney Diseases. The Government has certain rights in the invention.

### BACKGROUND OF THE INVENTION

Gastric cancer is a severe and often fatal condition in the United States with more than 21,600 new cases reported annually, of which over 10,000 are expected to be fatal. Current treatments rely on a standard combination of chemotherapy, surgery, and radiation to slow tumor growth or if possible, remove the affected cells. Surgery either through endoscopic resection of the tumor or gastrectomy is the current gold standard of treatment. For certain patients, especially those with widespread disease or metastasis, chemotherapy and radiation are useful adjuvant treatments. While these treatments have improved survivability in patients with this disease, mortality remains as high as nearly 50%. There is a continuing need for novel treatments for cancers, including gastric cancers.

Proctitis, colitis, and other inflammatory bowel diseases, characterized by the inflammation of the tissue lining of the gastrointestinal (GI) tract, affects millions of people nationwide. These conditions are often painful and are a common side effect of radiation therapy and/or chemotherapy for the treatment of cancer. There is a continuing need for novel treatments for these conditions.

### SUMMARY OF THE INVENTION

As would be apparent to one of ordinary skill in the art, any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

In one aspect, the invention provides a method for treating cancer in a subject in need thereof, the method comprising administering to the subject a cholinergic antagonist, a Botulinum toxin, an acetylcholine (ACh) inhibitor, a nerve growth factor (NGF) inhibitor, a tropomyosin receptor kinase (TRK) inhibitor, or a combination thereof. In some embodiments, the method further comprises performing surgical denervation.

In one aspect, the invention provides a method for treating cancer in a subject in need thereof, the method comprising performing surgical denervation.

In one aspect, the invention provides a method for reducing proliferation of tumor cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for reducing proliferation of tumor cells, the method comprising performing surgical denervation.

In one aspect, the invention provides a method for inhibiting proliferation of tumor cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for inhibiting proliferation of tumor cells, the method comprising performing surgical denervation.

In one aspect, the invention provides a method for inhibiting tumor metastasis, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for inhibiting tumor metastasis, the method comprising performing surgical denervation.

In one aspect, the invention provides a method for treating tumor reoccurrence in a subject in need thereof, the method comprising administering to the subject a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for treating tumor reoccurrence in a subject in need thereof, the method comprising performing surgical denervation.

In one aspect, the invention provides a method for inhibiting stem cell growth, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for inhibiting stem cell growth, the method comprising performing surgical denervation.

In one aspect, the invention provides a method for inhibiting Wnt signaling in stem cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In one aspect, the invention provides a method for inhibiting Wnt signaling in stem cells, the method comprising performing surgical denervation.

In one aspect, the invention provides a method for stimulating growth of stem cells, the method comprising administering a cholinergic agonist.

In some embodiments, the surgical denervation is a vagotomy. In some embodiments, the surgical denervation is a bilateral vagotomy with pyloroplasty. In some embodiments, the surgical denervation is a unilateral vagotomy. In some embodiments, the Botulinum toxin inhibits local signaling from the vagus nerve. In some embodiments the cholinergic antagonist is a small molecule. In some embodiments, the cholinergic antagonist is a muscarinic receptor antagonist. In some embodiments, the cholinergic antagonist is a M3 receptor antagonist. In some embodiments, the cholinergic antagonist is darifenacin, scopolamine, amitriptyline, or a tricyclic antidepressant. In some embodiments, the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486 . In some embodiments, the TRK inhibitor is an anti-TRK antibody.

In some embodiments, the method further comprises administering a cytotoxic therapy. In some embodiments, the cholinergic antagonist, Botulinum toxin, ACh inhibitor, NGF inhibitor, TRK inhibitor, or surgical denervation is administered or performed before, during, or after the administration of the cytotoxic therapy. In some embodiments, the cytotoxic therapy is radiotherapy or chemotherapy.

In some embodiments, the method further comprises performing an endoscopic resection surgery or a gastrectomy surgery. In some embodiments, the cholinergic antagonist, Botulinum toxin, ACh inhibitor, NGF inhibitor, TRK inhibitor, or surgical denervation is administered or performed before, during, or after the endoscopic resection surgery or gastrectomy surgery is performed.

In some embodiments, the cancer is gastric cancer. In some embodiments, the cancer is colon cancer. In some embodiments, the tumor is a gastric tumor. In some embodiments, the tumor is a colon tumor. In some embodiments, the stem cells are cancer stem cells. In some embodiments, the cancer stem cells are gastric cancer stem cells. In some embodiments, the cancer stem cells are colon cancer stem cells. In some embodiments, the stem cells express Lgr5. In some embodiments, the stem cells express M3 receptor. In some embodiments, the stem cells are gastric stem cells.

### BRIEF DESCRIPTION OF THE FIGURES

The patent or application file contains at least one drawing executed in color. Copies of this patent or patent application publication with color drawing(s) will be provided by the United States Patent and Trademark Office upon request and payment of the necessary fee. The original color version of some of the Figures can be viewed in Zhao, et al. (2014), Denervation suppresses gastric tumorigenesis, Sci. Transl. Med., Vol. 6, 250ra115 (including the accompanying Supplementary Information) and in Hayakawa et al., (2017), Nerve Growth Factor Promotes Gastric Tumorigenesis through Aberrant Cholinergic Signaling, Cancer Cell, Vol. 31(1), 21-24 (including the accompanying Supplementary Information). The contents of Zhao et al. (2014) and Hayakawa et al. (2017), including the accompanying “Supplementary Information,” are hereby incorporated by reference in their entireties.

FIGS. 1A-1N. Denervation attenuates tumorigenesis at the preneoplastic stage in mouse models of gastric cancer. (A) Tumor prevalence at the lesser curvature (LC) and greater curvature (GC) of the stomach of INS-GAS mice. (B) Images of carbocyanine dye (DiI)—labeled vagal terminals in an adult mouse stomach. A montage of a low-power image showing the lesser curvature and greater curvature of the gastric wall (top scale bar, 2.0 mm), and higher-power images of lesser curvature (middle) and greater curvature (bottom) show a higher density of vagal innervation in lesser curvature than in greater curvature (89 and 54% of the visual field, estimated by point counting method) (middle and bottom scale bars, 72 μm). (C) Tumor incidence at 12 months of age in INSGAS mice that underwent sham operation (Sham), pyloroplasty alone (PP), bilateral vagotomy with pyloroplasty (VTPP), or anterior unilateral vagotomy (UVT) (A, anterior; P, posterior side of the stomachs) at 6 months of age. ***P=4.9×10⁻⁷ (VTPP versus PP), P=1.32×10⁻⁶ [UVT(A) versus UVT(P)] (Fisher's exact test). (D) Representative microphotographs of histopathological appearance of the anterior and posterior sides of the stomach from INS-GASmice (at 12 months of age) that underwent sham, PP, VTPP, and UVT at 6 months of age. Scale bars, 100 μm. (E) Pathological score for dysplasia. Means±SEM. Comparisons between anterior and posterior sides were performed by paired t test within sham (n=27) and UVT (n=30), or by Tukey test between PP (n=25) and VTPP (n=25). ***P=5.31×10−5 (UVT), P=0.0001 or 0.00006 (PP and VTPP, anterior or posterior side, respectively). ns, not significant (P=0.987). (F) Number of proliferating cells. Means±SEM. Comparisons between anterior and posterior sides were performed by paired t test within sham (n=27) and UVT (n=30), or by Tukey test between PP (n=25) and VTPP (n=25). ***P=5.77×10⁻³ (UVT), P=1.90×10⁻⁴ (anterior), and P=1.49×10⁻³ (posterior) between PP and VTPP. ns, not significant (P=0.229). (G) Representative photographs showing gross appearance of stomachs opened along the greater curvature and corresponding microphotographs of histopathological appearance of the stomachs (antrum) from mice treated with MNU+PP or MNU+VTPP. Scale bars, 100 μm. (H) Volume density of tumor (measured by point counting method). Means±SEM. Student's t test was used to compare between MNU+PP (n=11) and MNU+VTPP (n=9). (I) Representative photograph showing gross appearance of gastric tumors (indicated by dashed line) in a stomach opened along the greater curvature from an Hp-infected H¹/K¹−ATPase-IL-1β mouse, which underwent UVT in the anterior side (indicated by asterisk). (J) Number of proliferating cells in Hp-infected H⁺/K⁺−ATPase-IL-1β mouse stomachs subjected to UVT in the anterior side. Means±SEM. ***P=0.00006 (Student's t test). ns, not significant (P=0.120) between sham (n=12) and UVT (n=12) in the anterior and the posterior sides. (K) Photographs showing the Botox injection procedure (upper left), gross appearance of Botox-injected stomach after 6 months (A, anterior where Botox was injected; P, posterior) (upper right), and representative microphotographs of histopathological appearance of anterior (lower left) and posterior (lower right) stomach (corpus). Arrow, injection site. Scale bars, 100 μm. (L to N) Volume density of tumor, pathological score for dysplasia, and number of proliferating cells after anterior Botox injection. Means±SEM (n=16). ***P=2.75 ×10⁻¹¹ (L), P=1.00×10⁻² (M), or P=0.001 (N) between the anterior and posterior sides of the stomach (paired t test).

FIGS. 2A-2H. Denervation attenuates gastric tumor progression in mice. (A) Gross appearance of mouse stomachs at 18 months of age and representative microphotographs of the histopathological appearance of the corpus region of the anterior and posterior sides of the stomach from age-matched INS-GAS mice (Control) and mice that underwent anterior UVT at 8 or 12 months of age. Scale bars, 100 μm. Arrows, vagotomy side. (B) Volume density of tumor. Means±SEM. Paired t test between the anterior and the posterior sides of the stomach: P=0.589 (n=21, Control), P=2.56×10⁻⁵ (n=17, UVT at 8 months of age), P=2.17×10⁻⁴ (n=14, UVT at 10 months), P=0.055 (n=12,UVT at 12 months). (C) Pathological score for dysplasia. Means±SEM. Paired t test between the anterior and the posterior sides of the stomach: P=0.38 (n=21, Control), P=0.002 (n=17, UVT at 8 months of age), P=0.047 (n=14,UVT at 10 months), P=0.018 (n=12, UVT at 12 months). (D) Kaplan-Meier curves showing survival of INS-GAS mice that underwent UVT at 8 (grey line with squares), 10 (grey line with triangles), or 12 months of age (grey line with circles), or of age-matched INS-GAS mice (Control) (black line). P=0.01 between control and UVT groups at 8 months. (E) Proliferating cells in the anterior and posterior mucosa of the stomach of INS-GAS mice at 2 months after vagotomy and/or Botox injection. Means±SEM. Paired t test was used to compare the anterior and posterior sides of the stomach. P=0.291(n=6, Vehicle), P=0.007 [n=6, unilateral anterior Botox (UB)], P=0.595 [n=7, bilateral Botox (BB)], P=0.326 [n=7, bilateral Botox plus anterior UVT (BB+UVT)], P=0.0007 (Vehicle anterior versus UB anterior, Dunnett's test). (F) Volume density of tumor in INS-GAS mice subjected to saline (intraperitoneally) (Control), UB+saline (intraperitoneally), UB+5-fluorouracil (5-FU)+oxaliplatin (OXP) (intraperitoneally), or UVT+5-FU+OXP (intraperitoneally). Means±SEM. Paired t test was used to compare the anterior and posterior sides of the stomach. P=0.172 (n=10, Control), P=0.200 (n=10, UB+saline), P=0.0004 (n=24, UB+5-FU+OXP), P=0.006 (n=16, UVT+5-FU+OXP). (G) Gross appearance of representative stomachs from INS-GAS mice subjected to 5-FU+OXP with UB or UVT (reduced tumor burden indicated by arrows). (H) Kaplan-Meier curves showing survival of INS-GAS mice that underwent sham operation and 5-FU+OXP treatment (black), UB and 5-FU+OXP (grey line with squares), or anterior UVT and 5-FU+OXP (grey line with circles). *P=0.041, **P=0.0069.

FIG. 3. Denervation leads to inhibition of Wnt signaling in the mouse model of gastric cancer. Gene expression of Wnt signaling pathway (determined by qRT-PCR array analysis) in vagotomized anterior stomach of INS-GAS mice at 12 months of age (6 months UVT). Log₂ fold changes of expressed genes in comparison with the posterior side of the same stomach are shown. Left side, down-regulation; right-side, up-regulation.

FIGS. 4A-4F. Denervation alters inflammation related signaling and suppresses stem cell expansion in mouse models of gastric cancer. (A) Time course of five signaling pathways determined by microarray analysis in the anterior side of the stomach at 2 (grey line with squares), 4 (grey line with circles), and 6 (grey line with triangles) months after anterior UVT compared with the posterior side of the stomach in INSGAS mice. Total net accumulated perturbation (expressed as tA score): −4 to 6. tA score>0: activation; tA score<0: inhibition. (B) Numbers of CD44⁺ cells in the anterior and the posterior sides of the stomach of INS-GAS mice at 6 months after surgery. Means±SEM. P=0.037 (n=27, paired t test) between the anterior and the posterior sides in sham operation (Sham), P=1.00×10⁻⁶ or P=6.00×10⁻⁶ (n=25, Dunnett's test) between PP and VTPP (anterior and posterior sides, respectively), and P=1.74×10³ (n=30, paired t test) between the anterior and the posterior sides within anterior UVT. (C) Numbers of CD44-immunoreactive cells (CD44) and CD44v6-immunoreactive cells (CD44v6) in the anterior and the posterior sides of the stomach of INSGAS mice at 6 months after Botox injection. Means±SEM. P=0.034 and P=0.021, respectively (n=16, paired t test) between the anterior and the posterior sides of the stomach. (D) Relative gene expression of Cyclin D1, Axin2, Myc, Lgr5, and Cd44 in the gastric tumors of sham-operated or VTPP-treated mice 36 weeks after MNU treatment (n=4 per group). Means±SEM. **P=0.04 (Cyclin D1), 0.04 (Axin2), 0.03 (Myc), 0.001 (Lgr5), and 0.01 (Cd44) (Student's t test). (E) Number of cells showing nuclear β-catenin accumulation in the gastric tumors of PP- or VTPP-treated mice 36 weeks after MNU treatment (n=4 per group). Means±SEM. P=7.00×10⁻⁶ (Student's t test). Representative immunohistochemical microphotographs are shown below. Scale bars, 40 μm. (F) Number of Lgr5⁺ cells in the stomachs of PP- or VTPP-treated mice 6 weeks after MNU treatment (n=5 per group). Means±SEM. P=4.00×10⁻⁶ (Student's t test). Representative Lgr5-GFP¹ microphotographs are shown below. Scale bars, 20 μm.

FIGS. 5A-5F. M3 receptor signaling in gastric stem cells regulates tumorigenesis in mouse models of gastric cancer. (A) Representative fluorescence-activated cell sorting gating showing forward scatter (FSC) and Lgr5-GFP expression. (B and C) Relative gene expression of Lgr5 and muscarinic receptors (Chrm1 to Chrm5) in sorted Lgr5-negative, Lgr5-low, and Lgr5-high populations. Means±SEM (n=4). (D) Number of proliferating cells in the tumors of INS-GAS mice treated with saline (Control, n=19), M3 receptor antagonist darifenacin (M3R, n=15), 5-FU+oxaliplatin (Chemo, n=12), or combination of 5-FU+oxaliplatin+darifenacin (M3R+Chemo, n=8), respectively. Means±SEM. P values were calculated by Dunnett's test. (E) Representative photographs showing gross appearance of stomachs opened along the greater curvature from wild-type (WT) or M3 receptor knockout mice (M3KO) treated with MNU. (F) Volume density of tumor in the stomachs of MNU-treated WT (n=13) versus MNU-treated M3KO mice (n=7). Means±SEM (Student's t test).

FIGS. 6A-6H. Neurons activate Wnt signaling in gastric stem cells through the M3 receptor. (A to C) Representative microphotographs showing gastric organoids along with neurite outgrowth. (A) Guinea pig enteric neuron (arrowhead) with gastric organoids (asterisks). Scale bar, 5 μm. (B) Three-dimensional images (low and high magnifications) obtained by two-photon microscopy of gastric organoids (dark grey) derived from an ACTB-tDTomato mouse and neurons (light grey) derived from a UBC-GFP mouse. Scale bars, 20 μm (left) and 5 μm (right). In (C), fluorescent images show gastric organoids alone (left, Control) or co-cultured with neurons (right, Neuron) at day 4. Scale bars, 10 μm. (D) Relative number of organoids after 72 hours in control, neuron co-culture, control plus Botox, or neuron co-culture plus Botox. Means±SEM. n=4 per group. ***P=0.002 (Student's t test). ns, not significant compared to control. (E) Relative number of organoids after 72 hours in control or neuron co-culture with or without scopolamine (SCOP) (1 mg/ml). Means±SEM. n=4 per group. **P=0.003 (Student's t test). ns, not significant compared to control. (F) Relative number of organoids at day 10 with or without 100 μM pilocarpine. Means±SEM. n=4 per group. **P=0.006 (Student's t test) between control and pilocarpine. (G) Relative mRNA expression for Lgr5, Cd44, and Sox9 in relation to Gapdh on day 7 with or without 10 or 100 μM pilocarpine in gastric organoids isolated from WT or M3KO mice. Means±SEM. Student's t test between 0 μM and 10 or 100 μM pilocarpine. n=4 per group. (H) Relative number of organoids at day 10 with or without neurons and/or Wnt3a. Means±SEM. n=4 per group. ns, not significant. *P=0.030 compared to Control+Wnt3a (Student's t test).

FIG. 7. Gastric cancer patients exhibit a dysregulation of Wnt signaling. Gene expression of Wnt signaling pathway (microarray analysis) in human gastric cancer tissue. The graph shows log2 fold changes of expressed genes in comparison with the adjacent noncancerous tissue of the same stomach. Left-side, down-regulation; right-side, up-regulation.

FIGS. 8A-8F. PGP9.5 and peripherin may represent neural markers for gastric cancer progression. (A) Representative microphotographs showing human gastric cancer [indicated by arrowheads, hematoxylin and eosin (H&E) staining] and PGP9.5-labeled nerve (arrowhead). Scale bars, 50 μm. (B) Volume density of PGP9.5-labeled nerves in different levels of depth of tumor invasion [T2 (tumor invading muscularis propria) versus T3 (tumor penetrating subserosal connective tissue without invasion of visceral peritoneum or adjacent structures)] in the stage II and III gastric cancer patients. Means±SEM (n=120). P=0.008 (Student's t test). (C) Number of lymph node metastases in patients with stage II and III or stage IV gastric cancer that has low or high expression of PGP9.5. Means±SEM (n=120). P values were calculated by Student's t test. (D) PGP9.5- and peripherin-immunoreactive nerve densities in gastric mucosa of control mice (nontumor) and MNU-treated mice (tumor). PGP9.5 is a ubiquitin-protein hydrolase that is expressed in the neuronal cell bodies and axons in the central and peripheral nervous system. Peripherin is a type III intermediate filament protein that is expressed in peripheral and some central nervous system neurons. Both can be used as neuronal markers in the gut. Means±SEM (n=6 per group). P values were calculated by Student's t test. (E and F) Representative immunohistochemical microphotographs showing PGP9.5 and peripherin (indicated by arrows) in the nontumor and tumor areas of the mouse stomachs. Scale bars, 20 μm (E) and 40 μm (F).

FIGS. 9A-9G. Flowchart showing the animal study design.

FIG. 10. Anterior unilateral truncal vagotomy in mice. Photographs showing dissected vagus nerve (indicated by arrows) before and after anterior unilateral truncal vagotomy (UVT) are shown.

FIG. 11. Body weight of male and female mice after surgery in INS-GAS mice. Body weight of sham (Sham), pyloroplasty (PP), bilateral vagotomy with pyloroplasty (VTPP) and anterior unilateral vagotomy (UVT)-operated INS-GAS mice at 12 months of age (6 months postoperatively). Means±SEM. ns: not significant between Sham and PP, VTPP or UVT.

FIG. 12. Thickness of the gastric oxyntic mucosa after surgery in INS-GAS mice. Thickness of the oxyntic mucosa in sham (Sham), pyloroplasty (PP), bilateral vagotomy with pyloroplasty (VTPP) and anterior unilateral vagotomy (UVT)-operated INS-GAS mice at 12 months of age (6 months postoperatively). Means±SEM. Comparisons between anterior vs. posterior sides by paired t test within Sham (N=27) and UVT (N=30) or by Tukey test between PP (N=25) and VTPP (N=25).

FIGS. 13A-13F. Pathological scores for the stomach after surgery in INS-GAS mice. Pathological scores of inflammation (A), epithelial defects (B), oxyntic atrophy (C), epithelial hyperplasia (D), pseudopyloric metaplasia (E) and GHAI (gastric histological activity index) (F) in sham (Sham), pyloroplasty (PP), bilateral vagotomy with pyloroplasty (VTPP) and anterior unilateral vagotomy (UVT)-operated INS-GAS mice at 12 months of age (at 6 months after surgery). Means±SEM. Comparisons between anterior vs. posterior sides by paired t test within Sham (N=27) and UVT (N=30) or by Tukey test between PP (N=25) and VTPP (N=25).

FIGS. 14A-14B. Pathological scores for the stomach after Botox® injection in INS-GAS mice. Pathological scores of inflammation, epithelial defects, oxyntic atrophy, epithelial hyperplasia, pseudopyloric metaplasia (A) and GHAI (gastric histological activity index) (B) in Botox injected (in anterior side of the stomach) INS-GAS mice at 12 months of age (monthly Botox injection starting at 6 months of age). Means±SEM (N=16). Paired t test between anterior vs. posterior sides.

FIG. 15. Wnt signaling in INS-GAS mice compared with WT mice. Fold changes of Wnt-related genes: unregulated (indicated by left-side) and downregulated (right-side).

FIG. 16. Altered signaling pathways after vagotomy in INS-GAS mice. Altered signaling pathways involved gastric acid, MAPK signaling and tissue homeostasis at 2 (diamonds), 4 (triangle) and 6 (square) months in the anterior oxyntic mucosa of the stomach after anterior unilateral vagotomy compared with posterior side. tA score: −4 to 6. tA score>0: activation; tA score<0: inhibition.

FIG. 17. Wnt and Notch signaling pathways after vagotomy in INS-GAS mice. Wnt and Notch signaling KEGG pathways of the anterior oxyntic mucosa of the stomach after anterior unilateral vagotomy compared with the posterior side at 6 months. Down-regulated genes (p<0.05, indicated by pink); Unchanged genes (lighter grey).

FIG. 18. Immunostaining of CD44 after vagotomy in INS-GAS mice. Representative microphotographs of CD44+ cells in oxyntic mucosa of the anterior and the posterior regions of the same mouse stomach subjected to unilateral anterior vagotomy (UVT). Bars=25 μm.

FIGS. 19A-19B. Numbers of CD44 immunoreactive cells after Botox® treatment +/− vagotomy in INS-GAS mice. CD44 immunoreactive cells in mice subjected to saline (control) or Botox injection into anterior and posterior sides of the stomach (A) and Botox injection into anterior and posterior sides plus anterior UVT (B). Means±SEM. Student's t test between control (N=6) and Botox (N=7).

FIGS. 20A-20B. Tumor regeneration in the stomach after vagotomy in INS-GAS mice. (A) Microphotographs showing histopathological appearances of tumor regeneration at 3 weeks after acetic acid-induced necrotic ulcer. Note: regeneration (indicated by arrow) without unilateral vagotomy (No UVT), and no regeneration (arrow) after UVT. Bars: 50 μm. (B) Volume density of tumor regeneration 1, 2 and 3 weeks after application of acetic acid in mice with or without UVT. Means±SEM. Student's t test between no UVT and UVT (N=6, except N=8 or 5, respectively at 3 weeks).

FIG. 21. Wnt signaling pathway in M3KO mice. Wnt signaling KEGG pathway in M₃ receptor knockout mice in comparison with wild-type mice. Downregulated genes (p<0.05, indicated by light grey text on light grey box); Unchanged genes (black text on grey box).

FIG. 22. Altered expressed signaling pathways in human gastric cancer tissue. Altered signaling pathways in human gastric cancer tissue compared with adjacent non-cancerous tissue. tA score>0: activation (indicated by right-side); tA score<0: inhibition (left-side).

FIGS. 23A-23B. Gastric stump cancer after distal gastrectomy with or without vagotomy. (A) Tumors in both anterior and posterior walls in 24 patients without vagotomy. (B) None in anterior and one tumor in posterior walls in 13 patients with vagotomy (p<0.05). p=0.01898 or p=0.02718 in anterior or posterior wall of vagotomized patients compared with non-vagotomized patients (Fisher test).

FIGS. 24A-24B. Effect of 5-FU and oxaliplatin on INS-GAS mice tumor. Tumor size in anterior (A) or posterior (B) sides of stomachs in mice treated with saline (Control, N=10), 5-FU (N=10), or oxaliplatin (OXP, N=13), respectively. Means±SEM. Dunnett's test.

FIG. 25. Schematic of tumor-induced neurogenesis. Tumors secreted neurotrophins that lead to growth of nerve axons towards the tumor. Nerves then secrete neurotransmitters and/or growth factors that stimulate proliferation of tumors.

FIG. 26. Schematic of the generation of conditional NGF overexpression mice. The construct CAG-LSL-NGF-IRES-EGFP was generated through recombineering of the Rosa26 BAC and then inserted into the mouse germline. Expression of NGF and EGFP is prevented by a stop codon surrounded by loxP sites. By mating with Cre-expressing transgenic mice, the Stop sequence is removed and NGF will be expressed in specific cell types.

FIG. 27. Gastric epithelium-specific Cre-expressing mice. The TFF2-Cre (line3a) was crossed to Rosa26-mTmG (Tomato-GFP). Cre recombination leads to loss of tomato (red) expression and activation of GFP expression. This image of the mouse stomach shows entire gastric glands in the fundus (left) and antrum (right) are GFP positive. Thus, TFF2-Cre targets all gastric epithelial cells.

FIGS. 28A-28E. NGF is upregulated in gastric cancers. (A) Left. Relative expression per Gapdh of neurotrophin family (NGF, BDNF, NT3, NT4, and GDNF) in MNU-treated mouse non-tumor and tumor tissue. Right: In situ hybridization of NGF in MNU-treated non-tumor/tumor area. (B) FACS plot of EpCAM and CD45 from MNU tumors. Epithelial cells and immune cells are sorted separately. (C) Relative expression per Gapdh of NGF in EpCAM+cells and CD45+cells isolated from WT and MNU mice. NGF is specifically expressed in MNU-treated epithelial cells. (D) IHC of NGF in MNU-treated mice and human gastric cancer patients. NGF is expressed in cancer cells. (E) (Left.) NGF gene expression in cultured gastric organoid from MNU tumor. NGF expression is decreased at day 7 compared to day 1, but is upregulated by treatment with carbachol. (Middle) NGF expression in cultured gastric organoids from WT and M3 Receptor Knockout (M3RKO) mice. NGF is upregulated by carbachol treatment, but shows no change in M3RKO organoids. (Right) NGF expression in MNU tumor with or without vagotomy. Vagotomy downregulated NGF expression in tumors.

FIGS. 29A-29G. NGF/Trk signaling regulates mucosal innervation and proliferation. (A) Gene construct of R26-LSL-NGF-IRES-EGFP mice. (B) H&E staining of WT stomach and Tff2-Cre; R26-NGF mice. NGF overexpression leads to abnormal gland structure. (C) NGF expression in WT and Tff2-Cre; R26-NGF mice. (D) Neuron and glial marker staining (peripherin, Gap43(neuron), GFAP (glia), TH (sympathetic nerves), and VachT (cholinergic nerves) in WT and Tff2-Cre; R26-NGF mice. (E) shows quantification of positive staining in WT and TFF2-Cre mice. (F). Ki67 staining showing gastric mucosal proliferation in control (WT) mice compared to TFF2-Cre; R26-LSL-NGF (or NGF) mice, compared to TFF2-Cre;R26-LSL-NGF;M3R F/F mice. The latter mice also have a conditional knockout of the muscarinic-3 (M3) receptor and thus no longer responded to cholinergic stimulation driven by NGF overexpression. NGF overexpression promotes proliferation but it is blocked by M3R knockout. (G). Effect of TRK inhibition on NGF-dependent gastric proliferation and dysplasia. TFF2-Cre;R26-LSL-NGF mice were put on a normal diet up until 3 months of age, and then treated with the TRK inhibitor (PLX7486) in their mouse chow for one month, and then put on a normal diet again. Gastric sections were stained for Ki67 (top) or for peripherin (bottom). TRK inhibition completely reversed the neurogenesis and proliferation due to NGF overexpression in the stomach, but the inhibitory effect was reversible and lost when the mice resumed a normal diet.

FIG. 30. NGF overexpression in the small intestine and colon leads to marked innervation. H&E (top) and peripherin staining (bottom) in small intestine and colon from WT and Villin-Cre; R26-LSL-NGF mice. Increased innervation, as manifested by red peripherin staining, is seen in the NGF-overexpressing mouse intestine and colon.

FIGS. 31A-31K. Abnormal cholinergic innervation promotes gastric proliferation and carcinogenesis. (A) H&E, Ki67, CD44, and Alcian blue staining of 8 mo old Tff2-Cre; R26-NGF mice. These mice develop spontaneous dysplasia. (B) and (C) H&E and Ki67 staining of Tff2-Cre; R26-NGF and Tff2-Cre; R26-NGF; M3RF/F mice. (D) Incidence of dysplasia in stomach and duodenum in WT, Tff2-Cre; R26-NGF and Tff2-Cre; R26-NGF; M3RF/F mice. M3R knockout blocks the development of dysplasia. (E)-(G) MNU treatment of WT, Tff2-Cre; R26-NGF, and Tff2-Cre; R26-NGF; M3RF/F mice. NGF overexpression promotes MNU induced tumor development with a more aggressive histopathology. M3R knockout blocks this effect. (H) and (I) PLX7486 treatment in the MNU mouse model of stomach cancer. PLX7486 prevented tumor growth in both WT and Tff2-Cre; R26-NGF mice. (J) and (K) Gap43 (red), CD44 (green) staining and beta-catenin staining (brown) in MNU tumor with or without PLX7486 treatment. PLX7486 decreased nerve density, the number of CD44+ cells, and the number of beta-catenin+ cells.

FIG. 32. Villin-Cre; NGF mice develop colonic dysplasia. H&E staining (top) and Ki67 staining (bottom) of colonic sections from Villin-Cre; R26-NGF mice which show colonic dysplasia at 6 months.

FIG. 33. Villin-Cre; NGF mice are more susceptible to AOM-DSS tumor. Gross specimens from control and Villin-Cre; R26-LSL-NGF mice after treatment with AOM-DSS (top). Bottom shows quantification of tumor number and tumor size. In the AOMDSS colon cancer model, villinCre; NGF mice show increased number and size of colonic tumors.

FIGS. 34A-34C. Source of Ach in gut. (A) and (B) show staining for ChaT-GFP (green) expression with S100B (red) or peripherin (red) staining in the antrum and intestine. ChaT (choline acetyltransferase, which produces acetylcholine) is expressed in enteric ganglia, mucosal nerve fibers, and scattered rare epithelial cells. (C) Dclk1 staining (red) in ChaT-GFP (green) mice. ChaT+ epithelial cells are Dclk1+ tuft cells. Dclk1 is also expressed in ChaT+ ganglia and nerves, suggesting that Dclk1 +cells mark the source of Ach in the gut.

FIG. 35. Ablation of Dclk1+cells in Dclk1-CreERT; R26-DTA mice. (Top) shows Ki67 staining of WT or Dclk1-DTA mice on water or with bethanechol treatment. (Bottom) shows the treatment regimen, quantification of Ki67+ cells, and a schematic of the location of Dclk1 +tuft cells and Dclk1+nerves relative to Lgr5+stem cells. Ablation of Dclk1+cells leads to a decrease of proliferation in the stomach. Treatment with bethanechol can rescue this effect at least in part. Dclk1+tuft cells and nerves seem to regulate mucosal homeostasis through cholinergic signal, possibly acting on Lgr5+ stem cells expressing the M3 receptor.

FIG. 36. Villin-NGF mice are resistant to colonic injury. The role of NGF overexpression in injury responses. (Left) shows changes in body weight with 2.5% DSS in the drinking water in control versus Villin-Cre;R26-LSL-NGF mice. There is a significant difference in body weight at day 10 (p<0.05). (Right) Ki67 staining of control versus Villin-Cre;NGF mice. Villin-Cre;NGF mice are more resistant to DSS-colitis compared to control mice, and they show less weight loss and have more proliferating cells in the mucosa.

FIG. 37. Cholinergic signaling promotes mucosal regeneration and healing in colitis. DSS-colitis on day 7. (Top) H&E stained sections of WT, Villin-Cre; M3R F/F and Villin-Cre; R26-LSL-NGF; M3R F/F mice. (Bottom) Ki67 of WT mice treated with DSS with or without bethanechol in the drinking water. Deletion of M3R in colonic mucosa leads to more severe colitis, even in NGF overexpression mice. Treatment with bethanechol increased proliferation in DSS-treated WT mice. Ach/M3R signaling promotes colonic regeneration after DSS.

FIG. 38. Acetylcholine from Dclk1 + tuft cells and nerves induces NGF in gastric epithelial cells, which promotes neuron expansion and tumorigenesis. YAP is activated through the cholinergic signaling, and inhibition of this pathway can block NGF-driven tumors.

FIGS. 39A-E. ChAT⁺ Tuft Cells and Nerves Expand during Carcinogenesis. (A) Dclk1 staining (red) in Chat-GFP (green) mice antrum. Blue arrow indicates tuft cells, and yellow arrow indicates nerves. (B) Left, Peripherin staining (red) in Chat-GFP mice antrum. Arrows indicate GFP⁺ nerves. Right, Dclk1 staining (red) in Lgr5-GFP mice antrum. (C) Chat-GFP expression with or without MNU treatment (3 and 9 months after the beginning of MNU). (D) The number of ChAT⁺ epithelial cells per gland in MNU-treated or untreated stomachs. A total of 100 glands per group were analyzed. (E) Cell position of ChAT⁺ stromal cells in MNU-treated or untreated stomachs. A total of 50 glands per group was analyzed. Means±SEM. *p<0.05 (ANOVA). DAPI, blue. Scale bars, 20 mm. See also FIGS. 46A-F.

FIGS. 40A-F. ACh Signaling Stimulates NGF Production. (A) Ngf expression in cultured gastric organoids from WT and Chrm3 knockout (M3R KO) mice. Organoids were treated with carbachol at a concentration of 0, 10, or 100 mM for 7 days. n=4/group. GAP43, growth-associated protein 43; GFAP, glial fibrillary acidic protein. (B) Relative expression per Gapdh of neurotrophin family in MNU-treated mouse non-tumor and tumor tissues. The average expression of each gene in non-tumor tissues is set as 1.0. n=4/group. ND, not detected. (C) Relative Ngf expression per Gapdh in MNU tumors isolated from mice which have taken vagotomy or sham treatment. n=3/group. (D) In situ hybridization of Ngf in MNU-treated non-tumor and tumor areas. (E) Fluorescence-activated cell sorting plot of EpCAM and CD45 from MNU tumors. (F) Relative Ngf expression per Gapdh in EpCAM cells and CD45⁺ cells isolated from WT and MNU-treated mice. n=3/group. Means±SEM. *p<0.05; ANOVA in (A), t test in (B), (C), and (F). Scale bars, 20 mm. See also FIGS. 47A-C.

FIGS. 41A-J. NGF/Trk Signaling Regulates Mucosal Innervation (A) Gene construct of R26-LSL-Ngf-IRES-EGFP mice.(B) H&E staining of R26-NGF and Tff2-Cre; R26-NGF mouse stomach. (C and D) Neuron and glial marker staining (C) (red) and quantification (D) in R26-NGF and Tff2-Cre; R26-NGF (green) mice. The percentages of positive area per total mucosal area are quantified in four images per group. (E and F) Nes-GFP expression (E) and the cell number of GFP⁺ cells (F) in lamina propria (per stromal gap between two glands) and ganglia (per ganglia) in 6-week-old R26-NGF; Nes-GFP and Tff2-Cre; R26-NGF; R26-TdTomato; Nes-GFP mice. A total 20 glands and 20 ganglia was analyzed. Both NGF⁺ Tff2-Cre lineage cells and Nes-GFP⁺ cells are colored by green. (G) Dclk1 staining (red) of R26-NGF mice, Tff2-Cre; R26-NGF (green) mice, Tff2-Cre; R26-NGF mice treated with PLX for 1 month, and Tff2-Cre; R26-NGF mice treated with PLX for 1 month and subsequently treated with normal diet for another 1 month. (H-J) Co-culture experiment of sorted Dclk1+ stromal cells (red) and Tff2-Cre; R26-NGF gastric organoids (green). (H) Fluorescence-activated cell sorting plot of Dclk1-CreERT; R26-TdTomato mouse stomach with EpCAM staining 1 day after tamoxifen induction. Cells in the left rectangular outline represent Dclk1+ neurons, and cells in the right outline represent Dclk1⁺ tuft cells. (I) Day 1 and 5 neurite growth image in NGF⁺ organoid (green) co-culture. Arrows indicate Dclk1⁺ neurons (red). (J) Quantification of length of neurite growth. The length with WT organoid co-culture is set as 1.0. n=20/group. Means±SEM. *p<0.05 (t test). DAPI, blue. Scale bars, 100 mm. See also FIGS. 48A-R.

FIGS. 42A-F. ACh/M3R Signaling Regulates Mucosal Proliferation and Stem Cell Expansion. (A) Cross-sectional images of Lgr5-CreERT2; R26-Confetti mice (Chrm3WT/WT) and Lgr5-CreERT; Chrm3^{flox/flox}; R26-Confetti mice. Mice were treated with tamoxifen, following with or without five cycles of MNU. DAPI, white. (B) Percentages of the glands that were fully traced by single color per total glands where recombination occurs. A total of 80 glands from four mice per group was analyzed. (C and D) The number of Ki67⁺ cells per gland (C) and Ki67 staining (D) in WT and Dclk1-CreERT; R26-DTA mice. Mice were treated with tamoxifen on day 1, and given bethanechol for 5 days. A total of 90 glands from three mice per group was analyzed. (E and F) Ki67 staining (E) and the number of Ki67+ cells per gland (F) in R26-NGF, Tff2-Cre; Chrm3^{flox/flox}, Tff2-Cre; R26-NGF, and Tff2-Cre; R26-NGF; Chrm3^{flox/flox} mice. A total of 90 glands from three mice per group was analyzed. Means±SEM. *p<0.05 (ANOVA). Scale bars, 100 mm. See also FIGS. 49A-I.

FIGS. 43A-I. Initiating the ACh-NGF Axis Is Sufficient to Cause Gastric Cancer. (A) H&E (left), Ki67 (middle), and CD44 (right) staining of 8-month-old Tff2-Cre; R26-NGF mice. (B) Gross picture and H&E staining of an 18-month-old Tff2-Cre; R26-NGF mouse stomach. Arrow indicates tumor. (C-E) MNU-induced tumors in R26-NGF (n=19), Tff2-Cre; R26-NGF (n=16), and Tff2-Cre; R26-NGF; Chrm3^{flox/flox} (n=6) mice. Mice were euthanized at 48 weeks after the beginning of MNU. Gross picture (C), H&E image (D), and tumor area (cm²) (E) are shown. Arrows indicate tumors. (F and G) MNU-treated Dclk1-CreERT; R26-DTR mice were treated with vehicle (n=7) or DT (10 mg/kg, n=13). Vehicle or DT and tamoxifen were given once a week from 28 to 52 weeks after the beginning of MNU, then mice were euthanized. Representative tumor image (F) and tumor area (G) are shown. (H and I) Gross images (H) and tumor area (I) of MNU-treated R26-NGF and Tff2-Cre; R26-NGF mice with or without PLX treatment. PLX was given from 24 to 36 weeks after the beginning of MNU, then mice were sacrificed. n=4/group. Average tumor area is indicated by black bars. Means±SEM. *p<0.05; ANOVA in (E), t test in (G) and (I). Scale bars, 100 mm in (A), (B) (right), and (D), 5 mm in (B) (left), (C), (F), and (H). See also FIGS. 50A-Q.

FIGS. 44A-H. M3R Signaling Regulates Apc-Dependent Tumor Growth through YAP Activation. (A-C) Gross pictures (A) and H&E images (B) of Mist1-CreERT; Mist1-CreERT; Apc^{flox/flox·}, Chrm3^{flox′WT}, and Mist1-CreERT; Apc^{flox,flox}, Mist1-CreERT; Apc^{flox/flox·}, Chrm3^{flox′WT}, and Mist1-CreERT; Apc^{flox/flox·}, Chrm3^{flox/flox} mouse tumors. Lines indicate tumor area. Tumor area (cm²) is quantified in (C). Average tumor area is indicated by black bars. (D-G) Ngf gene expression per Gapdh (D) (n=3) and immunofluorescence of NGF (E) (red) and double staining (F) of YAP (red) and b-catenin (green) in Mist1-CreERT; Apc^{flox/flox} and Mist1-CreERT; Apc^{flox/flox}; Chrm3^{flox/flox} mice. Right panels in (F) are enlarged images of white box area in left panels. Numbers of YAP cells in 100 nuclear b-catenin⁺ cells are shown in (G). A total of 500 nuclear b-catenin⁺ cells per group was analyzed. (H) Fold changes of YAP-related gene expression in mouse gastric tumor on the vagotomized side compared with tumor on the non-vagotomized side. Means±SEM. *p<0.05; ANOVA in (C), t test in (D) and (G). DAPI, blue. Scale bars, 100 mm (B), (E), and (F), 5 mm (A). See also FIGS. 51A-J.

FIGS. 45A-I M3R Activates YAP Signaling in Human Gastric Cancer Cells. (A) Immunoblotting of TMK-1 cells treated with 1 mM carbachol for the indicated times. Cells were pretreated with vehicle or 10 mM YM254890. b-Actin was used as a loading control. (B-D) Relative YAP luciferase activity (B) (n=3/group), immunoblotting (C), and relative gene expression (D) (n=3/group) in AGS cells transfected with the indicated amount of control or M3R-expressing vectors. Samples are collected 24 hr after transfection. (E) Representative images of YAP, NGF, and ChAT staining in human gastric cancers. (F and G) Correlation between the expression levels of YAP and NGF (F), and of YAP and ChAT (G) in 36 gastric cancer cases. (H and I) NGF positivity in different cancer stages (H) and histological forms (I) in 97 gastric cancer cases. Means±SEM. *p<0.05; t test in (B) and (D), Fisher's test in (F) and (G). Scale bars, 200 mm. See also FIGS. 52A-G, FIG. 53, and FIG. 54.

FIGS. 46A-F. ChAT and Dclk1 Expression in the Gut. (A) Dclk1 staining (red) on Chat-GFP mouse intestine and colon. (B) Peripherin staining (red) on Chat-GFP mice (left) and Lgr5-GFP expression (right) in the intestine and colon. (C) S100B staining (red) on Chat-GFP mice. (D) Dclk1 staining (green) on Dclk1-CreERT; R26-TdTomato (red) mouse stomach 1 day after tamoxifen. (E) ACh staining (green) on Dclk1-CreERT; R26-TdTomato (red) mouse stomach 1 day after tamoxifen. (F) αSMA, CD31, CD45, and NG2 staining (green) on Dclk1-CreERT; R26-TdTomato (red) mouse 1 day after tamoxifen. Nuclei are counterstained with DAPI (blue). Bars=100 μm (A, C-E), 20 μm (B, F).

FIGS. 47A-D. Cholinergic Signal Regulates NGF Expression in Gastric Epithelium. (A) Relative gene expression per Gapdh in cultured gastric organoids treated with different doses of carbachol (0, 0.1 mM, and 1 mM) for 7 days. Organoids were isolated from WT and M3R knockout (M3R KO) mice. (B) Relative gene expression per Gapdh AOM-DSS induced colon tumor and non-tumor tissues. (C) NGF staining in MNU tumor. (D) Relative Ngf expression per Gapdh in cultured organoids which isolated from MNU tumor. RNA was extracted at day 1 and day 7, and treated with or without carbachol for 7 days. n=3/group. Bars=100 μm. Means±SEM. *p<0.05 (ANOVA).

FIGS. 48A-R. NGF Induces Innervation in the Gastrointestine. (A) Gene construct of Tff2-BAC-Cre transgenic mice. (B) The stomach (corpus and antrum) of Tff2-Cre; R26-mTmG mice. (C) Relative Ngf expression per Gapdh in R26-NGF mice and Tff2-Cre; R26-NGF mice. n=3/group. (D) (left) Dclk1 (gray) and VAChT (red) staining in Tff2-Cre; R26-NGF (green) mice. (right) Dclk1 (red) and TH (green) staining in Tff2-Cre; R26-NGF (green) mice. (E-F) HuC/D staining (E, red) and the numbers of HuC/D+ cells per ganglia (F, n=10) in R26-NGF mice and Tff2-Cre; R26-NGF (green) mice. (G) S 100B staining (red) in Nes-GFP mice and Tff2-Cre; R26-NGF (green); R26-TdTomato (red); Nes-GFP (green) mice. (H) PGP9.5, HuC/D, and Dclk1 staining (red) in Nes-GFP mice and Tff2-Cre; R26-NGF; Nes-GFP (green) mice. (I) αSMA and CD31 staining (red) in Nes-GFP (green) mice. (J-K) H&E (J) and peripherin (red, K) staining in R26-NGF and Vil1-Cre; R26-NGF (green) mouse intestine and colon. (L-M) Dclk1 (green) and BrdU (red) staining of R26-NGF mice, Tff2-Cre; R26-NGF mice, Tff2-Cre; R26-NGF mice treated with PLX for 1 month, and Tff2-Cre; R26-NGF mice treated with PLX for 1 month and subsequently treated with normal diet for another 1 month (L). BrdU in drinking water was administered for 2 months before sacrifice. The numbers of Dclk1-cells per ganglia (n=10) in each group were shown in (M). (N-O) Dclk1-CreERT; R26-mTmG tracing (N) and cell position (O) in the stomach at day 3 and day 360 after tamoxifen induction. Total 50 glands per group are analyzed. (P-Q) Dclk1-CreERT; R26-mTmG tracing (P) and cell position (Q) at day 90 after tamoxifen with control or PLX-7486 treatment. Total 50 glands per group are analyzed. (R) Dclk1-CreERT; R26-mTmG tracing in MNU tumor. Tamoxifen was given at the end of MNU treatment and mice were analyzed 6 months later. Bars=100 μm (B, D, J, K, N, P, R), 20 μm (E, G-I, L). Means±SEM. *p<0.05 (t-test in C and F, ANOVA in M). Nuclei are counterstained with DAPI (blue). The R26-mTmG reporter mouse features the dichotomous expression of red (without Cre-dependent recombination) or green fluorescence (with recombination).

FIGS. 49A-I M3R Signal Regulates Mucosal Proliferation and Regeneration. (A-B) Ki67 staining of WT and Tff2-Cre; Chrm3^{flox/flox} mice treated with 5 cycles of MNU (A) and quantification (B) of Ki67⁺ cells per gland in WT and Tff2-Cre; Chrm3^{flox/flox} mice treated with or without 5 cycles of MNU. n=3/group. Total 60 glands per group are analyzed. (C-D) Day 7 H&E staining (C) of DSS-treated colon from WT and Dclk1-CreERT; R26-DTA mice. Mice were treated with or without bethanechol for 7 days. Tamoxifen was given at day 3. Quantification of ulcer length (D) is shown. n=3/group. (E-F) Day 7 H&E staining (E) of DSS-treated colon from R26-NGF, Vil1-Cre; R26-NGF, Vil1-Cre; Chrm3^{flox/flox}, and Vil1-Cre; R26-NGF; Chrm3^{flox/flox} mice. Quantification of ulcer length (F) is shown. n=3 /group. (G-H) Ki67 staining (G) and quantification (H) in WT, Tff2-Cre; R26-NGF, and Tff2-Cre; R26-NGF; Adrb2^{flox/flox} mice. n=3/group. Total 60 glands per group are analyzed. (I) Ki67 (brown) and peripherin (red) staining of Tff2-Cre; R26-NGF mice, Tff2-Cre; R26-NGF mice treated with PLX-7486 for 1 month, and Tff2-Cre; R26-NGF mice treated with PLX-7486 for 1 month and subsequently with normal diet for another 1 month. NGF Tff2-Cre lineage cells are colored by green. Bars=100 μm. Means±SEM. *p<0.05 (ANOVA). ns; not significant. DAPI=blue.

FIGS. 50A-Q. NGF Promotes Cancer Development in the Gut. (A) Incidence of metaplasia and dysplasia in R26-NGF, Tff2-Cre; R26-NGF, Tff2-Cre; R26-NGF; Chrm3^{flox/flox} mice at 8 months old. (B) H&E and Ki67 staining in 6 months old Vil1-Cre; R26-NGF mouse colon. (C) H&E and Ki67 staining of 8 months old Tff2-Cre; R26-NGF mice and Tff2-Cre; R26-NGF; Chrm3^{flox/flox} mice. (D-F) Tumor image (D) and number (E) and size (F) of AOM-DSS-treated R26-NGF (n=12) and Vil1-Cre; R26-NGF (n=11) mice. Bars in (E) and (F) indicate mean numbers in each group. (G-H)Tumor image (G) and area (H) of MNU-treated stomach of control (n=7) and Adrb2 knockout (Adrb2 KO, n=8) mice. Bars in (H) indicate mean numbers in each group. (I-K) NGF staining (I, red) and peripherin staining (J, red) in MNU tumors from Dclk1-CreERT; R26-DTR mice treated with vehicle or DT as shown in FIG. 5. The relative threshold intensity of positive cells per field is quantified in K (n=3). The mean intensity in control group is set as 1.0. (L) GAP43 (red) and CD44 (green) staining in MNU tumor treated with or without PLX. (M) β-catenin staining in MNU tumor treated with or without PLX. (N) Dclk1 staining (red) in MNU-treated R26-NGF and Tff2-Cre; R26-NGF (green) mice with control or PLX-7486 administration. (O-Q) Allograft model. Lgr5-CreERT; Apc^{flox/flox}; LSL-Tp53^{R172H/+}; LSL-Kras^{G}12^{D/+} mouse organoids were cultured. Day 7 organoids were implanted into NOD-SCID mice subcutaneously and followed for 3 weeks. PLX-7486 or control diet were given from day 7 to day 21. Macroscopic tumor image (O) and tumor size (mm) (P) are shown. (Q) H&E and Dclk1 staining in allograft tumors. n=4/group. Bars=100 μm (B, C, I, J, L-N, Q), 5 mm (D, G, O). Means±SEM. *p<0.05 (t-test). Nuclei are counterstained with DAPI (blue) or hematoxylene (purple).

FIGS. 51A-J. YAP Regulation in an Ace-dependent Gastric Tumor Model through M3R. (A) Lineage tracing of Mist1-CreERT; R26-TdTomato (red) mouse antrum 6 weeks after tamoxifen. (B) Gross picture of Mist1-CreERT; Apc^{flox/flox} mouse stomach 6 weeks after tamoxifen. Tumor develops only in the antrum, not in the corpus. (C-D) Peripherin staining (red, C) in Mist1-CreERT; Apc^{flox/flox} and Mist1-CreERT; Apc^{flox/flox}; Chrm3^{flox/flox} mouse tumors. The relative threshold intensity of peripherin or NGF positive cells per field is quantified in D (n=3). The mean intensity in Mist1-CreERT; Apc^{flox/flox} group is set as 1.0. (E) β-catenin staining in Mist1-CreERT; Apc^{flox/flox} and Mist1-CreERT; Apc^{flox/flox}; Chrm3^{flox/flox} mouse stomach 2 and 6 weeks after tamoxifen. (F) YAP staining in WT stomach. (G) Tamoxifen-treated WT and Mist1-CreERT; Apc^{flox/flox} gastric organoids were cultured and treated with or without 1 mM carbachol for 7 days. Protein lysates were immunoblotted with the indicated antibodies. (H-J) Immunofluorescence (red) of BCL2L1 (H), CD44 (I) or Sox9 (J) with β-catenin doublestain (green) in Mist1-CreERT; Apc^{flox/flox} and Mist1-CreERT; Apc^{flox/flox}; Chrm3^{flox/flox} mouse tumors. Means±SEM. *p<0.05 (t-test). Bars=100 μm (A, C, E, F, H-J), 5 mm (B). Nuclei are counterstained with DAPI (blue) or hematoxylene (purple).

FIGS. 52A-G. YAP Activation and NGF Expression in Human Cancers. (A) Immunoblotting of AGS cells transfected with control or M3R-expressing vectors treated with vehicle or 10 μM YM254890. Samples are treated with drugs 24 hours after transfection, and collected 24 hours after treatment. (B) Immunoblot of TMK-1 cells treated with NGF for the indicated times. (C) (top) Relative NGF expression per GAPDH in TMK-1 cells treated with or without 10 mM carbachol for 24 hours (n=3). (bottom) Relative NGF expression per GAPDH in AGS cells transfected with control or M3R vectors (n=3). (D-E) Relative NGF expression per GAPDH in the indicated cancer cell lines (D, n=2) and NGF protein levels per 1 mg total protein in cell lysates of the indicated cell lines (E, n=2). (F) H&E and NGF staining in human gastric adenoma (dysplasia) and cancer. (G) YAP staining in normal human stomach. Means±SEM. *p<0.05 (t-test). Bars=100 μm.

FIG. 53. Association between NGF expression and clinical and histopathological parameters.

FIG. 54. Association between YAP expression and clinical and histopathological parameters.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington: The Science and Practice of Pharmacy, 21^{st} Edition (University of the Sciences in Philadelphia, ed., Lippincott Williams & Wilkins 2005); and Handbook of Pharmaceutical Excipients, 7^{th} Edition (Raymond Rowe et al., ed., Pharmaceutical Press 2012); each hereby incorporated by reference in its entirety.

The terms “animal,” “subject” and “patient” as used herein includes all members of the animal kingdom including, but not limited to, mammals, animals (e.g., cats, dogs, horses, swine, etc.) and humans.

Methods of Treatment

Methods of Treatment Using Surgical and/or Chemical Denervation

Described herein is the role of nerves in the initiation and progression of gastric cancer. Surgical or chemical denervation of gastric tumors strongly inhibited tumor growth, and lead to tumor regression. Both chemical (e.g. Botox) and surgical denervation therapy can synergize strongly with chemotherapy. The muscarinic M3 receptor was upregulated following denervation, suggesting a receptor target for signaling by the vagal nerve.

Described herein is the role of cholinergic signaling in the regulation of stem cells from the stomach. In an in vitro organoid culture model, it was shown that nerves could stimulate growth of gastric stem cells, which could be blocked by Botox or broad cholinergic antagonists, such as, scopolamine. In addition, broad cholinergic agonists, such as, pilocarpine, could stimulate the growth of gastric stem cells. Furthermore, analysis of Lgr5+ stem cells showed the highest levels of M3 receptor expression in Lgr5(high) cells indicating a selective expression pattern in stem/progenitor cells. Pilocarpine also upregulated Lgr5 gene expression, suggesting it selectively expanded Wnt signaling and stem cells.

In some embodiments, the invention is directed to cholinergic blockade, including specific blockade of the M3 receptor, but covering all muscarinic receptors. In some embodiments, cholinergic blockade is used as an adjunct to traditional forms of cytotoxic therapy, including chemotherapy and radiation therapy. In some embodiments, cholinergic blockade is achieved using scopolamine. Other anticholinergics, including all known pharmaceutical drugs and molecules that are known to have anti-cholinergic effects can also be used. In some embodiments, cholinergic blockade is achieved using amitriptyline or other tricyclic antidepressants. In some embodiments, cholinergic blockade is used as a cancer treatment. In some embodiments, cholinergic blockade is used as a cancer treatment in combination with chemotherapy.

Drugs such as scopolamine, amitriptyline, and other tricyclic antidepressants have an established safety profile and are widely used for a number of clinical indications. These drugs are known to block acetylcholine receptors (including the muscarinic receptors). Anticholinergic drugs can be given at moderate doses safely to patients (e.g. scopolamine patches have been used for motion sickness). In addition, many existing drugs have anticholinergic effects (e.g. tricyclic antidepressants) and ones with the most anticholinergic effects (e.g. amitriptyline) could be used in combination with chemotherapy and may increase the efficacy of treatment. Gastric stem cells have also been shown to respond to stimulation by the neurotransmitter acetylcholine.

Described herein is the removal of cholinergic input to tumors to increase tumor killing, in gastric cancer and other cancers (e.g. colon cancer, esophageal adenocarcinoma). In other embodiments, gastric cancers can be treated by vagotomy, Botox administration, administering an ACh inhibitor, administering a NGF inhibitor, administering a TRK inhibitor, or by targeting the microenvironment of tumors.

Described herein is a new adjuvant treatment to compliment standard treatment courses. Eliminating the innervation of gastric tumor cells, specifically that which relies on muscarinic acetylcholine receptors, plays an important role in the development of gastric stem cells and gastric tumor cells. When this acetylcholine signal was removed or blocked, gastric tumor cells demonstrated slowed growth and even regression. Described herein is the use of acetylcholine blocking drugs, which are already marketed in a variety of forms for many different conditions, as an adjuvant to chemotherapy treatment for gastric cancer. As well as being a new therapeutic target for cancer treatment, this technology provides a new indication for existing drugs such as amitriptyline and tricyclic antidepressants with known anticholinergic activity.

Described herein is an in vitro organoid culture models showing that gastric cancer stem cells grow in response to nerve signaling, specifically the M3 muscarinic receptor. These studies demonstrated blocking such signaling (via Botox or other muscarinic blockade) results in slowing or regression of cell growth. Stem cell studies suggest the mechanism of the effect involves Lgr5 gene expression and the Wnt signaling pathway. Imposing a cholinergic blockade using drugs such as scopalomine, amitriptyline, and many others may have a synergistic effect with chemotherapy and other cancer treatments.

Further information on gastric cancers and the treatment of cancers can be sound in Siegel R, Naishadham D, Jemal A. Cancer statistics, 2013. CA Cancer J Clin 2013; 63:11; Wang W, et al. NNK enhances cell migration through α7-nicotinic acetylcholine receptor accompanied by increased of fibronectin expression in gastric cancer. Ann Surg Oncol. 2012 Jul.;19 Suppl 3:S580-8; Gotoda T. Endoscopic resection of early gastric cancer: the Japanese perspective. Curr Opin Gastroenterol 2006; 22:561; Ronellenfitsch U, Schwarzbach M, Hofheinz R, et al. Perioperative chemo(radio)therapy versus primary surgery for resectable adenocarcinoma of the stomach, gastroesophageal junction, and lower esophagus. Cochrane Database Syst Rev 2013; WO/1994/005271; US Patent Application 20090232849; WO/2012/071573; US Patent Application 20090010923; Spindel ER, Handb Exp Pharmacol., Vol. 208, pp. 451-68, each of which is herein incorporated by reference in their entireties.

In certain aspects, the invention provides a method for treating cancer in a subject in need thereof, the method comprising administering to the subject a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for treating cancer in a subject in need thereof, the method comprising performing surgical denervation.

In some embodiments, the invention provides a method of treating cancer in a subject in need thereof, the method comprising administering to the subject a cholinergic antagonist. In some embodiments, the invention provides a method for treating cancer in a subject in need thereof, the method comprising administering to the subject Botulinum toxin. In some embodiments, the invention provides a method for treating cancer in a subject in need thereof, the method comprising administering to the subject an ACh inhibitor. In some embodiments, the invention provides a method for treating cancer in a subject in need thereof, the method comprising administering to the subject a NGF inhibitor. In some embodiments, the invention provides a method for treating cancer in a subject in need thereof, the method comprising administering to the subject a TRK inhibitor, In some embodiments, the invention provides a method for treating cancer in a subject in need thereof, the method comprising performing surgical denervation. In some embodiments, the invention provides a method of treating cancer in a subject in need thereof, the method comprising administering a cholinergic antagonist, Botulinum toxin, an ACh inhibitor, a NGF inhibitor, a TRK inhibitor, performing surgical denervation, or a combination thereof.

In some embodiments, the invention provides a method of treating cancer in a subject in need thereof, the method comprising administering a cholinergic antagonist, Botulinum toxin, an ACh inhibitor, a NGF inhibitor, a TRK inhibitor,or performing surgical denervation in combination with a cytotoxic therapy. In some embodiments the cholinergic antagonist, Botulinum toxin, ACh inhibitor, NGF inhibitor, or TRK inhibitor, is administered before, during, or after the administration of the cytotoxic therapy. In some embodiments, the cytotoxic therapy is radiotherapy or chemotherapy.

In some embodiments, the invention provides a method of treating cancer in a subject in need thereof, the method comprising administering a cholinergic antagonist, Botulinum toxin, an ACh inhibitor, a NGF inhibitor, a TRK inhibitor, or performing surgical denervation in combination with surgery. In some embodiments the cholinergic antagonist, Botulinum toxin, ACh inhibitor, NGF inhibitor, or TRK inhibitor, is administered before, during, or after the surgery. In some embodiments, the surgery is endoscopic resection or gastrectomy.

In some embodiments, the cancer is gastric cancer. In some embodiments, the cancer is colon cancer. In some embodiments, the subject is at high risk of developing gastric cancer. In some embodiments, the subject is at high risk of developing colon cancer.

In some embodiments the cholinergic antagonist is a small molecule. In some embodiments, the cholinergic antagonist is scopolamine. In some embodiments, the cholinergic antagonist is amitriptyline. In some embodiments, the cholinergic antagonist is a tricyclic antidepressant. In some embodiments, the cholinergic antagonist blocks the M3 muscarinic receptor.

In some embodiments the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486. In some embodiments, the TRK inhibitor is an anti-TRK antibody. Other TRK inhibitors are described in Table 2 of Vaishnavi, A., Le, A. T., and Doebele, R. C. (2015), TRKing down an old onco-gene in a new era of targeted therapy, Cancer Discov. 5, 25-34, the contents of which are hereby incorporated by reference.

In certain aspects, the invention provides a method for reducing proliferation of tumor cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for reducing proliferation of tumor cells, the method comprising performing surgical denervation.

In certain aspects, the invention provides a method for inhibiting proliferation of tumor cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for inhibiting proliferation of tumor cells, the method comprising performing surgical denervation.

In certain aspects, the invention provides a method for inhibiting tumor metastasis, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for inhibiting tumor metastasis, the method comprising performing surgical denervation.

In some embodiments, the invention provides a method for reducing proliferation of tumor cells, a method for inhibiting proliferation of tumor cells, or a method for inhibiting tumor metastasis, the method comprising administering a cholinergic antagonist. In some embodiments, the invention provides a method for reducing proliferation of tumor cells, a method for inhibiting proliferation of tumor cells, or a method for inhibiting tumor metastasis, the method comprising administering Botulinum toxin. In some embodiments, the invention provides a method for reducing proliferation of tumor cells, a method for inhibiting proliferation of tumor cells, or a method for inhibiting tumor metastasis, the method comprising administering an ACh inhibitor. In some embodiments, the invention provides a method for reducing proliferation of tumor cells, a method for inhibiting proliferation of tumor cells, or a method for inhibiting tumor metastasis, the method comprising administering a NGF inhibitor. In some embodiments, the invention provides a method for reducing proliferation of tumor cells, a method for inhibiting proliferation of tumor cells, or a method for inhibiting tumor metastasis, the method comprising administering a TRK inhibito. In some embodiments, the invention provides a method for reducing proliferation of tumor cells, a method for inhibiting proliferation of tumor cells, or a method for inhibiting tumor metastasis, the method comprising performing surgical denervation. In some embodiments, the invention provides a method for reducing proliferation of tumor cells, a method for inhibiting proliferation of tumor cells, or a method for inhibiting tumor metastasis, the method comprising administering a cholinergic antagonist, Botulinum toxin, an ACh inhibitor, a NGF inhibitor, a TRK inhibitor, performing surgical denervation, or a combination thereof.

In some embodiments, the invention provides a method for reducing proliferation of tumor cells, a method for inhibiting proliferation of tumor cells, or a method for inhibiting tumor metastasis, the method comprising administering a cholinergic antagonist, Botulinum toxin, an ACh inhibitor, a NGF inhibitor, a TRK inhibitor, or performing surgical denervation in combination with a cytotoxic therapy. In some embodiments the cholinergic antagonist, Botulinum toxin, ACh inhibitor, NGF inhibitor, or TRK inhibitor, is administered before, during, or after the administration of the cytotoxic therapy. In some embodiments, the cytotoxic therapy is radiotherapy or chemotherapy.

In some embodiments, the invention provides a method for reducing proliferation of tumor cells, a method for inhibiting proliferation of tumor cells, or a method for inhibiting tumor metastasis, the method comprising administering a cholinergic antagonist, Botulinum toxin, an ACh inhibitor, a NGF inhibitor, a TRK inhibitor, or performing surgical denervation in combination with surgery. In some embodiments the cholinergic antagonist, Botulinum toxin, ACh inhibitor, NGF inhibitor, or TRK inhibitor is administered before, during, or after the surgery. In some embodiments, the surgery is endoscopic resection or gastrectomy.

In some embodiments, the invention provides a method for reducing proliferation of tumor cells, a method for inhibiting proliferation of tumor cells, or a method for inhibiting tumor metastasis in a subject in need thereof. In some embodiments, the subject has gastric cancer. In some embodiments, the subject has colon cancer. In some embodiments, the subject is at high risk of developing gastric cancer. In some embodiments, the subject is at high risk of developing colon cancer. In some embodiments, the tumor cells are gastric tumor cells. In some embodiments, the tumor cells are colon tumor cells. In some embodiments, the tumor is a gastric tumor. In some embodiments, the tumor is a colon tumor.

In some embodiments the cholinergic antagonist is a small molecule. In some embodiments, the cholinergic antagonist is scopolamine. In some embodiments, the cholinergic antagonist is amitriptyline. In some embodiments, the cholinergic antagonist is a tricyclic antidepressant. In some embodiments, the cholinergic antagonist blocks the M3 muscarinic receptor.

In some embodiments the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486. In some embodiments, the TRK inhibitor is an anti-TRK antibody.

In certain aspects, the invention provides a method for treating tumor reoccurrence in a subject in need thereof, the method comprising administering to the subject a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for treating tumor reoccurrence in a subject in need thereof, the method comprising performing surgical denervation.

In some embodiments, the invention provides a method for treating tumor reoccurrence in a subject in need thereof, the method comprising administering to the subject a cholinergic antagonist. In some embodiments, the invention provides a method for treating tumor reoccurrence in a subject in need thereof, the method comprising administering to the subject Botulinum toxin. In some embodiments, the invention provides a method for treating tumor reoccurrence in a subject in need thereof, the method comprising administering to the subject an ACh inhibitor. In some embodiments, the invention provides a method for treating tumor reoccurrence in a subject in need thereof, the method comprising administering to the subject a NGF inhibitor. In some embodiments, the invention provides a method for treating tumor reoccurrence in a subject in need thereof, the method comprising administering to the subject a TRK inhibitor, In some embodiments, the invention provides a method for treating tumor reoccurrence in a subject in need thereof, the method comprising performing surgical denervation. In some embodiments, the invention provides a method for treating tumor reoccurrence in a subject in need thereof, the method comprising administering a cholinergic antagonist, Botulinum toxin, an ACh inhibitor, a NGF inhibitor, a TRK inhibitor, performing surgical denervation, or a combination thereof.

In some embodiments, the invention provides a method for treating tumor reoccurrence in a subject in need thereof, the method comprising administering a cholinergic antagonist, Botulinum toxin, an ACh inhibitor, a NGF inhibitor, a TRK inhibitor, or performing surgical denervation in combination with a cytotoxic therapy. In some embodiments the cholinergic antagonist, Botulinum toxin, ACh inhibitor, NGF inhibitor, or TRK inhibitor, is administered before, during, or after the administration of the cytotoxic therapy. In some embodiments, the cytotoxic therapy is radiotherapy or chemotherapy.

In some embodiments, the invention provides a method for treating tumor reoccurrence in a subject in need thereof, the method comprising administering a cholinergic antagonist, Botulinum toxin, an ACh inhibitor, a NGF inhibitor, a TRK inhibitor, or performing surgical denervation in combination with surgery. In some embodiments the cholinergic antagonist, Botulinum toxin, ACh inhibitor, NGF inhibitor, or TRK inhibitor, is administered before, during, or after the surgery. In some embodiments, the surgery is endoscopic resection or gastrectomy.

In some embodiments, the subject has gastric cancer. In some embodiments, the subject has pancreatic cancer. In some embodiments, the subject has colon cancer. In some embodiments, the subject is at high risk of developing gastric cancer. In some embodiments, the subject is at high risk of developing colon cancer. In some embodiments, the tumor is a gastric tumor. In some embodiments, the tumor is a colon tumor.

In some embodiments the cholinergic antagonist is a small molecule. In some embodiments, the cholinergic antagonist is scopolamine. In some embodiments, the cholinergic antagonist is amitriptyline. In some embodiments, the cholinergic antagonist is a tricyclic antidepressant. In some embodiments, the cholinergic antagonist blocks the M3 muscarinic receptor.

In some embodiments the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486. In some embodiments, the TRK inhibitor is an anti-TRK antibody.

In certain aspects, the invention provides a method for inhibiting stem cell growth, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for inhibiting stem cell growth, the method comprising performing surgical denervation.

In certain aspects, the invention provides a method for inhibiting Wnt signaling in stem cells, the method comprising administering a cholinergic antagonist, a Botulinum toxin, an ACh inhibitor, a NGF inhibitor, a TRK inhibitor, or a combination thereof. In some embodiments, the method further comprises performing surgical denervation.

In certain aspects, the invention provides a method for inhibiting Wnt signaling in stem cells, the method comprising performing surgical denervation.

In some embodiments, the invention provides a method for inhibiting stem cell growth, or a method for inhibiting Wnt signaling in stem cells, the method comprising administering a cholinergic antagonist. In some embodiments, the invention provides a method for inhibiting stem cell growth, or a method for inhibiting Wnt signaling in stem cells, the method comprising administering Botulinum toxin. In some embodiments, the invention provides a method for inhibiting stem cell growth, or a method for inhibiting Wnt signaling in stem cells, the method comprising administering an ACh inhibitor. In some embodiments, the invention provides a method for inhibiting stem cell growth, or a method for inhibiting Wnt signaling in stem cells, the method comprising administering a NGF inhibitor. In some embodiments, the invention provides a method for inhibiting stem cell growth, or a method for inhibiting Wnt signaling in stem cells, the method comprising administering a TRK inhibitor. In some embodiments, the invention provides a method for inhibiting stem cell growth, or a method for inhibiting Wnt signaling in stem cells, the method comprising performing surgical denervation. In some embodiments, the invention provides a method for inhibiting stem cell growth, or a method for inhibiting Wnt signaling in stem cells, the method comprising administering a cholinergic antagonist, Botulinum toxin, an ACh inhibitor, a NGF inhibitor, a TRK inhibitor, performing surgical denervation, or a combination thereof.

In some embodiments, the invention provides a method for inhibiting stem cell growth, or a method for inhibiting Wnt signaling in stem cells, the method comprising administering a cholinergic antagonist, Botulinum toxin, an ACh inhibitor, a NGF inhibitor, a TRK inhibitor, or performing surgical denervation in combination with a cytotoxic therapy. In some embodiments the cholinergic antagonist, Botulinum toxin, ACh inhibitor, NGF inhibitor, or TRK inhibitor, is administered before, during, or after the administration of the cytotoxic therapy. In some embodiments, the cytotoxic therapy is radiotherapy or chemotherapy.

In some embodiments, the invention provides a method for inhibiting stem cell growth, or a method for inhibiting Wnt signaling in stem cells, the method comprising administering a cholinergic antagonist, Botulinum toxin, an ACh inhibitor, a NGF inhibitor, a TRK inhibitor, or performing surgical denervation in combination with surgery. In some embodiments the cholinergic antagonist, Botulinum toxin, ACh inhibitor, NGF inhibitor, or TRK inhibitor, is administered before, during, or after the surgery. In some embodiments, the surgery is endoscopic resection or gastrectomy.

In some embodiments, the stem cells are gastric cancer stem cells. In some embodiments, the stem cells are colon cancer stem cells. In some embodiments, the stem cells are in a tumor. In some embodiments, the tumor is a gastric tumor. In some embodiments, the tumor is a colon tumor.

In some embodiments, the invention provides a method for inhibiting stem cell growth, or a method for inhibiting Wnt signaling in stem cells in a subject in need thereof. In some embodiments, the subject has gastric cancer. In some embodiments, the subject has colon cancer. In some embodiments, the subject is at high risk of developing gastric cancer. In some embodiments, the subject is at high risk of developing colon cancer

In some embodiments the cholinergic antagonist is a small molecule. In some embodiments, the cholinergic antagonist is scopolamine. In some embodiments, the cholinergic antagonist is amitriptyline. In some embodiments, the cholinergic antagonist is a tricyclic antidepressant. In some embodiments, the cholinergic antagonist blocks the M3 muscarinic receptor.

In some embodiments the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486. In some embodiments, the TRK inhibitor is an anti-TRK antibody.

In some embodiments, the surgical denervation is a vagotomy. In some embodiments, the surgical denervation is a bilateral vagotomy with pyloroplasty. In some embodiments, the surgical denervation is a unilateral vagotomy. In some embodiments, the Botulinum toxin inhibits local signaling from the vagus nerve. In some embodiments the cholinergic antagonist is a small molecule. In some embodiments, the cholinergic antagonist is a muscarinic receptor antagonist. In some embodiments, the cholinergic antagonist is a M3 receptor antagonist. In some embodiments, the cholinergic antagonist is darifenacin, scopolamine, amitriptyline, or a tricyclic antidepressant.

In some embodiments the ACh inhibitor is a small molecule. In some embodiments, the NGF inhibitor is a small molecule. In some embodiments the NGF inhibitor is an anti-NGF antibody, for example, but not limited to fulranumab, tanezumab, or fasinumab. In some embodiments, the TRK inhibitor is a small molecule. In some embodiments, the TRK inhibitor is PLX7486. In some embodiments, the TRK inhibitor is an anti-TRK antibody.

In some embodiments, the method further comprises administering a cytotoxic therapy. In some embodiments, the cholinergic antagonist, Botulinum toxin, ACh inhibitor, NGF inhibitor, TRK inhibitor, or surgical denervation is administered or performed before, during, or after the administration of the cytotoxic therapy. In some embodiments, the cytotoxic therapy is radiotherapy or chemotherapy.

In some embodiments, the method further comprises performing an endoscopic resection surgery or a gastrectomy surgery. In some embodiments, the cholinergic antagonist, Botulinum toxin, ACh inhibitor, NGF inhibitor, TRK inhibitor, or surgical denervation is administered or performed before, during, or after the endoscopic resection surgery or gastrectomy surgery is performed.

In some embodiments, the cancer is gastric cancer. In some embodiments, the cancer is colon cancer. In some embodiments, the tumor is a gastric tumor. In some embodiments, the tumor is a colon tumor. In some embodiments, the stem cells are cancer stem cells. In some embodiments, the cancer stem cells are gastric cancer stem cells. In some embodiments, the cancer stem cells are colon cancer stem cells. In some embodiments, the stem cells express Lgr5. In some embodiments, the stem cells express M3 receptor. In some embodiments, the stem cells are gastric stem cells.

Methods of Treatment Using Cholinergic Agonists

Proctitis, colitis, and other inflammatory bowel diseases, characterized by the inflammation of the tissue lining of the gastrointestinal (GI) tract, affects millions of people nationwide. These conditions are often painful and are a common side effect of radiation therapy and/or chemotherapy for the treatment of cancer.

Radiation therapy and chemotherapy for cancer often induces damage to the gastrointestinal tract, such as proctitis or colitis (Hongjie Li, Heinrich Jasper. Gastrointestinal stem cells in health and disease: from flies to humans. Disease Models & Mechanisms, 2016; 9: 487-499; Brittan M, Wright N A. Stem Cell In Gastrointestinal Structure And Neoplastic Development. Gut, 2004 June; 53(6): 899-910; Amout Schepers, Hans Clevers. Wnt Signaling, Stem Cells, and Cancer of the Gastrointestinal Tract. Cold Spring Harb. Perspect. Biol, 2012; 4: a007989). The gastrointestinal tract is innervated by cholinergic nerves, which directly regulate stem cell production there (Britten et al; Westphalen CB et al. Long-lived intestinal tuft cells serve as colon cancer-initiating cells. J Clin Invest. 2014 Feb.; 124(3): 1283-1295). Described herein are results showing that reduction in the amount of acetylcholine in the intestines in mouse models showed impaired intestinal regeneration, whereas replacement of cholinergic agents improved intestinal regeneration.

Described herein is the use of bethanechol or other cholinergic agonists to improve ability of patients to heal from gastrointestinal damage. Activation of M3R in the gastrointestinal tract stimulates stem cell production, which acts to repair gastrointestinal lining damage and inflammation. Treatments can be administered orally to promote intestinal regeneration or rectally, depending on specific condition and its localization in the gastrointestinal tract. Treatments can be administered both pre- and post-radiation treatment. Significant improvement in the colon health in mice subjected to the radiation treatment is shown.

Described herein is the role of cholinergic signaling in the regulation of stem cells from the stomach. Broad cholinergic agonists, such as, pilocarpine, could stimulate the growth of gastric stem cells. Furthermore, analysis of Lgr5+ stem cells showed the highest levels of M3 receptor expression in Lgr5(high) cells indicating a selective expression pattern in stem/progenitor cells. Pilocarpine also upregulated Lgr5 gene expression, suggesting it selectively expanded Wnt signaling and stem cells.

Described herein is the role of cholinergic signaling in the regulation of regeneration of the colon or stomach. Broad cholinergic agonists, such as, pilocarpine or bethanechol, could stimulate the growth of colon cells, including, colon stem cells. The invention provides methods for protecting and promoting cell growth and restoring physiological structure and function to the digestive system.

In certain aspects, the invention provides a method for stimulating growth of stem cells, the method comprising administering a cholinergic agonist.

In some embodiments, the stem cells are gastric stem cells. In some embodiments, the stem cells are colon stem cells.

In certain aspects, the invention provides a method for stimulating regeneration of the digestive system in a subject in need thereof, the method comprising administering to the subject a cholinergic agonist. In some embodiments, the subject has an inflammatory disease of the digestive system. In one embodiment, the inflammatory disease of the digestive system comprises esophagitis, inflammatory bowel disease, Crohn's disease, ulcerative colitis, colitis, irritable bowel syndrome, celiac disease, gastritis, peptic ulcer disease, pancreatitis or a combination thereof. In another embodiment, the inflammatory disease of the digestive system is colitis. In some embodiments, the subject has an ulcer of the digestive system. In some embodiments, the subject has an acute or chronic digestive system injury, for example, but not limited to radiation-induced proctitis, radiation-induced colitis, chemotherapy-induced proctitis, chemotherapy-induced colitis, ulcerative colitis, radiation induced gastrointestinal syndrome (RIGS), inflammatory bowel disease, or colitis.

In certain aspects, the invention provides a method for stimulating regeneration of the colon in a subject in need thereof, the method comprising administering to the subject a cholinergic agonist.

In certain aspects, the invention provides a method for stimulating regeneration of the stomach in a subject in need thereof, the method comprising administering to the subject a cholinergic agonist.

In certain aspects, the invention provides a method for treating an inflammatory disease of the digestive system in a subject in need thereof, the method comprising administering to the subject a cholinergic agonist. In one embodiment, the inflammatory disease of the digestive system comprises esophagitis, inflammatory bowel disease, Crohn's disease, ulcerative colitis, colitis, irritable bowel syndrome, celiac disease, gastritis, peptic ulcer disease, pancreatitis or a combination thereof. In another embodiment, the inflammatory disease of the digestive system is colitis. In another embodiment, the inflammatory disease of the digestive system is gastritis.

In certain aspects, the invention provides a method for treating radiation-induced proctitis, radiation-induced colitis, chemotherapy-induced proctitis, chemotherapy-induced colitis, or radiation induced gastrointestinal syndrome (RIGS) in a subject in need thereof, the method comprising administering to the subject a cholinergic agonist.

In certain aspects, the invention provides a method for treating a digestive system injury in a subject in need thereof, the method comprising administering to the subject a cholinergic agonist. In one embodiment, the injury is an acute injury. In another embodiment, the injury is a stomach injury. In another embodiment, the injury is a colon injury. In certain aspects, injury is from radiation.

In some embodiments, the cholinergic agonist is a small molecule. In some embodiments, the cholinergic agonist is pilocarpine. In some embodiments, the cholinergic agonist is carbachol. In some embodiments, the cholinergic agonist is aceclidine. In some embodiments, the cholinergic agonist is arecoline. In some embodiments, the cholinergic agonist is cevimeline. In some embodiments, the cholinergic agonist is bethanechol. In some embodiments, the cholinergic agonist is muscarine. In some embodiments, the cholinergic agonist is oxotremorine. In some embodiments, the cholinergic agonist is methacholine. In some embodiments, the cholinergic agonist activates the M3 muscarinic receptor.

Diseases of the Digestive System

The present invention provides methods for treating diseases of the digestive system. In one embodiment the digestive system comprises the gastrointestinal tract including structures from the mouth to the anus, and the accessory organs. For example, this includes, but is not limited to, the mouth, the pharynx, the esophagus, the stomach, the small intestine, including the duodenum, jejunum, and ileum, the large intestine including the cecum, colon, and rectum, and the anus. In further embodiments, the accessory organs of the digestive system include, but are not limited to, the liver, the pancreas, and the gall bladder.

The present invention provides methods for treating a digestive system cancer in a subject, the method comprising administering a cholinergic antagonist, Botulinum toxin, an ACh inhibitor, a NGF inhibitor, a TRK inhibitor, performing surgical denervation, or a combination thereof. In one embodiment, the digestive system cancer includes, but is not limited to, mouth cancer, pharynx cancer, esophageal cancer, gastric cancer, small intestine cancer, large intestine cancer, colon cancer, rectal cancer, anal cancer, liver cancer, and gall bladder cancer. In one embodiment, the digestive system cancer includes, but is not limited to, cancers of gastric origin such as esophageal adenocarcinoma.

In some embodiments, the subject is already suspected to have a digestive system cancer. In other embodiments, the subject is being treated for a digestive system cancer, before being treated according to the methods of the invention. In other embodiments, the subject is not being treated for a digestive system cancer, before being treated according to the methods of the invention.

The present invention also provides methods for decreasing tumor growth in a subject, the method comprising administering a cholinergic antagonist, Botulinum toxin, an ACh inhibitor, a NGF inhibitor, a TRK inhibitor, performing surgical denervation, or a combination thereof. In one embodiment, the tumor is a tumor of the digestive system. In one embodiment, the tumor is a gastric tumor. In one embodiment, the tumor is a colon tumor. Tumor growth can be measured in a variety of ways, known to one of skill in the art. For example, tumor growth can be measured by measuring the tumor volume over time. Tumor volume can be measured in a variety of ways, known to one of skill in the art including, but not limited to, positron emission tomography and computed tomography (PET-CT), single-photon emission computed tomography (SPECT-CT), magnetic resonance spectroscopy (MR), X-ray computed tomography (CT), and molecular imaging.

The present invention provides methods for treating dysplasia of the digestive system in a subject, the method comprising administering a cholinergic antagonist, Botulinum toxin, an ACh inhibitor, a NGF inhibitor, a TRK inhibitor, performing surgical denervation, or a combination thereof. Dysplasia is a condition where there is a morphologically identifiable local tissue abnormality at a given site. Dysplasia can have characteristics including, but not limited to, increased cell number, nuclear abnormalities, and cellular differentiation abnormalities, compared to normal cells. A dysplasia can precede the development of any neoplasm, benign or malignant. In one embodiment, the dysplasia is gastric dysplasia. In one embodiment, the dysplasia is colon dysplasia.

The present invention provides methods for decreasing cell proliferation in a subject, the method comprising administering a cholinergic antagonist, Botulinum toxin, an ACh inhibitor, a NGF inhibitor, a TRK inhibitor, performing surgical denervation, or a combination thereof. In one embodiment, the cells are gastric cancer stem cells. In one embodiment, the cells are colon cancer stem cells. In another embodiment the gastric cancer stem cells are tumor associated. In another embodiment the colon cancer stem cells are tumor associated. In another embodiment, the gastric cancer stem cell is a gastric cancer stem cell associated with a tumor. In another embodiment, the colon cancer stem cell is a colon cancer stem cell associated with a tumor. For example, the tumor can be any solid tumor associated with gastric cancer stem cells or colon cancer stem cells. A tumor is a growth of tissue forming an abnormal mass, and can be benign, pre-malignant, or malignant. In one embodiment, the tumor is a gastric tumor. In another embodiment, the tumor is a pancreatic tumor. In another embodiment, the tumor is a colon tumor.

In a further embodiment gastric cancer stem cells express a marker. In yet another embodiment gastric cancer stem cells do not express a marker. In one embodiment, gastric cancer stem cells express the surface marker Lgr5.

The present invention provides methods for stimulating regeneration of the digestive system in a subject, the method comprising administering a cholinergic agonist. In one embodiment, the regeneration is of the colon. In another embodiment, the regeneration is of the stomach. In some embodiments, the regeneration is of colon cells and/or tissue. In another embodiment, the regeneration is of stomach cells and/or tissue. In another embodiment, the subject has an inflammatory disease of the digestive system. In one embodiment, the inflammatory disease of the digestive system comprises esophagitis, inflammatory bowel disease, Crohn's disease, ulcerative colitis, colitis, irritable bowel syndrome, celiac disease, gastritis, peptic ulcer disease, pancreatitis or a combination thereof. In another embodiment, the inflammatory disease of the digestive system is colitis. In another embodiment, the subject has an ulcer of the digestive system (e.g. a stomach ulcer or a colon ulcer). In another embodiment, the subject has an acute or chronic digestive system injury, for example, but not limited to, radiation-induced proctitis, radiation-induced colitis, chemotherapy-induced proctitis, chemotherapy-induced colitis, ulcerative colitis, radiation induced gastrointestinal syndrome (RIGS), inflammatory bowel disease, or colitis.

The invention provides methods for protecting and promoting cell growth and restoring physiological structure and function to the digestive system. In some embodiments, the present invention can be used to activate or induce regenerative stem cells in tissues in the digestive system so as to repair diseased or damaged tissues of the digestive system by administering a cholinergic agonist. In some embodiments, the present invention can be used to activate adult stem cells in the body to repair damaged tissues and to regenerate dysfunctional digestive system organs in situ and in vivo.

The present invention also provides a kit for treating a gastric cancer in a subject. In one embodiment, the kit for treating a gastric cancer comprises a cholinergic antagonist, a Botulinum toxin, an ACh inhibitor, a NGF inhibitor, a TRK inhibitor, or a combination thereof, to administer to a subject and instructions of use.

The present invention also provides a kit for treating a colon cancer in a subject. In one embodiment, the kit for treating a colon cancer comprises a cholinergic antagonist, a Botulinum toxin, an ACh inhibitor, a NGF inhibitor, a TRK inhibitor, or a combination thereof, to administer to a subject and instructions of use.

The present invention also provides a kit for stimulating growth of stem cells of the digestive system. In one embodiment, the kit for stimulating growth of stem cells of the digestive system comprises a cholinergic agonist to administer to a subject and instructions of use. In some embodiments, the stem cells are gastric stem cells, colon stem cells, or pancreatic stem cells.

The present invention also provides a kit for stimulating regeneration of the digestive system. In one embodiment, the kit for regenerating the digestive system comprises a cholinergic agonist to administer to a subject and instructions of use. In some embodiments, the regeneration is of colon cells and/or tissue. In another embodiment, the regeneration is of stomach cells and/or tissue.

In one embodiment, the subject is an animal. In another embodiment, the subject is an animal that has or is diagnosed with a gastric cancer. In another embodiment, the subject is an animal that has or is diagnosed with a colon cancer. In one embodiment, the subject is a human. In other embodiments, the subject is a mammal. In one embodiment, the subject is a dog. In another embodiment, the subject is a cat. In some embodiments, the subject is a rodent, such as a mouse or a rat. In some embodiments, the subject is a cow, pig, sheep, goat, cat, horse, dog, and/or any other species of animal used as livestock or kept as pets.

Cholinergic Modulators

Cholinergic antagonists can be useful in the methods of the present invention. In some embodiments the cholinergic antagonists can inhibit one or more characteristic responses of a muscarinic receptor. Examples of cholinergic antagonists include, but are not limited to, darifenacin, scopolamine, amitriptyline, or a tricyclic antidepressant.

In some embodiments, the invention relates to methods of treating gastric cancer by inhibiting muscarinic M3 receptors. M3 receptors can be inhibited by a variety of agents, including but not limited to, cholinergic antagonists (e.g. darifenacin, scopolamine, amitriptyline, tricyclic antidepressants), M3 receptor antibodies, or anti-M3 receptor nucleic acid sequences.

Cholinergic agonists can be useful in the methods of the present invention. In some embodiments the cholinergic agonists can activate one or more characteristic responses of a muscarinic receptor. Examples of cholinergic agonists include, but are not limited to pilocarpine, aceclidine, arecoline, cevimeline, bethanechol, muscarine, oxotremorine, carbachol or methacholine.

In some embodiments, the invention relates to methods of stimulating the growth of stem cells by activating muscarinic M3 receptors. M3 receptors can be activated by a variety of agents, including but not limited to, cholinergic agonists (e.g. pilocarpine, aceclidine, arecoline, cevimeline, bethanechol, muscarine, oxotremorine, carbachol, methacholine), M3 receptor agonists, or by overexpression of M3 receptor nucleic acid sequences.

The polynucleotide and polypeptide sequences of the muscarinic M3 receptor are publically available and accessible through sequence databases (e.g. CHRM3 located on chromosome 1q43 (Gene ID 1131); GenBank U29589.1 (DNA); NM_000740 (mRNA); NP_000731.1 (Protein); UniProt P20309 (Protein)). Anti-M3 receptor antibodies and anti-M3 receptor nucleic acid sequences can be designed and made according to methods known to one of skill in the art.

Botulinum Toxin

A Botulinum toxin refers to any one of the naturally occurring botulinum toxins found in Clostridium botulinum species. For example, various different Botulinum toxin types have been isolated and characterized from various strain of C. botulinum. The isolated toxins, included, but are not limited to botulinum toxin type A, botulinum toxin type B, botulinum toxin type C1, botulinum toxin type C2, botulinum toxin type C3, botulinum toxin type D, botulinum toxin type E, botulinum toxin type F and botulinum toxin type G (BTTG).

Type A Botulinum toxin refers to all forms of Botulinum toxin comprising the Clostridium botulinum type A neurotoxin. Botulinum toxin Type A is known to exist naturally in three forms each comprising the type A neurotoxin: the M complex (300 kD) consisting of the neurotoxin polypeptide plus a non-toxic non-hemagglutinin protein of similar size; the L complex (500 kD); the LL complex (900 kD) which consists of a number of proteins with hemagglutinin activity in addition to the proteins in the M complex. These above mentioned forms can fall within the meaning of a Botulinum toxin according to the present invention.

Botulinum toxin may include the commercial products Botox® (Botulinum Toxin Type A Neurotoxin Complex, Allergan), Botox® Cosmetic (Allergan), Vistabel® (Allergan, France), Dysport® (Ipsen Ltd./Beaufour Ipsen), Reloxin™ (Ipsen Ltd./Inamed), Clostridium botulinum type A toxins prepared by Mentor Corporation, Xeomin® (MerZ Pharma, Germany), Linurase® (Prollenium, lnc., Canada), CBTX-A® (Lanzhou Biological Products Institute, China), and Neuronox® (Medy-Tox, Inc., South Korea).

In one embodiment a Botulinum toxin polypeptide is Nc-224 (Allergan and CAMR), a fragment of botulinum toxin standard A lacking the binding domain (LHN/A), conjugated to native Erythrina cryslagalli lectin (ECL).

In one embodiment a Botulinum toxin polypeptide is Nc-270 (Allergan and CAMR), a fragment of botulinum toxin standard A lacking the binding domain (LHN/A), conjugated to recombinant Erylhrina cryslagalli lectin (ECL).

In one embodiment a BTTA polypeptide is a highly purified form of botulinum neurotoxin standard A developed by Ipsen and by Inamed Corporation. It includes the commercial products Dysport® and Reloxin™.

In some embodiments, Botulinum toxin may include a complex-free type-A neurotoxin. Botulinum toxin may also include a polypeptide comprising the sequence of the active BTTA neurotoxin polypeptide. It may also include a polypeptide with a similar identity, homology to, or comprising a functional fragment of the native, active type-A neurotoxin polypeptide.

Nucleic acid encoding Botulinum toxin may comprise a nucleic acid sequence capable of encoding the active type-A neurotoxin polypeptide. It may alternatively comprise a nucleic acid sequence capable of encoding a polypeptide with a similar identity, homology to, or having a functional fragment of the native, active length type-A neurotoxin polypeptide. The polynucleotide and polypeptide sequences of Botulinum toxin are publically available and accessible through sequence databases (e.g. Uniprot P10845; A5HZZ9). Information relating to the sequences of Botulinum toxin can be found in Zhang Let al. (2003) Gene 315:21-32 and Thompson D. E. et al. (1990) Eur. J. Biochem. 189(1):73-81 the contents of which are hereby incorporated by reference in their entireties.

In one embodiment, a Botulinum toxin polypeptide refers to a polypeptide having at least 80% amino acid identity, preferably 85%, 90%, 95%, or higher, up to and including 100% identity, with active Botulinum toxin, and which exhibits a neurotoxic activity e.g. it blocks neurotransmitter release at peripheral cholinergic nerve terminals. A Botulinum toxin polypeptide may also be a functional fragment of active Botulinum toxin and which exhibits a neurotoxic activity e.g. a portion of Botulinum toxin which blocks neurotransmitter release at peripheral cholinergic nerve terminals such as the neuromuscular junction. A functional fragment of a Botulinum toxin polypeptide may comprise at least 10, 20, 30, 40, 50, 60, 70, 80, 90, or 95% of the amino acids of the sequence represented by the native sequence.

A Botulinum toxin polypeptide also refers to an homologous sequence of an active Botulinum toxin polypeptide. Homologous sequences may comprise additions, deletions or substitutions of one or more amino acids or nucleotides, which do not substantially alter the functional characteristics of Botulinum toxin. That is, homologues may have at least 90% of the activity of native, active Botulinum toxin. Homologous sequences of Botulinum toxin can also be nucleotide sequences of more than 50, 100, 200, 300, 400, 600, 800 or 1000 nucleotides which are able to hybridize to the active Botulinum toxin sequence under stringent hybridization conditions (such as the ones described by Sambrook et al., Molecular Cloning, Laboratory Manuel, Cold Spring, Harbor Laboratory Press, N.Y.).

Pharmaceutical Compositions, Methods of Administration and Combination Treatments

In some embodiments, a cholinergic antagonist, a cholinergic agonist, an ACh inhibitor, a NGF inhibitor, a TRK inhibitor, or a Botulinum toxin can be supplied in the form of a pharmaceutical composition, comprising an isotonic excipient prepared under sufficiently sterile conditions for human administration. Choice of the excipient and any accompanying elements of the composition comprising a cholinergic antagonist, a cholinergic agonist, an ACh inhibitor, a NGF inhibitor, a TRK inhibitor, or a Botulinum toxin will be adapted in accordance with the route and device used for administration. In some embodiments, a composition comprising a cholinergic antagonist, a cholinergic agonist, an ACh inhibitor, a NGF inhibitor, a TRK inhibitor, or a Botulinum toxin can also comprise, or be accompanied with, one or more other ingredients that facilitate the delivery or functional mobilization of the cholinergic antagonist, the cholinergic agonist, the ACh inhibitor, the NGF inhibitor, the TRK inhibitor, or Botulinum toxin.

These methods described herein are by no means all-inclusive, and further methods to suit the specific application is understood by the ordinary skilled artisan. Moreover, the effective amount of the compositions can be further approximated through analogy to compounds known to exert the desired effect.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A cholinergic antagonist (such as, e.g., darifenacin, scopolamine, amitriptyline, or a tricyclic antidepressant), a cholinergic agonist (such as, e.g. pilocarpine, aceclidine, arecoline, cevimeline, bethanechol, muscarine, oxotremorine, carbachol or methacholine), an ACh inhibitor, a NGF inhibitor, a TRK inhibitor, or a Botulinum toxin can be administered to the subject one time (e.g., as a single injection or deposition). Alternatively, a cholinergic antagonist, a cholinergic agonist, an ACh inhibitor, a NGF inhibitor, a TRK inhibitor, or a Botulinum toxin can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. Furthermore, a cholinergic antagonist, a cholinergic agonist, an ACh inhibitor, a NGF inhibitor, a TRK inhibitor, or a Botulinum toxin can be co-administrated with another therapeutic.

In one embodiment, a cholinergic antagonist, a Botulinum toxin, an ACh inhibitor, a NGF inhibitor, a TRK inhibitor, or a surgical denervation can be co-administrated with a cytotoxic therapy. In one embodiment, a cholinergic antagonist, a Botulinum toxin, an ACh inhibitor, a NGF inhibitor, a TRK inhibitor, or a surgical denervation can be co-administrated with a chemotherapy drug. In one embodiment, a cholinergic antagonist, a Botulinum toxin, an ACh inhibitor, a NGF inhibitor, a TRK inhibitor, or a surgical denervation can be co-administrated with 5-fluorouracil. In one embodiment, a cholinergic antagonist, a Botulinum toxin, an ACh inhibitor, a NGF inhibitor, a TRK inhibitor, or a surgical denervation can be co-administrated with oxaliplatin. In one embodiment, a cholinergic antagonist, a Botulinum toxin, an ACh inhibitor, a NGF inhibitor, a TRK inhibitor, or a surgical denervation can be co-administrated with 5-fluorouracil and oxaliplatin. Some non-limiting examples of conventional chemotherapy drugs include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, 5-fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, rituximab, testosterone, thioguanine, vincristine, vindesine, thiotepa, topotecan, tretinoin, vinblastine, trastuzumab, and vinorelbine.

In one embodiment, the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound.

A targeted anti-tumor compound is a drug designed to attack cancer cells more specifically than standard chemotherapy drugs can. Most of these compounds attack cells that harbor mutations of certain genes, or cells that overexpress copies of these genes.

An alkylating agent works directly on DNA to prevent the cancer cell from propagating. These agents are not specific to any particular phase of the cell cycle. In one embodiment, alkylating agents can be selected from busulfan, cisplatin, carboplatin, chlorambucil, cyclophosphamide, ifosfamide, dacarbazine (DTIC), mechlorethamine (nitrogen mustard), melphalan, and temozolomide.

An antimetabolite makes up the class of drugs that interfere with DNA and RNA synthesis. These agents work during the S phase of the cell cycle and are commonly used to treat leukemia, tumors of the breast, ovary, and the gastrointestinal tract, as well as other cancers. In one embodiment, an antimetabolite can be 5-fluorouracil, capecitabine, 6-mercaptopurine, methotrexate, gemcitabine, cytarabine (ara-C), fludarabine, or pemetrexed.

Topoisomerase inhibitors are drugs that interfere with the topoisomerase enzymes that are important in DNA replication. Some examples of topoisomerase I inhibitors include topotecan and irinotecan while some representative examples of topoisomerase II inhibitors include etoposide (VP-16) and teniposide.

Anthracyclines are chemotherapy drugs that also interfere with enzymes involved in DNA replication. These agents work in all phases of the cell cycle and thus, are widely used as a treatment for a variety of cancers. In one embodiment, an anthracycline used with respect to the invention can be daunorubicin, doxorubicin (Adriamycin), epirubicin, idarubicin, or mitoxantrone.

In one embodiment, a cholinergic antagonist, a Botulinum toxin, an ACh inhibitor, a NGF inhibitor, a TRK inhibitor, or a surgical denervation can be co-administrated with radiation therapy. Some non-limiting examples of conventional radiation therapy include: external beam radiation therapy, sealed source radiation therapy, unsealed source radiation therapy, particle therapy, and radioisotope therapy.

In one embodiment, a cholinergic antagonist, a Botulinum toxin, an ACh inhibitor, a NGF inhibitor, a TRK inhibitor, or a surgical denervation can be co-administrated with a cancer immunotherapy. Cancer immunotherapy comprises using the immune system of the subject to treat a cancer. For example, the immune system of a subject can be stimulated to recognize and eliminate cancer cells. Some non-limiting examples of cancer immunotherapy include: cancer vaccines, therapeutic antibodies, such as monoclonal antibody therapy (e.g., Bevacizumab, Cetuximab, Panitumumab), cell based immunotherapy, and adoptive cell based immunotherapy.

A cholinergic antagonist, Botulinum toxin, an ACh inhibitor, a NGF inhibitor, a TRK inhibitor, or surgical denervation may also be used in combination with surgical or other interventional treatment regimens used for the treatment of gastric cancer or colon cancer. In one embodiment, the surgical treatment is an endoscopic resection surgery. In one embodiment, the surgical treatment is a gastrectomy surgery. In one embodiment, a cholinergic antagonist, Botulinum toxin, an ACh inhibitor, a NGF inhibitor, a TRK inhibitor, or surgical denervation is administered or performed before, during, or after the endoscopic resection surgery or gastrectomy surgery is performed. Other surgical treatments for gastric cancer or colon cancer can be used and will be apparent to one of skill in the art.

A cholinergic antagonist, an ACh inhibitor, a NGF inhibitor, a TRK inhibitor, or a Botulinum toxin may be used in combination with surgical denervation or a surgical treatment of gastric cancer or colon cancer, such as, an endoscopic resection surgery or a gastrectomy surgery. In some embodiments the cholinergic antagonist, the ACh inhibitor, the NGF inhibitor, the TRK inhibitor, or Botulinum toxin can be administered during a perioperative period. In some embodiments, the perioperative period begins at the time of admission to a healthcare facility (e.g., a hospital) for a surgical procedure (e.g., cancer surgery), about 1 hour before admission, about 2 hours before admission, about 4 hours before admission, about 6 hours before admission, about 8 hours before admission, about 12 hours before admission, about 1 day before admission, about 2 days before admission, about 3 days before admission, about 4 days before admission, about 5 days before admission, about 6 days before admission, or about 1 week before admission. In some embodiments, the perioperative period ends at the time of release from an immediate post-care facility (e.g., a recovery room) after a surgical procedure, about 1 hour post-release, about 2 hours post-release, about 4 hours post-release, about 6 hours post-release, about 8 hours post-release, about 10 hours post-release, about 12 hours post-release, about 1 day post-release, about 2 days post-release, about 3 days post-release, about 4 days post-release, about 5 days post-release, about 6 days post-release, about 1 week post-release, about 2 weeks post-release, about 3 weeks post-release, or about 4 weeks post-release.

The compositions of this invention can be formulated and administered to reduce the symptoms associated with a disease of the digestive system by any means that produce contact of the active ingredient with the agent's site of action in the body of a human or non-human subject. For example, the compositions of this invention can be formulated and administered to reduce the symptoms associated with a digestive system cancer, or a dysplasia of the digestive system, or cause a decrease in cell proliferation, or a decrease in tumor growth. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the cholinergic antagonist, the ACh inhibitor, the NGF inhibitor, the TRK inhibitor, or Botulinum toxin in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art

A composition of the invention can be administered to a subject in need thereof. Subjects in need thereof can include but are not limited to, for example, a mammal such as a dog, a cat, a cow, a horse, a rabbit, a monkey, a pig, a sheep, a goat, or a human.

A composition of the invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the invention (e.g., that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the invention. Single unit dosage forms suitable for oral administration, such as, but not limited to, tablets, capsules, gel-caps, caplets, or powders, that are adapted for sustained release are encompassed by the invention.

The dosage administered can be a therapeutically effective amount of the composition sufficient to result in treatment of an a digestive system cancer (e.g. a gastric cancer, a colon cancer), a decrease in cell proliferation, a decrease in tumor growth, or treatment of dysplasia of the digestive system, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In one embodiment, a cholinergic antagonist, a cholinergic agonist, an ACh inhibitor, a NGF inhibitor, a TRK inhibitor, or a Botulinum toxin is administered at least once daily. In another embodiment, a cholinergic antagonist, a cholinergic agonist, an ACh inhibitor, a NGF inhibitor, a TRK inhibitor, or a Botulinum toxin is administered at least twice daily. In some embodiments, a cholinergic antagonist, a cholinergic agonist, an ACh inhibitor, a NGF inhibitor, a TRK inhibitor, or a Botulinum toxin for at least 1 week, for at least 2 weeks, for at least 3 weeks, for at least 4 weeks, for at least 5 weeks, for at least 6 weeks, for at least 8 weeks, for at least 10 weeks, for at least 12 weeks, for at least 18 weeks, for at least 24 weeks, for at least 36 weeks, for at least 48 weeks, or for at least 60 weeks. In further embodiments, a cholinergic antagonist, a cholinergic agonist, an ACh inhibitor, a NGF inhibitor, a TRK inhibitor, or a Botulinum toxin is administered in combination with a second therapeutic agent or with a surgical procedure.

Toxicity and therapeutic efficacy of therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD50/ED₅₀. Therapeutic agents that exhibit large therapeutic indices are useful. Therapeutic compositions that exhibit some toxic side effects can be used.

Experimental animals can be used as models for human disease. For example, mice can be used as a mammalian model system. The physiological systems that mammals possess can be found in mice, and in humans, for example. Certain diseases can be induced in mice by manipulating their environment, genome, or a combination of both. For example, the INS-GAS mouse model is a model for human gastric cancer. In another example, the carcinogen-induced [N-nitroso-N-methylurea (MNU)] mouse model is a model for human gastric cancer. In another example, the Helicobacter pylori (Hp)-infected H+/K+-ATPase (adenosine triphosphatase)-IL-1β(interleukin-1β) model is a model for human gastric cancer. In another example, TFF2-Cre x R26-LSL-NGF or H+K+ATPase-Cre x R26-LSL-NGF mice are a model for gastric cancer. In another example, Villin-Cre or K19-Cre x R26-LSL-NGF mice are a model for intestinal or colon tumorigenesis. Mist1-CreERT x R26-LSL-NGF are a model for pancreatic and gastric tumorigenesis. In addition, there are numerous other genetically engineered models of cancer.

Administration of a cholinergic antagonist, a cholinergic agonist, an ACh inhibitor, a NGF inhibitor, a TRK inhibitor, or a Botulinum toxin is not restricted to a single route, but may encompass administration by multiple routes. Multiple administrations may be sequential or concurrent. Other modes of application by multiple routes will be apparent to one of skill in the art.

### EXAMPLES

The following examples illustrate the present invention, and are set forth to aid in the understanding of the invention, and should not be construed to limit in any way the scope of the invention as defined in the statements of the invention which follow thereafter.

The Examples described below are provided to illustrate aspects of the present invention and are not included for the purpose of limiting the invention.

### Example 1

### Denervation Suppresses Gastric Tumorigenesis

The nervous system plays an important role in the regulation of epithelial homeostasis and has also been postulated to play a role in tumorigenesis. Provided herein is evidence that proper innervation is critical at all stages of gastric tumorigenesis. In three separate mouse models of gastric cancer, surgical or pharmacological denervation of the stomach (bilateral or unilateral truncal vagotomy, or local injection of botulinum toxin) markedly reduced tumor incidence and progression, but only in the denervated portion of the stomach. Vagotomy or botulinum toxin treatment also enhanced the therapeutic effects of systemic chemotherapy and prolonged survival. Denervation induced suppression of tumorigenesis was associated with inhibition of Wnt signaling and suppression of stem cell expansion. In gastric organoid cultures, neurons stimulated growth in a Wnt-mediated fashion through cholinergic signaling. Furthermore, pharmacological inhibition or genetic knockout of the muscarinic acetylcholine M3 receptor suppressed gastric tumorigenesis. In gastric cancer patients, tumor stage correlated with neural density and activated Wnt signaling, whereas vagotomy reduced the risk of gastric cancer. Together, these findings suggest that vagal innervation contributes to gastric tumorigenesis via M3 receptor—mediated Wnt signaling in the stem cells, and that denervation might represent a feasible strategy for the control of gastric cancer.

Introduction

The nervous system regulates epithelial homeostasis in different ways, and this regulation by the nervous system partly involves modulation of stem and progenitor cells (1, 2). There is also crosstalk between tumor cells and nerves, such that tumors induce active neurogenesis, resulting in increased neuronal density in preneoplastic and neoplastic tissues (3-6). In addition, activation of muscarinic receptors has been shown to promote cell transformation and cancer progression (3-6). A recent study demonstrated that prostate tumors are infiltrated by autonomic nerves contributing to cancer development and dissemination (7). Given the potential ability of nerves to influence gut stem and progenitor cells, and the prevailing notion that persistently elevated gut epithelial proliferation predisposes to cancer formation, it is believed that axonal reflexes could also modulate the conversion of stem or progenitor cells into cancer cells (8, 9).

Gastric cancer is the fifth most common cancer and the third leading cause of cancer mortality worldwide, with a 5-year survival rate of less than 25% (10, 11). It has been demonstrated that vagotomy decreases gastric mucosal thickness and cellular proliferation (12, 13). An epidemiological study showed that the risk of gastric cancer [standardized incidence ratio (SIR)] after vagotomy was not reduced during the first 10-year period, but was reduced by 50% (SIR 0.5) during the second 10-year follow-up (14, 15). Provided herein is evidence that proper innervation is critical for gastric tumorigenesis, and suggest that nerves may represent a therapeutic target for the treatment of gastric cancer.

Results

Gastric lesser curvature has high vagal innervation and high incidence of tumors.

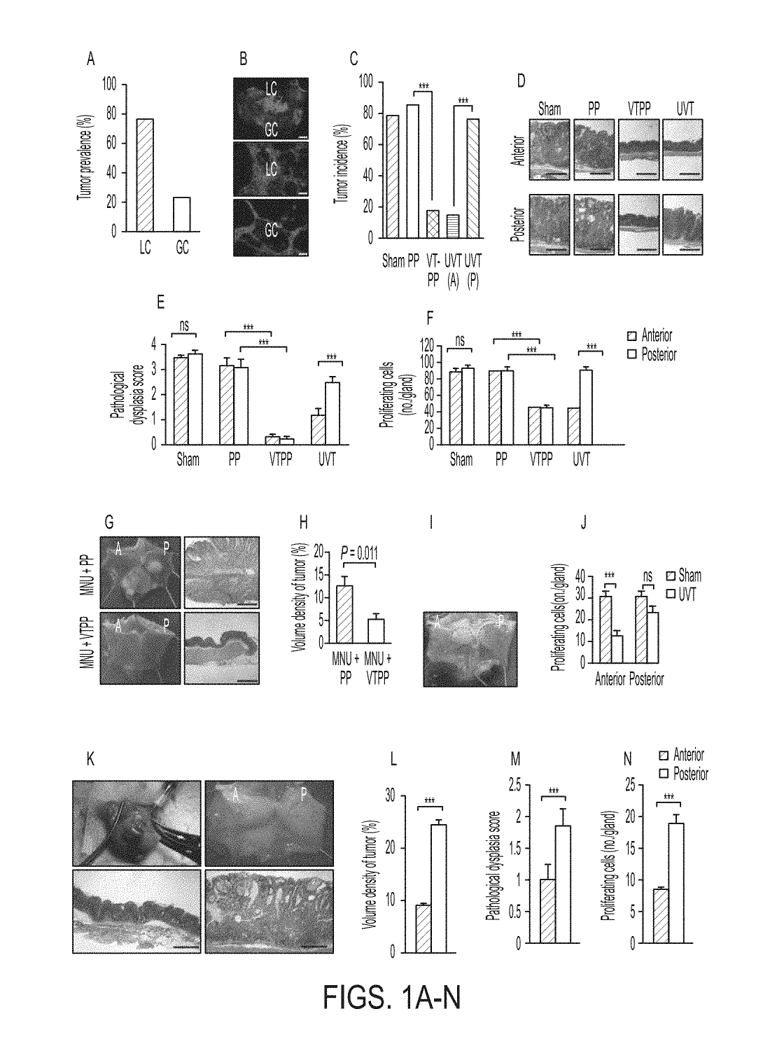

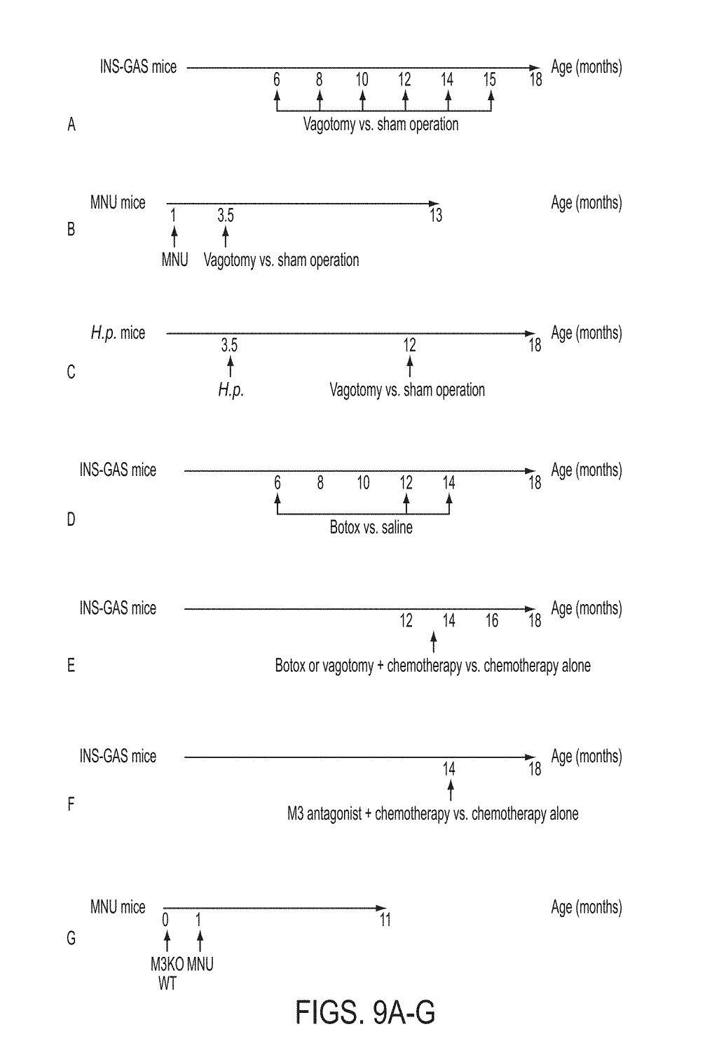

In humans, there is a higher incidence of gastric cancer in the lesser (˜80% of tumors) than the greater curvature (16, 17). This distribution was also observed in the INS-GAS mouse model, a genetic mouse model of spontaneous gastric cancer (18, 19), in which there was a similar prevalence (77%) of tumors in the lesser curvature (FIG. 1A). INS-GAS mice F1 do not display obvious preneoplastic lesions until 6 months of age, but afterward, they develop gastric cancer through stages of atrophy, metaplasia, and, finally, dysplasia at 12 months of age (18, 19). Topographic analysis of vagus nerve fibers and terminals in the murine stomach revealed a higher density of neurons and larger ganglia in the lesser curvature compared to the greater curvature (FIG. 1B), correlating with the observed pattern of tumor formation. This possible association between the distribution of vagal nerve fibers and the appearance of gastric tumors in INS-GAS mice prompted us to study the role of nerves in gastric tumorigenesis (FIG. 9 and Table 1).

Surgical Denervation at Preneoplastic Stage Attenuates Tumorigenesis in Mouse Models of Gastric Cancer

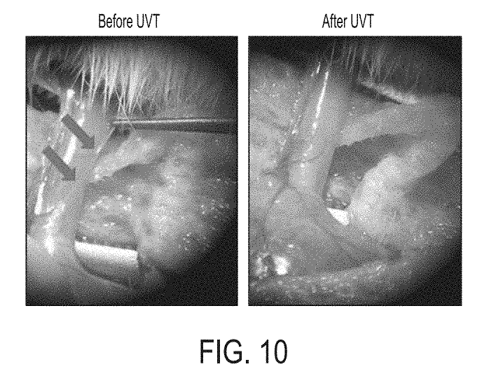

In the first set of experiments, vagotomy was performed in INS-GAS mice at 6 months of age. Subsequently, the effects of vagotomy were examined 6 months after surgery. One hundred seven INS-GAS mice were subjected to either subdiaphragmatic VTPP, UVT (FIG. 10), sham operation, or PP. The unilateral vagotomy approach takes advantage of the fact that each (anterior or posterior) vagal trunk innervates only one-half of the stomach. Consequently, denervation of one side of the stomach does not impair the overall functional capacity of the stomach, leaving gastric acid output, circulating gastrin levels, and gastric motility unchanged (13, 20).

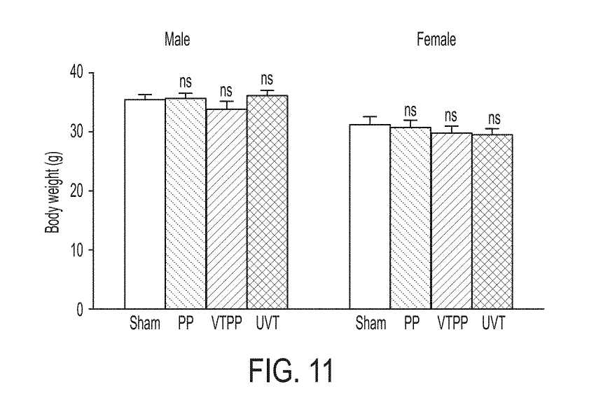

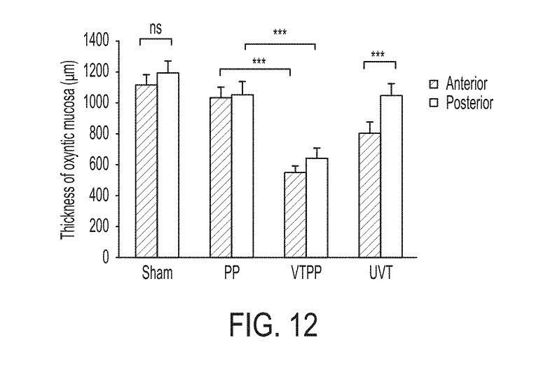

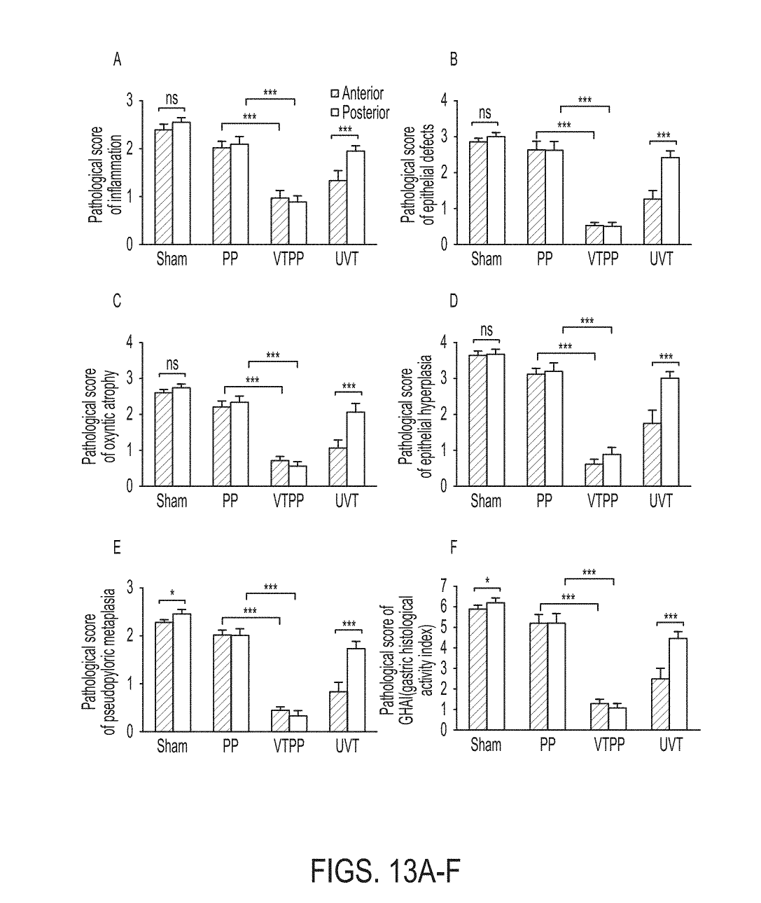

Six months after surgery, body weight was unchanged in either male or female mice (FIG. 11). Tumor incidence was 17% after VTPP versus 86% after PP alone, 14% in the anterior side versus 76% in the posterior side after anterior UVT, and 78% in the sham-operated mice (FIG. 1C). Histological examination revealed the reduction of mucosal thickness after VTPP (compared to PP) or UVT (compared to the corresponding posterior side) (FIG. 1D and FIG. 12), indicating successful denervation (12, 13). Pathological evaluation (21) revealed that vagotomy attenuated the score for dysplasia and reduced the number of proliferating cells (FIGS. 1,E and F) and the scores for inflammation, epithelial defects, oxyntic atrophy, epithelial hyperplasia, pseudopyloric metaplasia, and gastric histological activity index (GHAI) (FIG. 13,A to F).

To confirm these findings, the surgical approach was tested in two other mouse models of gastric cancer, namely, the carcinogeninduced [N-nitroso-N-methylurea (MNU)] (22) and the Helicobacter pylori (Hp)-infected H+/K+-ATPase (adenosine triphosphatase)-IL-1b (interleukin-1b) mousemodels (23). In the MNU model, VTPP performed 1 week after completion of MNU treatment inhibited tumor development at 13 months of age (FIGS. 1,G and H). Infection of H+/K+-ATPase-IL-1b mice with Hp accelerated gastric tumorigenesis, resembling Hp-related atrophy-metaplasia-dysplasia sequence in humans. UVT performed 8.5 months after Hp infection (at 12 months of age) reduced tumor size and number of proliferating cells in the denervated side of the stomach at 18 months of age (FIGS. 1,I and J). Thus, the findings from these three independent models demonstrate the importance of functional innervation in gastric tumorigenesis.

Pharmacological Denervation at an Early Preneoplastic Stage Attenuates Gastric Tumorigenesis.

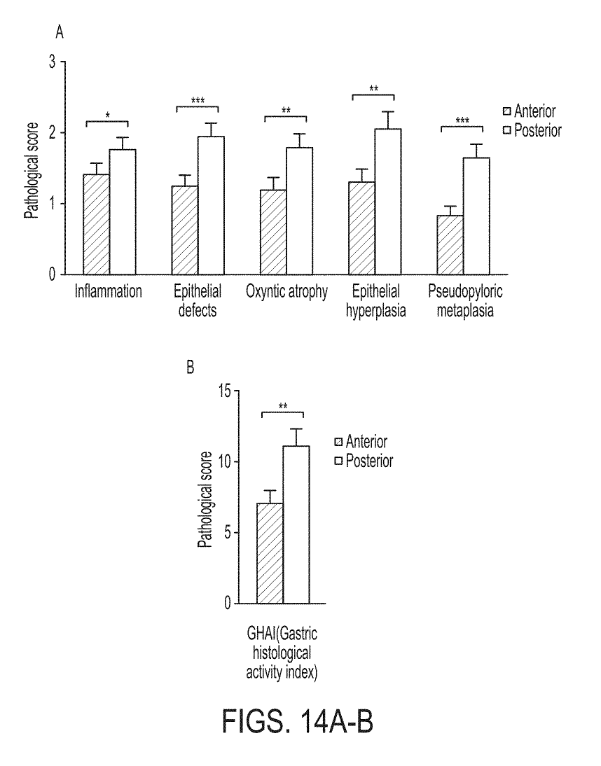

To prove that the effects of surgical denervation were primarily local (acting at vagus nerve terminals within the gastric mucosa), unilateral injection of botulinum toxin A (Botox) into the gastric wall was performed in INS-GAS mice at 6 months of age. Botox enters into the axon terminal through vesicle internalization and cleaves synaptosomal-associated protein 25, leading to impaired exocytosis of neurotransmitters, including acetylcholine (24). Botox was injected subserosally along the greater curvature in the anterior side of the stomach (FIG. 1K). Six months later, tumor size, score for dysplasia, and number of proliferating cells were markedly reduced in the anterior wall compared with the posterior side of the stomach (FIGS. 1,L to N). Moreover, these changes were associated with attenuated scores for inflammation, epithelial defects, atrophy, hyperplasia, pseudopyloric metaplasia, and GHAI (FIG. 14,A and B). Hence, these findings confirm an important role of local signaling from vagus nerve endings in early gastric tumorigenesis.

Surgical or Pharmacological Denervation Attenuates Gastric Tumor Progression.

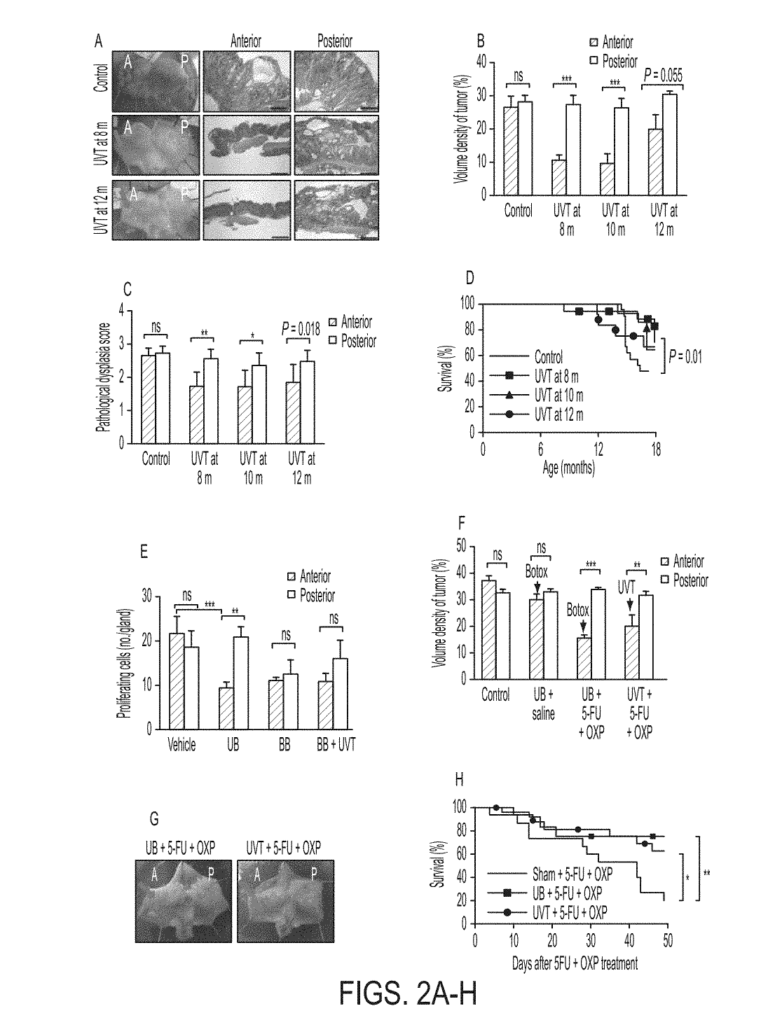

Because vagotomy or Botox treatment had a protective effect at preneoplastic stages, whether gastric denervation could also inhibit tumor progression at later stages was examined. INS-GAS mice at 8, 10, or 12 months of age were subjected to anterior UVT and euthanized at 18 months of age. In these mice, the tumors were smaller with less severe dysplasia in the anterior side compared to the posterior side of the stomach, suggesting that denervation inhibits tumor progression in mice with established neoplastic changes (FIG. 2,A to C).

Whereas the average life span of wild-type FVB/N mice is well over 24 months, the survival of INS-GAS mice at 18 months of age was 53% (16 of 30 mice). Attenuation of tumor burden by UVT improved the 18-month survival when compared to age-matched INS-GAS mice: 71% when UVT was performed at 8 months, 64% when performed at 10 months, and 67% when performed at 12 months, respectively (FIG. 2D). Next, INS-GAS mice at 12 months of age were subjected to vehicle or Botox treatment unilaterally or bilaterally, with or without UVT. The tumor cell proliferation was reduced in the anterior side of the stomach where Botox was injected, when compared to the posterior side or vehicle-treated anterior side (FIG. 2E). The combination of UVT and Botox did not further reduce cellular proliferation, indicating that vagotomy and Botox likely act through the same mechanism. These results further suggest that surgical or pharmacological denervation inhibits gastric cancer progression even when applied at later stages of gastric tumorigenesis.

Denervation Enhances the Effect of Chemotherapy in the Treatment of Gastric Cancer

Next, whether denervation could enhance the effects of systemic chemotherapy in invasive gastric cancer was examined. INS-GAS mice at 12 to 14 months of age received systemic administration of 5-FU+oxaliplatin or saline along with unilateral Botox treatment or UVT. The experiment was designed such that the nondenervated half of the stomach in each animal served as an internal control, either a chemotherapy-only control (the posterior side of the stomach in two of the groups received chemotherapy alone) or an untreated control. An additional group of INS-GAS mice was included as untreated controls. As early as 2 months after starting treatment, tumor size was reduced in mice treated with chemotherapy, specifically in the denervated areas of the stomach (the anterior side) after unilateral vagotomy or Botox injection (FIG. 2,F and G). The combination of either Botox or UVT with chemotherapy increased survival compared to chemotherapy alone (FIG. 2H). Together, these findings suggest that the combination of denervation and chemotherapy has an enhanced effect on tumor growth and survival.

Denervation Inhibits Gastric Wnt Signaling and Suppresses Stem Cell Expansion through the M3 Receptor.

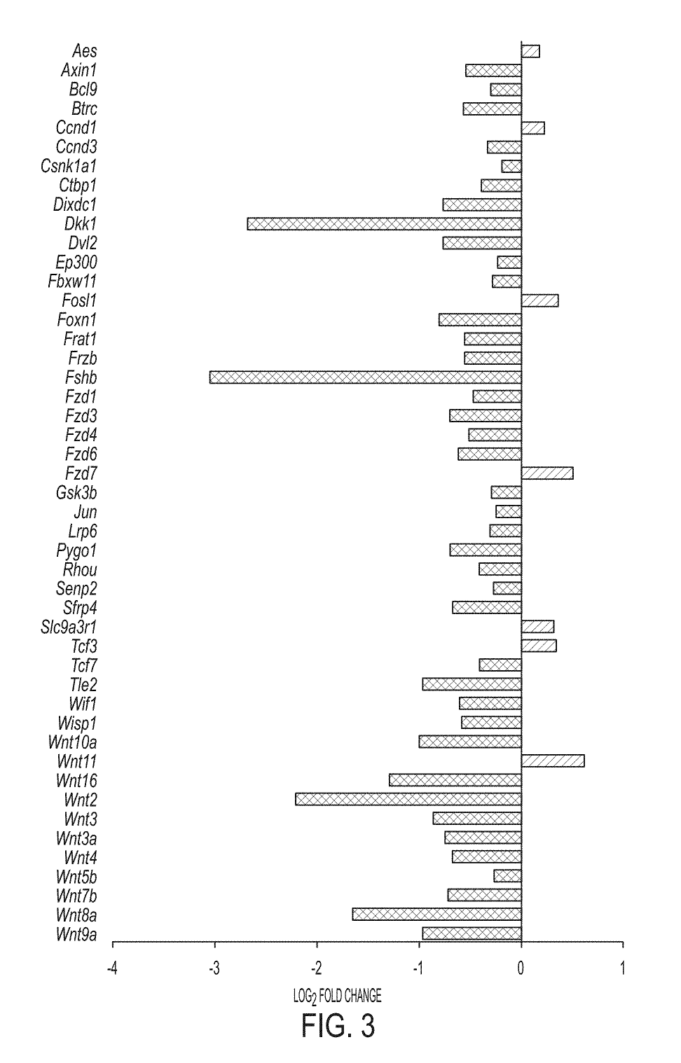

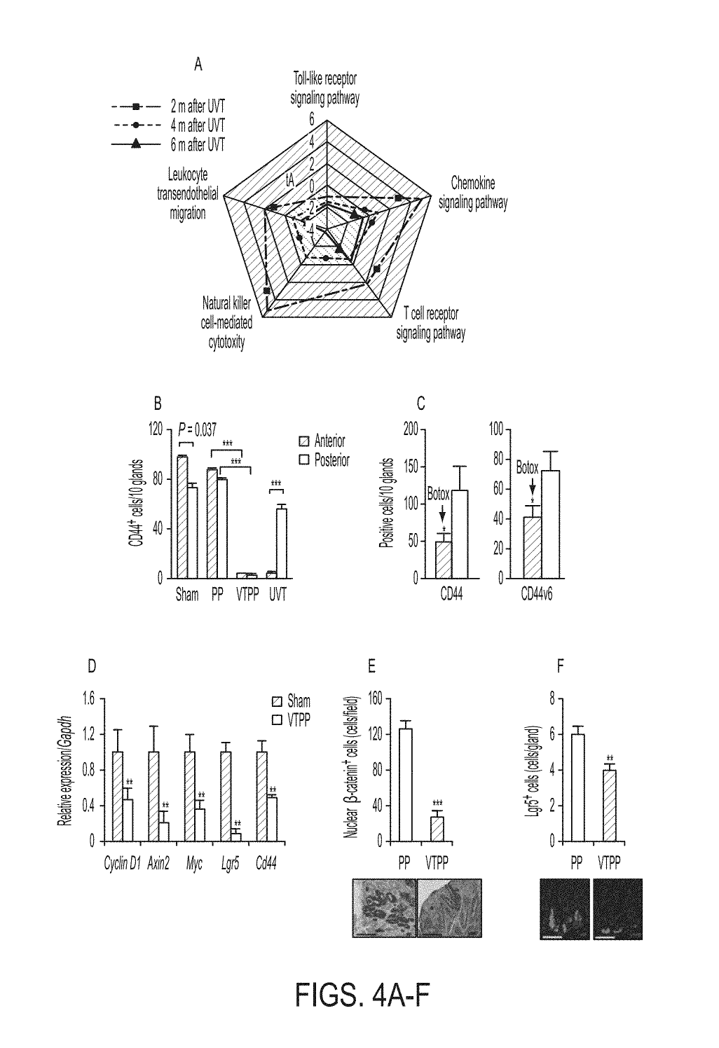

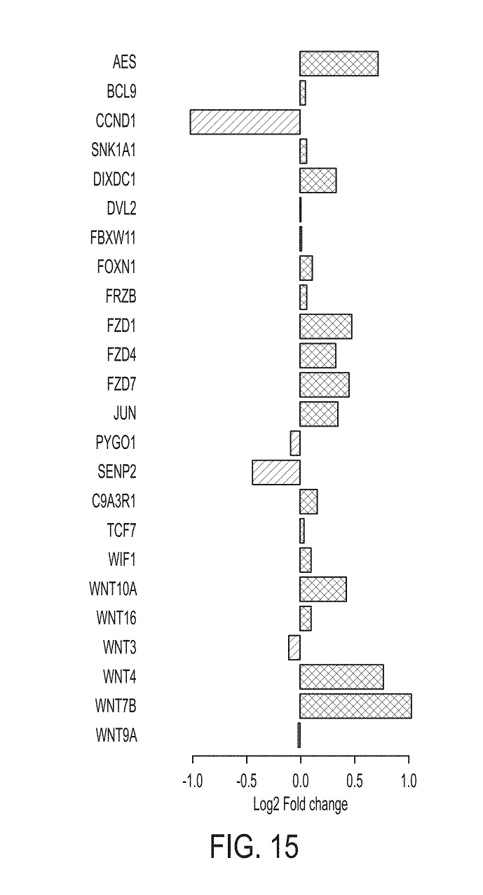

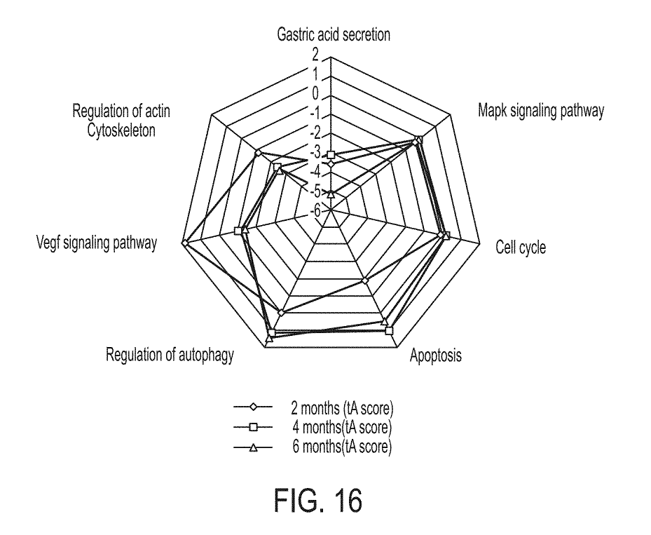

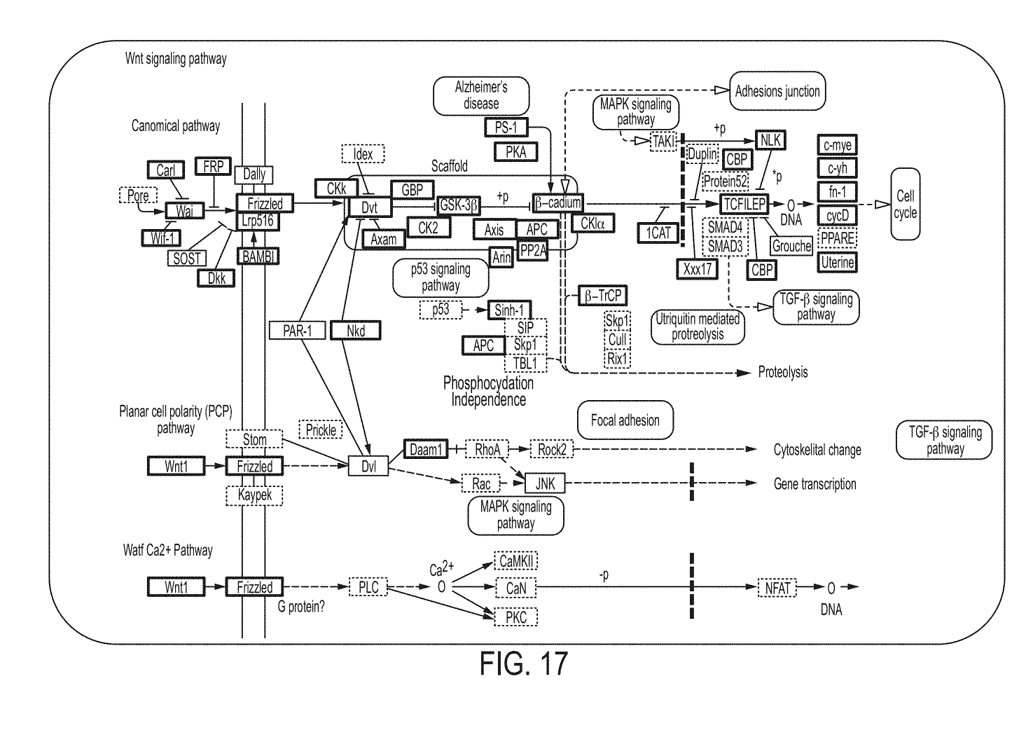

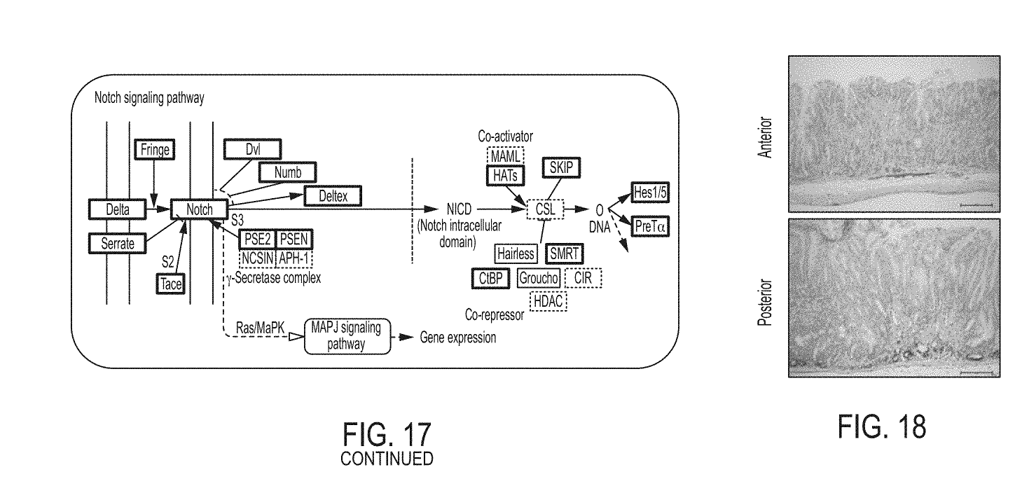

Gene expression profiling was performed in INS-GAS mice versus wild-type mice, and in unilaterally vagotomized INS-GAS mice (UVT performed at 6 months of age). Comparison between INS-GAS mice and wild-type mice showed up-regulation of the Wnt signaling pathway in INS-GAS mice (FIG. 15). Comparison between the vagotomized anterior and the untreated posterior side of the same stomach revealed many differentially expressed KEGG pathways, including those involved in gastric acid secretion, mitogen-activated protein kinase signaling, cell cycle, apoptosis, autophagy, vascular endothelial growth factor signaling, and actin cytoskeleton (FIG. 16). The Wnt and Notch signaling pathways were markedly inhibited in the vagotomized side [validated by quantitative reverse transcription polymerase chain reaction (qRT-PCR) arrays] (FIG. 3 and FIG. 17). The inhibition of Wnt signaling was persistent at 2, 6, 8, and 10 months after vagotomy [microarray data deposited in the Gene Expression Omnibus (GEO) database, accession no. GSE30295]. Inflammation-related pathways, including T cell receptor signaling, natural killer cell—mediated cytotoxicity, leukocyte transendothelial migration, and chemokine signaling, were activated at 2 months, but then inhibited at 4 and 6 months after vagotomy, whereas Toll-like receptor signaling was F4 inhibited at all the time points (FIG. 4A).

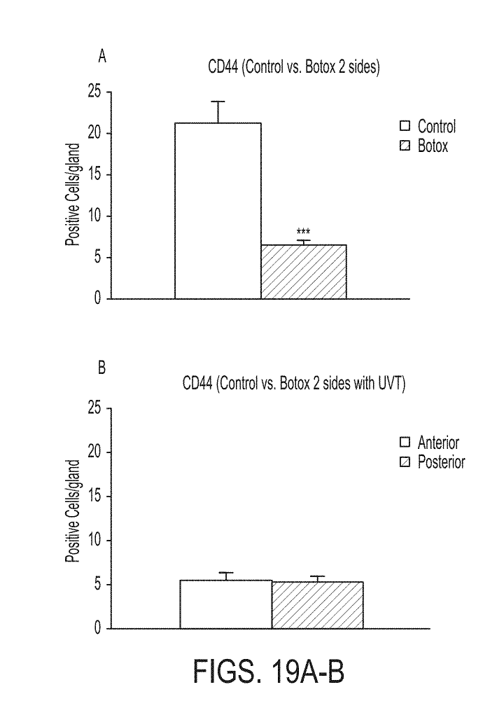

The Wnt signaling pathway is a major regulator of gastrointestinal stem cells and tumorigenesis (25, 26). CD44 is a known target of the Wnt signaling pathway (27) and has been shown to label a cancer initiating cell population (28). Lgr5 is a marker of gastric stem cells in normal as well as cancer tissues, and also a target of the Wnt signaling pathway (29). Either vagotomy (VTPP and UVT) or Botox treatment induced down-regulation of CD44 (and CD44v6) in the gastric mucosa of INS-GAS mice, although the combination of vagotomy and Botox did not lead to a further decrease in CD44 expression (FIG. 4,B and C, and FIGS. 18 and 19). Vagotomy also reduced the expression of Wnt target genes, such as Cyclin D1, Axin2, Myc, Lgr5, and Cd44, in MNU-treated mice (FIG. 4D). The number of cells with nu-AQ4 clear translocation of b-catenin and the number of Lgr5+ cells in MNU-treated mice were reduced after vagotomy (FIG. 4,E and F). These results suggest that disruption of neuronal signaling inhibits Wnt signaling and thereby stem cell expansion, resulting in the suppression of tumor development in both INS-GAS and MNU mouse models.

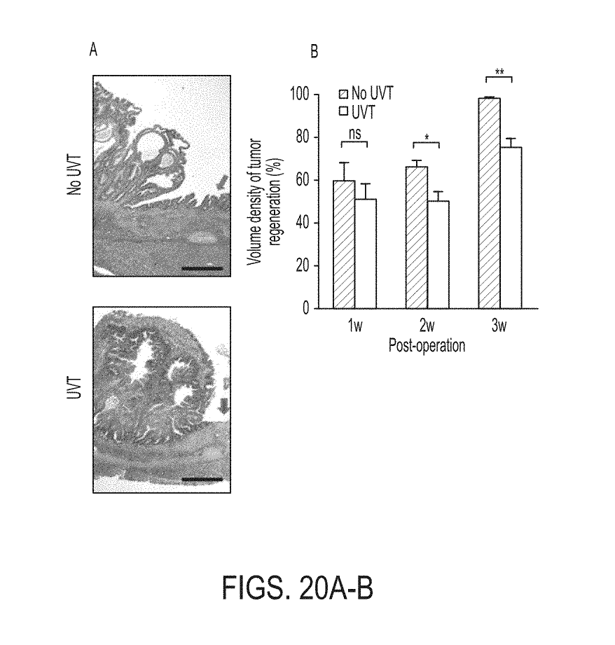

Wnt signaling is also known to be involved in tumor regeneration (30). A mouse model of tumor regeneration through topical application of acetic acid in INS-GAS mice has been established (31). In this model, vagotomy delayed tumor regeneration in the denervated side of the stomach (FIG. 20).

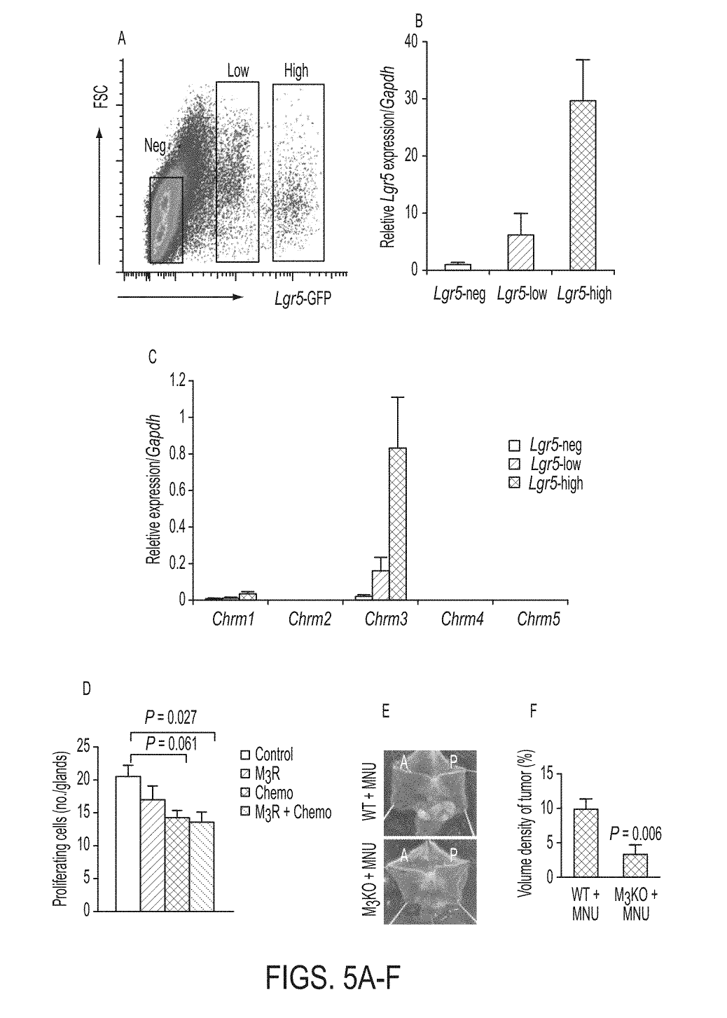

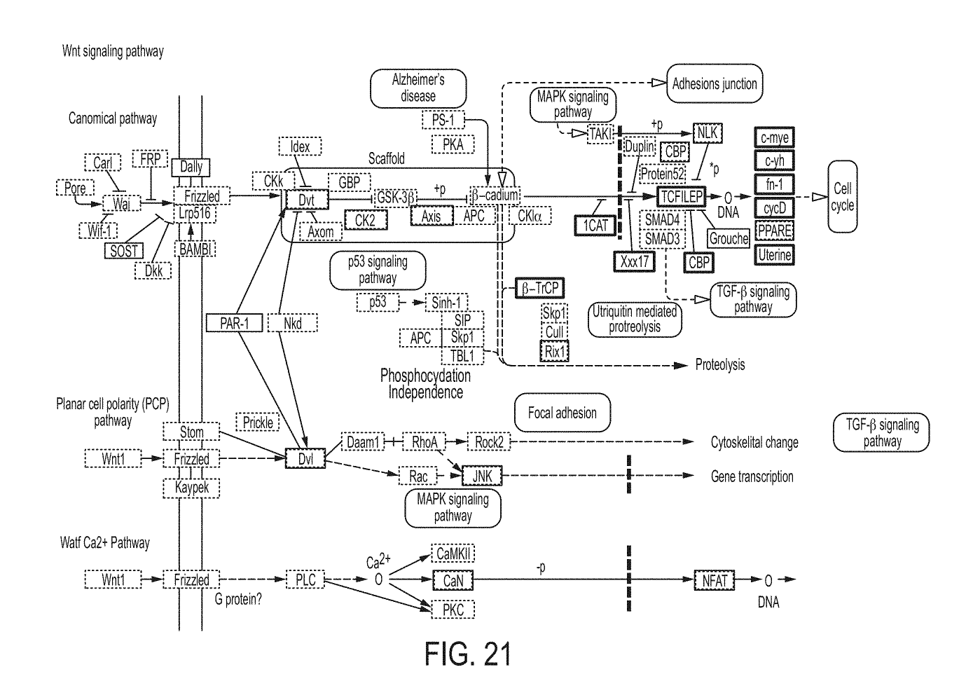

Next, whether vagotomy down-regulated Lgr5 expression via the muscarinic acetylcholine receptor 3 (M3R) was examined. Gastric epithelial cells from Lgr5-GFP mice were sorted on the basis of green fluorescent protein (GFP) expression. Afterward, Chrm3 gene expression was tested in Lgr5-negative, Lgr5-low, and Lgr5-high cell populations (FIG. 5A). There was coexpression of Lgr5 and F5 Chrm3 in the sorted cells from Lgr5-GFP mouse stomach, but other subtypes of muscarinic receptor were little expressed in those cells (FIG. 5,B and C), suggesting that Lgr5+ stem cell function may be modulated by muscarinic signaling. To investigate the involvement of M3 receptors in gastric tumorigenesis, INS-GAS mice were treated by continuous infusion of the specific M3 receptor antagonist, darifenacin (32), in combination with chemotherapy. Using an experimental design similar to that of the Botox and vagotomy experiment, it was found that the combination of darifenacin and chemotherapy reduced cellular proliferation of the tumors (FIG. 5D). Furthermore, the Wnt signaling pathway was analyzed in M3KO versus wild-type mice and found that several key genes, including one encoding b-catenin, were downregulated (FIG. 21). M3KO and wild-type mice were then exposed to MNU treatment. At 7.5 months after MNU treatment (11 months of age), M3KO mice had less tumor induction (57.1% versus 100%) and smaller tumor size when compared to wild-type controls (FIG. 5,E and F). Thus, the vagus nerve regulates gastric tumorigenesis at least in part through M3 receptor—mediated Wnt signaling, which is operative in Lgr5+ stem cells.

Neurons Activate Wnt Signaling in Gastric Stem cells through M3 Receptor.

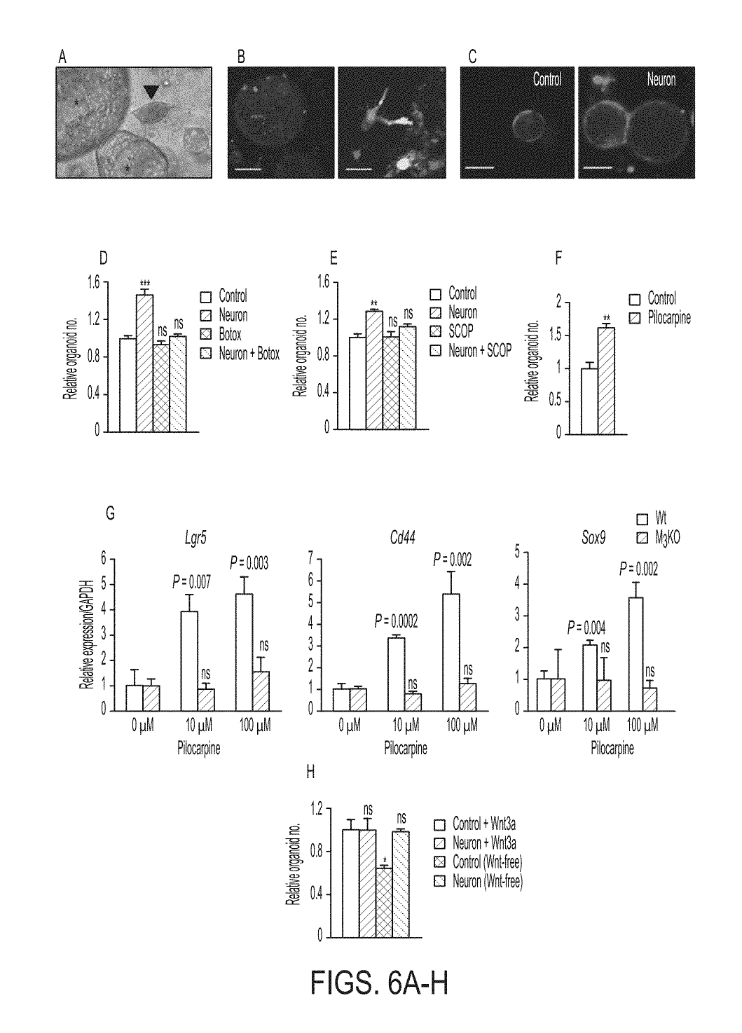

To demonstrate the potential regulatory role of nerves in the maintenance of gastric epithelium, an established in vitro culture system was used for gastric organoids (9). Primary neurons were isolated from murine spinal cord or the enteric nervous system of guinea pigs, and co-cultured with gastric glands (9, 33, 34). In culture, neurons showed outgrowth of neurites and evidence of direct contact with the gastric organoids (FIG. 6,A to C). Furthermore, co-culture with neuronsmarkedly F6 promoted gastric organoid growth (FIG. 6,D and E). The addition of either Botox or scopolamine (an unspecific muscarinic receptor antagonist) inhibited this stimulatory effect (FIG. 6,D and E), whereas pilocarpine (an unspecific muscarinic receptor agonist) stimulated organoid growth (FIG. 6F). Pilocarpine caused up-regulation of the gastric stem cell markers and Wnt target genes Lgr5, Cd44, and Sox9 (9) in a dosedependent manner. However, in gastric organoids of M3KO mice, pilocarpine showed no effects on the expression of these genes (FIG. 6G), highlighting the importance of the M3 receptor for stem cell expansion. Furthermore, co-culture with neurons could substitute for Wnt3a in gastric organoid cultures that are otherwise strictly dependent on addition of Wnt ligands (35) (FIG. 6H), confirming the ability of cholinergic signaling to induce ligand independent Wnt signaling in this in vitro system.

Gastric Cancer Patients Display Dysregulation of Wnt Signaling and Innervation in the Tumors.

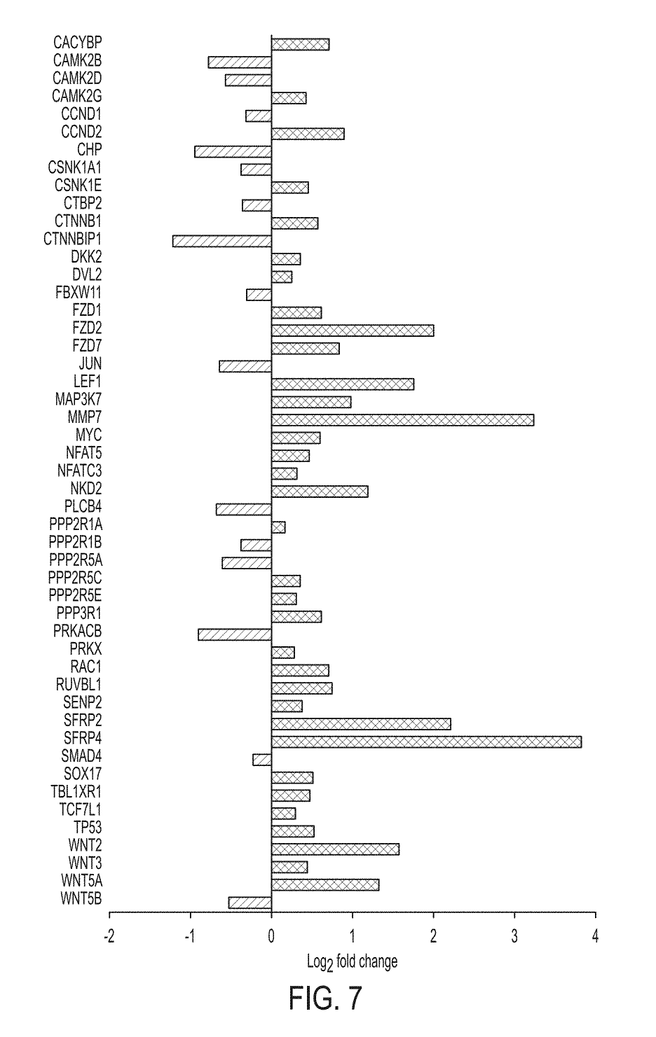

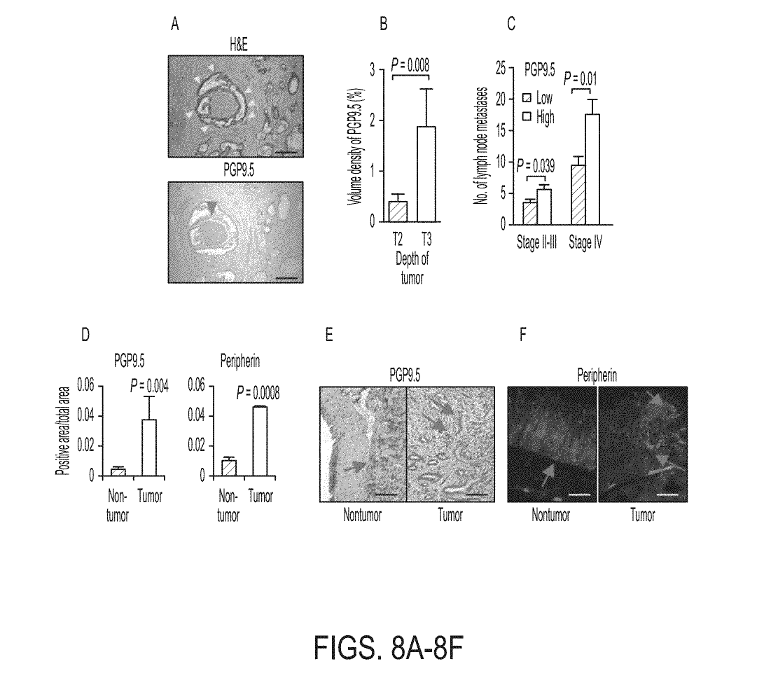

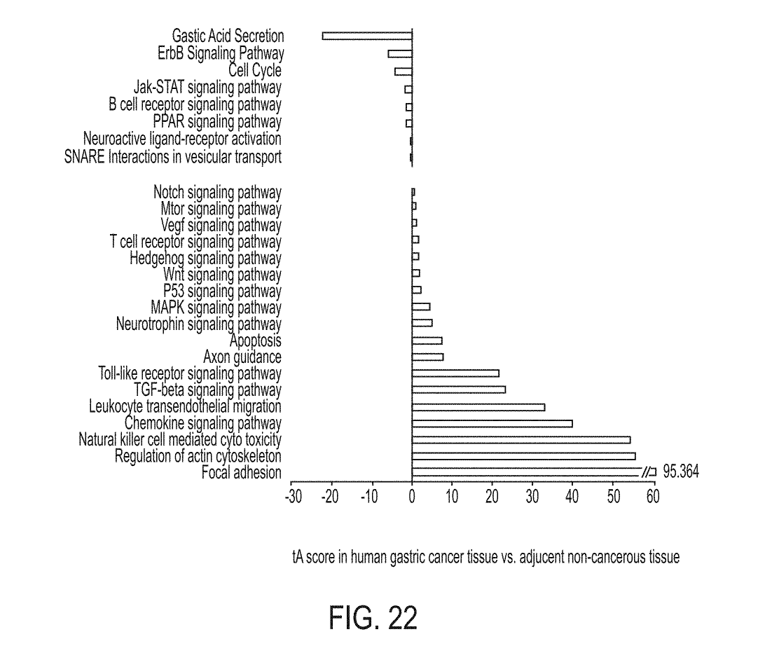

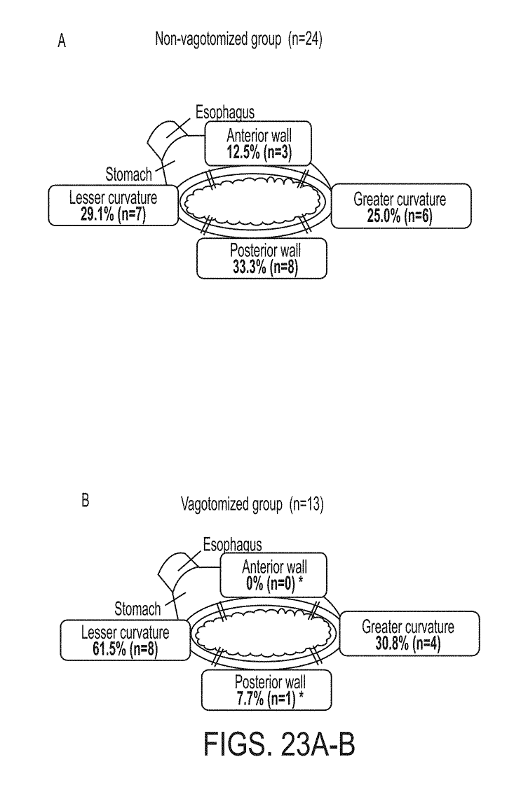

To further investigate the involvement of Wnt signaling, innervation, and gastric cancer progression in humans, three separate cohort studies of gastric cancer patients were evaluated (Table 2). In tumors from 17 primary gastric cancer patients, Wnt signaling, neurotrophin signaling, and axonal guidance pathways (along with other pathways) were activated in cancerous tissue when compared to adjacent noncancerous tissue (FIG. 7 and FIG. 22). In another F7 group of 120 primary gastric cancers, neuronal density was correlated with more advanced tumor stages (FIG. 8,A to C). A F8 similar increase in neuronal density was confirmed in tumors of mice treated with MNU (FIG. 8,D to F). In the third cohort of 37 patients, who developed gastric stump cancer after distal gastrectomy with or without vagotomy, 35% (13 of 37) of patients had undergone vagotomy. Of those 13 patients, only 1 had a tumor in the posterior wall and none had tumors in the anterior wall. In the 24 patients without vagotomy, tumors were observed in both anterior and posterior walls (FIG. 23).

Discussion

The results of the present study, using three independent mouse models of gastric cancer, demonstrate that either surgical or pharmacological denervation suppresses gastric tumorigenesis. The effect takes place primarily on terminal and intramucosal vagal branches, as shown by the response to unilateral vagotomy and localized Botox injection. Denervation therapy was effective in both early preneoplasia and late neoplasia/dysplasia, and it enhanced the effect of chemotherapy and prolonged survival in mice with advanced tumors. Gene expression and immunohistochemical analysis of stem cell markers, along with the in vitro gastric organoid test, revealed that cholinergic nerves directly modulate epithelial stem cells through activation of Wnt signaling via the M3 receptor. Analysis of human patients with gastric cancer also showed correlations between neural pathways and Wnt signaling and increased innervation in more advanced tumors, with decreased tumor risk in vagotomized stomach.

In contrast to the current results, previous vagotomy studies in rat models of chemically induced gastric cancer did not reveal an inhibitory effect (12, 36, 37). This is likely due to the earlier approach of bilateral vagotomy without pyloroplasty, which delayed gastric emptying and therefore increased the exposure time of orally administered chemical carcinogens on the gastric mucosa. To ensure that dose and time of MNU exposure were equalized in all the groups and to prevent retention of gastric contents, bilateral vagotomy with pyloroplasty or PP (as control) were performed after completion of the MNU dosing protocol, allowing analysis of the specific effects of the vagus nerve on the gastric mucosa. Thus, it was found that vagotomy inhibited gastric tumorigenesis in the MNU model. The data from two different genetically engineered mouse models of gastric cancer further established the inhibitory effect of denervation against gastric tumorigenesis. Given the limited availability of metastatic models of gastric cancer, the effect of denervation in metastatic lymph nodes or other organs remains unclear and needs to be further investigated in suitable models.

Previous studies suggested that nerves contribute to the normal stem cell niche (1, 2, 38), and a recent report has linked sympathetic nerves to prostate cancer progression (7). However, the stomach differs from other solid organs in that its autonomic innervation is largely parasympathetic in nature, and cholinergic nerves have been shown to regulate gastrointestinal proliferation (39). The present study demonstrated that Lgr5+ gastric stem cells express the M3 receptor, and that Wnt signaling in those cells is directly activated by cholinergic vagus stimulation, resulting in epithelial proliferation and stem cell expansion. Gastrointestinal stem cells are supported by a number of niche cells including Paneth cells, mesenchymal stem cells, myofibroblasts, smooth muscle cells, lymph and vascular endothelial cells, and bone marrow—derived stromal cells (40-42). Here, nerves regulating gastric stem cell expansion were identified during the tumorigenesis.

The vagus nerve has been shown to stimulate cell proliferation in the brain, liver, and stomach through the M3 receptor (43-45). Furthermore, activation of muscarinic receptors in cancer cells leads to enhanced Wnt signaling independent of Wnt ligands (46), and M3 receptor signaling has been implicated in the pathogenesis of intestinal neoplasia (6, 47, 48). Consistent with those findings, the present study demonstrates that genetic knockout or pharmacological inhibition of M3 receptor suppresses gastric tumor progression, pointing to the M3 receptor as a potential target for gastric cancer therapy. The M3 receptor antagonist darifenacin is already in clinical use for overactive urinary bladder (49) and has been shown to inhibit growth of small cell lung cancer xenografts (50). Given that chemotherapeutic agents and darifenacin appear to show cooperative effects, M3 receptor—targeting therapy combined with chemotherapy in unresectable gastric cancer patients could be considered in future trials, although further studies are needed to evaluate the safety and long-term effects of those regimens.

Canonical Wnt signaling controls epithelial homeostasis in the intestine and the stomach, and is thought to play a role in a subset of gastric cancers (9, 51). Here, it was shown that Wnt signaling is upregulated in the tumorigenic stomach and is down-regulated after vagotomy, suggesting that vagus nerve is a critical regulator of Wnt signaling in gastric tumorigenesis. Furthermore, gastric Wnt signaling was down-regulated in M3KO mice, which were resistant to MNU-induced tumorigenesis. In addition, inhibition of Notch signaling was also observed after vagotomy, which is in line with both Wnt and Notch signaling promoting the initiation of intestinal tumors (52, 53). Therefore, therapeutic modulation of Wnt signaling blockade using tankyrase inhibitors could also be considered, although the dose-limiting toxicity of available agents has restricted their clinical use to this point (54). Finally, a role for additional pathways (for example, prostaglandin E2 pathway) that may be modulated by nerves in gastric tumorigenesis cannot be excluded.

The finding that nerves play an important role in cancer initiation and progression highlights a component of the tumor microenvironment contributing to the cancer stem cell niche. The data strongly support the notion that denervation and cholinergic antagonism, in combination with other therapies, could represent a viable approach for the treatment of gastric cancer and possibly other solid malignancies.

Materials and Methods

581 mice divided into 14 experimental groups were used. In each experiment, mice were randomly divided into different subgroups (FIG. 9 and Table 1). INS-GAS mice with spontaneous gastric cancer were used as previously described (18, 19). Denervation was achieved by subdiaphragmatic bilateral truncal vagotomy, unilateral vagotomy, or Botox local injection. The tumor prevalence/incidence, tumor size, tumor regeneration, pathological changes, gene expression profiles, and immunohistochemical biomarkers were examined after denervation. In vitro gastric organoid culture was performed as described previously (9). Three cohort studies of human primary gastric cancer and gastric stump cancer were also performed, as well as gene expression profiling and KEGG pathway analysis. All studies and procedures involving animals and human subjects were approved by the Norwegian National Animal Research Authority, the Columbia University Institutional Animal Care and Use Committee, Gifu University, and the National Cancer Center Hospital East, Japan. Statistical comparisons were performed between experimental groups, between the anterior and posterior sides of the stomachs, and between groups of patients.

Animals

The insulin-gastrin (INS-GAS) transgenic mice were generated by Dr. T. C. Wang (18). Animals were further bred through sibling mating for more than 20 generations. 829 INS-GAS mice have been examined during the past 9 years. The percentage of the mice without preneoplastic lesions was 3.7% at 6 months of age, and the incidence rate of spontaneous gastric corpus cancer increased from 75% (at the beginning of this study in Jan. 2005) to 100% (May 2013) at 12 months of age without an additional infection with Helicobacter pylori. 187 INS-GAS mice were examined at 1 to 20 months of age during 2005, and 61 mice were found to have the gastric tumor after 9 months of age (see FIG. 1A: the tumor incidence at the lesser or greater curvature of the stomach). During 2012, 139 INS-GAS mice were examined at 12 months of age and all mice had the gastric tumor. M3KO mice were obtained from Dr. Koji Takeuchi at Kyoto Pharmaceutical University (45). M3KO mice had higher water intake than age- and sex-matched wild type mice (14.62±1.71 vs. 6.48±1.00 mL/100 g body weight/24 hours, Means±SEM (N=8), p=0.010 (Student's t test).The chemically-induced gastric cancer model was established according to our previous report (22). In brief, WT mice and M3KO mice were exposed to N-Methyl-N-nitrosourea (MNU, Sigma Chemicals), which was dissolved in distilled water at a concentration of 240 ppm and freshly prepared twice per week for administration in drinking water in light-shielded bottles ad libitum. Mice at 4 weeks of age were given drinking water containing MNU on alternate weeks for a total of 10 weeks. H pylori infection was induced in H+/K+-ATPase-IL-1βmice (23) by inoculation with pre-mouse Sydney strain 1 [PMSS1]). Three inocula (0.2 mL of Hp, 10¹⁰ colony-forming units/mL) were delivered every other day by oral gavage using a sterile gavage needle.

In the present study, all mice, including INS-GAS mice (Trondheim) and WT mice (FVB and C57BL/6) (Taconic, Denmark) were housed 3-4 mice per cage (either INS-GAS or WT mice) on wood chip bedding with a 12-hour light/dark cycle, room temperature of 22° C. and 40-60% relative humidity. INS-GAS mice and FVB WT mice were housed at the standard housing conditions in a specific pathogen free environment in agreement with FELASA (Federation of European Laboratory Animal Science Association) recommendations. M3KO mice, Hp-infected H⁺/K⁺-ATPase-IL-1β mice and WT controls (C57BL/6 mice) were housed in guaranteed animal facility in Trondheim. All the mice had free access to tap water and standard pellet food (RM1 801002, Scanbur BK AS). Animal experiments were approved by the Norwegian National Animal Research Authority (Forsøksdyrutvalget, FDU) and by the Columbia University Institutional Animal Care and Use Committee (IACUC).

Experimental designs

567 mice were divided into 14 experimental groups (Table 1). In each experiment, mice were randomly divided into different subgroups. The animals, samples and treatments were coded until the data were analyzed.

In the 1^{st} experiment, 107 INS-GAS mice at 6 months of age underwent bilateral truncal vagotomy with pyloroplasty (VTPP) (6 males, 19 females), pyloroplasty alone (PP) (7 males, 18 females), unilateral anterior truncal vagotomy (UVT) (11 males, 19 females) or sham operation (11 males, 16 females). Six months after surgery (at 12 months of age), the animals were euthanized and the anterior and posterior parts of the stomachs were collected for histopathological and immunohistochemical analyses.

In the 2^{nd} experiment, 20 WT mice (FVB, the same genetic background as INS-GAS mice) were exposed to MNU for one week every other week for 5 cycles (10 weeks). At 3.5 months of age, half of the MNU-treated mice underwent VTPP and the other half underwent a sham operation (PP). All the mice were euthanized at 13 months of age, and the stomachs were examined macroscopically and collected for histopathological analysis.

In the 3^{rd} experiment, 24 H⁺/K⁺-ATPase-IL-1β mice (14 males and 10 females, 10 generation backcrossing to C57BL/6J) were inoculated with Hp at 3.5 months of age. At 12 months of age, all the mice underwent UVT and were euthanized 6 months later. The stomachs were examined macroscopically and collected for histopathological analysis.

In the 4^{th} experiment, 16 INS-GAS mice (5 males and 11 females) at 6 months of age underwent unilateral Botox® treatment and were euthanized at 12 months of age. The anterior and posterior parts of the stomachs were collected for histopathological analysis.

In the 5^{th} experiment, 64 INS-GAS mice at 8 months (7 males and 10 females), 10 months (6 males and 8 females) and 12 months (6 males and 6 females) of age underwent UVT, and 21 age-matched mice (8 males and 13 females) had no surgery. At 18 months of age, all surviving mice including 12 (6 males and 6 females) from 8 months group, 9 from 10 months group (3 males and 6 females), 8 from 12 months group (4 males and 4 females) and 10 from un-operated group (4 males and 6 females) were euthanized, and the anterior and posterior parts of the stomachs were collected for histopathological analysis and genome-wide gene expression profiling analysis. Survival analysis was also performed.

In the 6^{th} experiment, 26 INS-GAS mice at 12 months of age underwent Botox® treatments (only anterior or both anterior and posterior sides of stomach with or without UVT) or vehicle injection (both anterior and posterior sides of stomach) and were euthanized at 14 months of age. Both the anterior and posterior parts of the stomachs were collected for histopathological analysis.

In the 7^{th} experiment, 100 INS-GAS mice at 12-14 months of age received no treatment (6 males and 6 females), or treatment with saline (4 males and 6 females) with unilateral Botox® treatment, 5-fluorouracil (5-FU) (4 males and 6 females) with unilateral Botox® treatment, oxaliplatin (6 males and 7 females) with unilateral Botox® treatment, or 5-FU+oxaliplatin with sham operation (6 males and 9 females), unilateral Botox® treatment (11 males and 13 females) or UVT (6 males and 10 females), respectively. Thus, denervation treatment was applied to only half of the stomach, such that the non-denervated half of the stomach in each animal served as a control, either as a chemotherapy only or as an untreated control. All mice were euthanized 2 months after starting the treatments, except for mice that died before the end of study, and both the anterior and posterior parts of the stomachs were collected for histopathological analysis. Survival analysis was also performed.

In the 8^{th} experiment, 16 INS-GAS mice at 6 months of age underwent UVT, and were euthanized at 2 months (1 male and 4 females), 4 months (2 males and 3 females), and 6 months (2 males and 4 females) postoperatively. Then the anterior and posterior parts of the stomachs were collected for genome-wide gene expression profiling.

In the 9^{th} experiment, 44 INS-GAS mice at 12-14 months of age received saline (3 males and 3 females), 5-FU+oxaliplatin (5 males and 8 females), darifenacin (6 males and 6 females) or combination of 5-FU+oxaliplatin and darifenacin (8 males and 5 females), respectively. Two months after starting the treatments, the mice were euthanized, and both the anterior and posterior parts of the stomachs collected for histopathological analysis.

In the 10^{th} experiment, both INS-GAS mice (6 males and 6 females) and wild-type (WT) mice (10 males and 10 females) were subjected either to UVT or no treatment. Six months after surgery (at 12 months of age), the animals were euthanized and the anterior and posterior parts of the stomachs were collected for gene expression analysis.

In the 11^{th} experiment, 10 MNU mice (see the 2^{nd} experiment) (5 males and 5 females) were subjected to PP or VTPP at 6 months of age and euthanized 4 months later. The stomachs were collected for qRT-PCR analysis.

In the 12^{th} experiment, 7 M₃KO mice (4 males and 3 females) and 13 WT mice (5 males and 8 females) (C57BL/6, the same genetic background as M₃ KO mice) were exposed to MNU (same as the 2^{nd} experiment) and euthanized at 11 months of age. The stomachs were examined macroscopically and were collected for histopathological analysis.

In the 13^{th} experiment, 37 mice (20 males, 17 females) at 12-18 months of age underwent a topical application of acetic acid on the anterior side of the stomach with or without simultaneous UVT, and were euthanized 1 week (5 males, 7 females), 2 weeks (3 males, 9 females) or 3 weeks (12 males, 1 female) later, and the anterior part of the stomachs were collected for histopathological analysis.

In the 14^{th} experiment, 10 Lgr5-GFP mice (all males) were treated with MNU at 2 months of age, subjected to PP or VTPP at 19 weeks of age, and were euthanized at 25 weeks of age.

Animal Surgery

All surgical procedures were performed under isoflurane inhalation anesthesia (2-3%), with buprenorphine (0.1 mg/kg subcutaneously) given as postoperative analgesia. The abdominal cavity was accessed through a midline incision. The sham operation consisted of a laparotomy with mild manipulation of organs, including identification of the vagus nerve. PP was done by longitudinal incision of the pyloric sphincter followed by transverse suturing. VTPP was performed by subdiaphragmatic dissection of both the anterior and posterior vagal trunks, and simultaneous PP to prevent post-vagotomy delayed gastric emptying. In UVT only the anterior truncal vagus nerve was cut (FIG. S2), leading to a specific vagal denervation of the anterior aspects of the stomach with preserved pyloric function making PP unnecessary. Sample collection was done under inhalation anesthesia as described, and the animals euthanized by exsanguination while still under anesthesia. Body weight was not affected by VTPP, PP and UVT (FIG. 11).

Botox Treatment

Botox® 100 U (Botox® Allergan Inc.) was dissolved in 0.9% cold saline and 1% methylene blue (to visualize the injection) achieving a concentration of 0.25 U of Botox®/mL. The Botox® solution was injected subserosally along the greater curvature into the anterior (Unilateral Botox® treatment) or both anterior and posterior sides (Bilateral Botox® treatment) of the stomach (only corpus area where tumor developed) at the dose of 0.05 U/mouse (0.2 mL/mouse) or 0.1 U/mouse (0.4 mL/mouse), respectively, once per month until the end of the study. In the control group, the vehicle solution was prepared with 0.9% cold saline and 1% methylene blue and injected to both anterior and posterior sides of the stomach (only to the corpus area where tumor developed) at the volume of 0.4 mL/mouse.

Chemotherapy and M₃ Receptor Antagonist Treatment

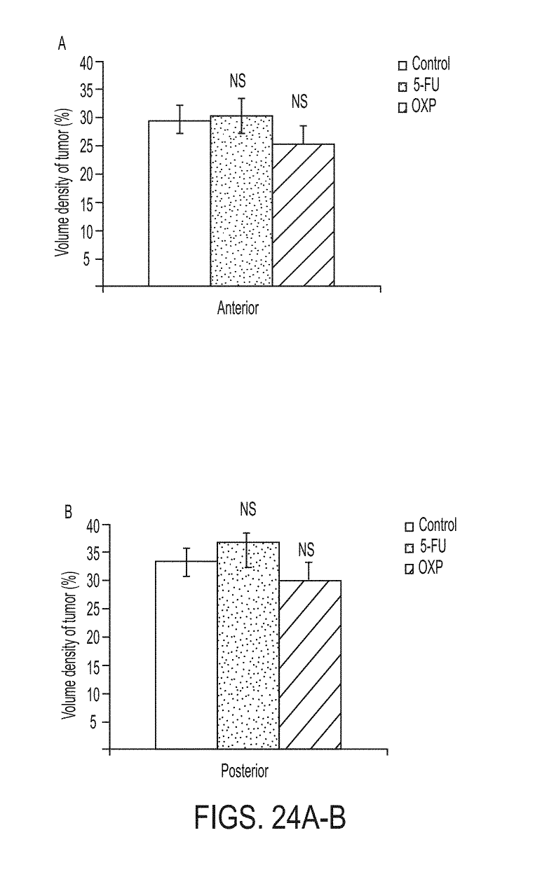

5-Fluorouracil (5-FU, Flurablastin®, Pfizer, Inc.) was diluted in saline and given at a dose of 25 mg/kg in a volume of 1 mL. Oxaliplatin (Hospira, Inc.) was diluted in saline and given at 5 mg/kg in 1 mL. Combination of 5-FU (25 mg/kg in 0.5 mL) and oxaliplatin (5 mg/kg in 0.5 mL) was given at the same time but drugs injected separately. Chemotherapy was given by intraperitoneal injection weekly in 2 cycles, namely 3 injections in the 1^{st} month (one week after the 1^{st} Botox® or UVT surgery), and 3 injections in the 2^{nd} month (starting at one week after the 2^{nd} Botox® or no UVT). Age- and sex-matched mice received intraperitoneal injection of saline (1 mL) as controls. The injection needle was 27 G. The dosages and regimens were made based on our pilot experiments for selecting the doses. There was no effect on tumor size by 5-FU or oxaliplatin alone (FIG. 24).

Darifenacin hydrobromide (Santa Cruz Biotechnology) was given at a dose of 1 mg/kg/h for 2 months via an osmotic mini-pump (ALZET 2006) as reported previously (48).

Tumor Regeneration Model

Topical application of acetic acid was found to promptly cause necrosis in the tumor tissue in INS-GAS mice Under isofluran anesthesia, the stomach was exposed through a midline abdominal incision and 60% acetic acid was topically applied to the serosa of the anterior side of the stomach for 60 seconds using a 5 mm ID cylindrical metal mold. In the experiment of acetic acid-induced necrotic ulcer with UVT, the mice underwent UVT and acetic acid application during the surgery.

Pathological and Immunohistochemical Analyses

The stomachs were removed, opened along the great curvature, washed in 0.9% NaCl and pinned flat on a petri-dish-silicone board. Each stomach was photographed digitally; the tumor profiles in both anterior and posterior sides were drawn separately and subjected to morphometric analysis of the volume density (expressed as the percentage of glandular volume occupied by the tumor) using point-counting technique (multipurpose test system). A test system comprising a 1.0 cm square lattice was placed over each photograph (40×30 cm²), and the numbers of test points overlying the tumor and gastric glandular area were determined. The samples for histology comprised multiple linear strips along the greater curvature of the stomach wall, extending from the squamocolumnar junction through the antrum. Samples were fixed in 4% formaldehyde for 8-12 hours at room temperature and embedded in paraffin. Sections (4 μm thick) were stained with hematoxylin and eosin. Pathological evaluation was performed by one comparative pathologist (S.M.) and one histologist (C-M.Z.) who were blinded to the sample source. The gastric lesions were scored on an ascending scale from 0 to 4 using criteria adopted from previous reports (21). Inflammation scoring was done according to patchy infiltration of mixed leukocytes in mucosa and/or submucosa (N^{o}. 1), multifocal-to-coalescing leukocyte infiltration not extended below submucosa (N^{o}. 2), marked increased in leukocytes with lymphoid follicles +/− extension into tunica muscularis (N^{o}. 3), and effacing transmural inflammation (N^{o}. 4). The epithelial defects were defined as gland dilatation, surface erosions and gland atrophy, and ulceration and fibrosis. Immunohistochemistry was performed using a DAKO AutoStainer (Universal Staining System with DAKO EnVision System, Dako). Antibodies used were Ki67 (1:100; code M7249, Dako), PCNA (1:100, code M0879, Dako), CD44 (1:100, code 550538, BD Pharmingen), CD44V6 (1:200, code AB2080, Millipore), PGP9.5 (1:1000, code Z5116, Dako, and code 7863-0504, AbD Serotec), peripherin (1:500, code AB1530, Millipore), β-catenin (1:500, code 610654, BD Transduction Laboratories), Alexa Fluor® 488 Phalloidin (1:200, code A12379, Life Technologies), Alexa Fluor® 555 Goat Anti-Rabbit IgG (H+L)(1:200, code A21428, Life Technologies). Cell proliferation rate is expressed as number of Ki67 or PCNA immunoreactive cells/gland. There was no difference between the two markers between two labs (TW and DC). Slides were visualized on a Nikon TE2000-U and representative microphotos were taken. Positive-stained cells with nuclei were counted only in dysplastic glands and at least 50 glands were counted per animal in a blinded fashion, and results expressed as numerical densities (number of cells per gland, number of cells per 10 glands or per object field). Positive-stained nerves were quantitated by ImageJ software and expressed as positive area per total mucosal area.

Vagus Nerve Fibers and Terminals in the Mouse Stomach Traced with Carbocyanine Dye (DiI)

The esophagus, diaphragm and stomach were removed from adult mice and fixed for 3 days with formaldehyde. DiI crystals were placed on the anterior and posterior thoracic vagal trunks about 1 cm above the diaphragm, which was left undisturbed. The preparation was incubated at 37° C. in PBS containing 0.5% sodium azide in a sealed container for 3 months. Following incubation, the stomach was opened along the greater curvature, the mucosa and submucosa were removed and the preparations were mounted, serosal side up, in buffered glycerol for microscopic examination. DiI fluorescence was viewed with a Leica CTR6000 microscope equipped with a cooled CCD camera and computer assisted video imaging. The entire gastric wall was scanned with a 2.5×objective and a montage was made of the resulting images. In order to observe the density of DiI-labeled vagal fibers within the myenteric plexus, additional images were obtained at higher magnification in the lesser curvature close to the esophagogastric junction, and the greater curvature. The density of DiI-labeled fibers was estimated by point-counting technique. A test system comprising a 1.0 cm square lattice was placed over each photograph, and the numbers of test points overlying the DiI-labeled fibers and the visual field were determined

RNA Isolation, Gene Expression Profiling by Microarray, qRT-PCR Arrays and qRT-PCR in Mice and Humans

The collected mouse and human stomach samples were kept frozen at −80° C. until further processing. Total RNA from the frozen stomach samples was isolated and purified using an Ultra-Turrax rotating-knife homogenizer and the Miramar miRNA Isolation Kit (AM1560, Ambion) according to the manufacturer's instructions. Concentration and purity of total RNA were assessed using a NanoDrop (NanoDrop Technologies, Inc.) photometer. The A260/280 ratios were 2.05±0.01 for mouse samples and 1.96±0.10 for human samples (mean±SEM). RNA integrity was assessed using a Bioanalyzer (Agilent Technologies) and found satisfactory with RNA integrity number (RIN) values 9.1±0.1 for mouse samples, and 8.7±0.9 for human samples (means±SEM). The microarray gene expression analysis followed standard protocols, analyzing 300 ng total RNA per sample with the Illumina MouseWG-6 and HumanHT-12 Expression BeadChips (Illumina). Microarray data were confirmed by qRT-PCR array (RT² Profiler PCR Array, SABiosciences) (StepOnePlus™, Applied Biosystems). Mouse microarray data were deposited in the Gene Expression Omnibus (GEO accession no. GSE30295), and human data in ArrayExpress (accession no. E-MTAB-1338).

Longitudinal strips of gastric tissue from the anterior wall as well as the posterior wall were harvested and snap-frozen in dry ice and kept in −80° C. freezer until processed for analysis. Total RNA was extracted with Nucleospin RNA II kit (Clontech) and cDNA was synthesized by Superscript III First-strand Synthesis System for RT-PCR (Invitrogen)(for primer sequences used in this experiment, see Supplementary Table 3). Expression levels of indicated genes were quantified by real-time PCR assays using SYBR Green dye and the Applied Biosystems 7300 Real Time PCR System.

Fluorescence-Activated Cell Sorting (FACS)

Single epithelial cells were isolated from Lgr5-GFP mouse stomachs. Isolated crypts were dissociated with TrypLE Express (Invitrogen) including 1 mg/ml DNase I (Roche Applied Science) for 10 minutes at 37° C. Dissociated cells were passed through a 20-μm cell strainer, washed with 2% FBS/PBS, and sorted by FACS (BD FACSAria Cell Sorter III). Viable single epithelial cells were gated by forward scatter, side scatter and a pulse-width parameter, and negative staining for propidium iodide. Cells expressing high and low levels of GFP, and GFP-negative cells were sorted separately, and RNA was isolated by using RNAqueous®-Micro Kit (Ambion).

In vitro Culture System

Wild-type (WT), Ubiquitin C-green fluorescent protein (UBC-GFP), or Gt(ROSA)26Sortm4(ACTB-tdTomato,-EGFP)Luo/J mice (The Jackson laboratory) were used for in vitro culture. Gastric gland isolation and culture were performed, as described previously (9) with minor modifications. Removed stomachs from WT and M₃KO mice were opened longitudinally, chopped into approximately 5 mm pieces, and incubated in 8 mM EDTA in PBS for 60 minutes on ice. The tissue fragments were suspended vigorously, yielding supernatants enriched in gastric glands. Gland fractions were centrifuged at 900 rpm for 5 minutes at 4° C. and diluted with advanced DMEM/F12 (Invitrogen) containing B27, N2, 1 μM n-Acetylcysteine, 10 mM HEPES, penicillin/streptomycin, and Glutamax (all Invitrogen). Glands were embedded in extracellular matrix (Fisher Bioservices/NCI Frederick Central Repository) and 400 crypts/well were seeded on pre-warmed plate. Advanced DMEM/F12 medium containing 50 ng/mL EGF, 100 ng/mL Noggin, and 1 μg/mL R-spondin1 was applied. Wnt3a (PeproTech, Rocky Hill, N.J.) was added at 100 ng/mL when indicated. Growth factors were added every other day and the entire medium was changed twice a week. Passage was performed at day 7 as described previously (9). Mouse primary neuronal cells were prepared following the protocol described previously (33). Neuronal cells were mixed with extracted gastric crypts in the extracellular matrix at the ratio of crypt:neuron 1:5. The enteric nervous system was isolated from guinea pigs as described previously (34). Botox®, scopolamine hydrochloride, and pilocarpine hydrochloride (Sigma) were dissolved in PBS and added in the cultured medium every other day. The images of gastric organoids were acquired using fluorescent microscopy (Nikon, TE2000-U) and two-photon microscopy (Nikon, A1RMP). Isolation of mRNA from cultured organoids was performed by using NucleoSpin RNA XS kit (Clontech Laboratories Inc) according to manufacturer's instructions. The first-strand complementary DNA was synthesized using the ImProm-II Reverse Transcription System (Promega). Amplification was performed using the ABI PRISM 7300 Quantitative PCR System (Applied Biosystems). Use of the animals involved in this experiment was approved by the Columbia University Institutional Animal Care and Use Committee.

Patients and Methods

Three cohort studies were included (Table 2). In the 1^{st} study, human stomach specimens (both tumors and the adjacent non-tumor tissues) were taken immediately after gastrectomy from 17 patients during 2005 to 2010 at St. Olav's University Hospital, Trondheim, Norway for gene expression profiling analysis. All patients were diagnosed histologically as primary gastric carcinoma of stage I-IV. 10 of 17 patients were H pylori positive at the time of surgery. In the 2^{nd} study, human stomach tissues were obtained from 120 gastric cancer patients who underwent curative surgical resection from 2001 to 2008 at Gifu University Hospital, Gifu, Japan. All patients were diagnosed histologically as primary gastric carcinoma of stage II, III or IV. Immunohistochemical analysis of the nerve density was performed with PGP9.5 antibody. Low and high expression of PGP9.5 were defined with respect to the median of the volume density of PGP9.5. In the 3^{rd} study, clinical data of 37 patients with gastric stump cancer (GSC) who had received distal gastrectomy with or without vagotomy during 1962 to 1995 at the National Cancer Center Hospital East, Chiba, Japan were evaluated. GSC was defined as gastric cancer that occurred ≧5 years (from 5 to 36 years) after curative distal gastrectomy regardless of the original benign or malignant disease. GSC included in this study was adenocarcinoma infiltrating the mucosal or submucosal layer. The tumor location was recorded according to the recommendation by the Japanese Gastric Cancer Association: anterior or posterior wall, or lesser or greater curvature. All the study protocols were approved by the ethics committees in Japan and Norway and written informed consent was obtained from patients.

Data Analysis

Values were expressed as means±SEM. Pairwise comparisons between experimental groups and between anterior and posterior sides of the stomach were performed with the paired and unpaired t-test as appropriate. All tests were two sided with a signfiance cutoff of 0.05. Comparisons between more than 2 groups were performed by ANOVA, followed by Dunnett's test ^{A1} or Turkey's test ^{A2, A3} as appropriate. Comparisons with categorical independent variables were performed using Fisher's exact test ^{A4, A5} . Kaplan-Meier survival curves ^{A6} were calculated and were analyzed by the Cox proportional hazard method ^{A7}. Tumor incidence was analyzed by Fisher's exact test. Affymetrix microarray data was normalized using RMA ^{A8} and Illumina microarray data was analyzed using Lumi ^{A9}. Both qRT-PCR and microarray data were analyzed on the log₂ scale. The significance of differential expression of qRT-PCR data was analyzed using parametric frequentist statistics and microarray data was analyzed using the empirical Bayesian method implemented in Limma A¹⁰. Gene expression profiles from both microarray and qRT-PCR were analyzed independently by a paired robust t-test for mouse samples or a paired t-test for human samples. Paired t-statistics were computed by fitting a linear robust ^{A11} or non-robust regression to the anterior and posterior stomach samples within each mouse or to the cancer and the adjacent non-cancerous tissue samples within each patient. For microarray data, transcripts with Benjamini-Hochberg false discovery rates ^{A12} less than 0.05 were considered to be differentially expressed. Regulated KEGG pathways (Kyoto Encyclopedia of Genes and Genomes) ^{A13} were identified using Signaling Pathway Impact Analysis ^{A14, A15} . All of the above calculations were performed in the R ^{A16, A17} /Bioconductor ^{A18} software environment.

### REFERENCES

1. Y. Katayama, M. Battista, W. M. Kao, A. Hidalgo, A. J. Peired, S. A. Thomas, P. S. Frenette, Signals from the sympathetic nervous system regulate hematopoietic stem cell egress from bone marrow. Cell 124, 407-421 (2006).

2. O. Lundgren, M. Jodal, M. Jansson, A. T. Ryberg, L. Svensson, Intestinal epithelial stem/progenitor cells are controlled by mucosal afferent nerves. PLOS One 6, e16295 (2011).

3. R. R. Mattingly, A. Sorisky, M. R. Brann, I. G. Macara, Muscarinic receptors transform NIH 3T3 cells through a Ras-dependent signaling pathway inhibited by the Ras-GTPase-activating protein SH3 domain. Mol. Cell. Biol. 14, 7943-7952 (1994).

4. G. E. Ayala, H. Dai, M. Powell, R. Li, Y. Ding, T. M. Wheeler, D. Shine, D. Kadmon, T. Thompson, B. J. Miles, M. M. Ittmann, D. Rowley, Cancer-related axonogenesis and neurogenesis in prostate cancer. Clin. Cancer Res. 14, 7593-7603 (2008).

5. N. Shah, S. Khurana, K. Cheng, J. P. Raufman, Muscarinic receptors and ligands in cancer. Am. J. Physiol. Cell Physiol. 296, C221-C232 (2009).

6. J. P. Raufman, J. Shant, G. Xie, K. Cheng, X. M. Gao, B. Shiu, N. Shah, C. B. Drachenberg, J. Heath, J. Wess, S. Khurana, Muscarinic receptor subtype-3 gene ablation and scopolamine butylbromide treatment attenuate small intestinal neoplasia in Apcmin/+ mice. Carcinogenesis 32, 1396-1402 (2011).

7. C. Magnon, S. J. Hall, J. Lin, X. Xue, L. Gerber, S. J. Freedland, P. S. Frenette, Autonomic nerve development contributes to prostate cancer progression. Science 341, 1236361 (2013).

8. R. Pardal, M. F. Clarke, S. J. Morrison, Applying the principles of stem-cell biology to cancer. Nat. Rev. Cancer 3, 895-902 (2003).

9. N. Barker, M. Huch, P. Kujala, M. van deWetering, H. J. Snippert, J. H. van Es, T. Sato, D. E. Stange, H. Begthel, M. van den Born, E. Danenberg, S. van den Brink, J. Korving, A. Abo, P. J. Peters, N. Wright, R. Poulsom, H. Clevers, Lgr5+ve stem cells drive self-renewal in the stomach and build long-lived gastric units in vitro. Cell Stem Cell 6, 25-36 (2010).

10. J. Ferlay, H. R. Shin, F. Bray, D. Forman, C. Mathers, D. M. Parkin, Estimates of worldwide burden of cancer in 2008: GLOBOCAN 2008. Int. J. Cancer 127, 2893-2917 (2010).

11. A. Jemal, M. M. Center, C. DeSantis, E. M. Ward, Global patterns of cancer incidence and mortality rates and trends. Cancer Epidemiol. Biomarkers Prey. 19,1893-1907 (2010).

12. R. Håkanson, S. Vallgren, M. Ekelund, J. F. Rehfeld, F. Sundler, The vagus exerts trophic control of the stomach in the rat. Gastroenterology 86,28-32 (1984).

13. J. Axelson, M. Ekelund, R. Håkanson, F. Sundler, Gastrin and the vagus interact in the trophic control of the rat oxyntic mucosa. Regul. Pept. 22,237-243 (1988).

14. G. Lundegårdh, A. Ekbom, J. K. McLaughlin, O. Nyrén, Gastric cancer risk after vagotomy. Gut 35,946-949 (1994).

15. S. Bahmanyar, W. Ye, P. W. Dickman, O. Nyrén, Long-term risk of gastric cancer by subsite in operated and unoperated patients hospitalized for peptic ulcer. Am. J. Gastroenterol. 102, 1185-1191 (2007).

16. P. Correa, C. Cuello, E. Duque, Carcinoma and intestinal metaplasia of the stomach in Colombian migrants. J. Natl. Cancer Inst. 44,297-306 (1970).

17. M. Cassaro, M. Rugge, O. Gutierrez, G. Leandro, D. Y. Graham, R. M. Genta, Topographic patterns of intestinal metaplasia and gastric cancer. Am. J. Gastroenterol. 95,1431-1438 (2000).

18. T. C. Wang, C. A. Dangler, D. Chen, J. R. Goldenring, T. Koh, R. Raychowdhury, R. J. Coffey, S. Ito, A. Varro, G. J. Dockray, J. G. Fox, Synergistic interaction between hypergastrinemia and Helicobacter infection in a mouse model of gastric cancer. Gastroenterology 118,36-47 (2000).

19. J. G. Fox, T. C. Wang, Inflammation, atrophy, and gastric cancer. J. Clin. Invest. 117, 60-69 (2007).

20. P. Ericsson, R. Håkanson, J. F. Rehfeld, P. Norlén, Gastrin release: Antrum microdialysis reveals a complex neural control. Regul. Pept. 161,22-32 (2010). 21. A. B. Rogers, N. S. Taylor, M. T. Whary, E. D. Stefanich, T. C. Wang, J. G. Fox, Helicobacter pylori but not high salt induces gastric intraepithelial neoplasia in B6129 mice. Cancer Res. 65,10709-10715 (2005).

22. H. Tomita, S. Takaishi, T. R. Menheniott, X. Yang, W. Shibata, G. Jin, K. S. Betz, K. Kawakami, T. Minamoto, C. Tomasetto, M. C. Rio, N. Lerkowit, A. Varro, A. S. Giraud, T. C. Wang, Inhibition of gastric carcinogenesis by the hormone gastrin is mediated by suppression of TFF1 epigenetic silencing. Gastroenterology 140,879-891 (2011).

23. S. Tu, G. Bhagat, G. Cui, S. Takaishi, E. A. Kurt-Jones, B. Rickman, K. S. Betz, M. Penz-Oesterreicher, O. Bjorkdahl, J. G. Fox, T. C. Wang, Overexpression of interleukin-1b induces gastric inflammation and cancer and mobilizes myeloid-derived suppressor cells in mice. Cancer Cell 14,408-419 (2008).

24. D. Dressler, F. Adib Saberi, Botulinum toxin: Mechanisms of action. Eur. Neurol. 53, 3-9 (2005).

25. P. Polakis, Drugging Wnt signaling in cancer. EMBO J. 31,2737-2746 (2012).

26. F. Takahashi-Yanaga, M. Kahn, Targeting Wnt signaling: Can we safely eradicate cancer stem cells? Clin. Cancer Res. 16,3153-3162 (2010).

27. M. Zöller, CD44: Can a cancer-initiating cell profit from an abundantly expressed molecule? Nat. Rev. Cancer 11,254-267 (2011).

28. S. Takaishi, T. Okumura, S. Tu, S. S. Wang, W. Shibata, R. Vigneshwaran, S. A. Gordon, Y. Shimada, T. C. Wang, Identification of gastric cancer stem cells using the cell surface marker CD44. Stem Cells 27,1006-1020 (2009).

29. J. Schuijers, H. Clevers, Adult mammalian stem cells: The role of Wnt, Lgr5 and R-spondins. EMBO J. 31,2685-2696 (2012).

30. J. Chen, Y. Li, T. S. Yu, R. M. McKay, D. K. Burns, S. G. Kernie, L. F. Parada, A restricted cell population propagates glioblastoma growth after chemotherapy. Nature 488,522-526 (2012).

31. S. Okabe, Y. Kodama, H. Cao, H. Johannessen, C. M. Zhao, T. C. Wang, R. Takahashi, D. Chen, Topical application of acetic acid in cytoreduction of gastric cancer. A technical report using mouse model. J. Gastroenterol. Hepatol. 27 (Suppl. 3), 40-48 (2012).

32. P. Song, H. S. Sekhon, X. W. Fu, M. Maier, Y. Jia, J. Duan, B. J. Proskosil, C. Gravett, J. Lindstrom, G. P. Mark, S. Saha, E. R. Spindel, Activated cholinergic signaling provides a target in squamous cell lung carcinoma. Cancer Res. 68,4693-4700 (2008).

33. C. B. Westphalen, S. Asfaha, Y. Hayakawa, Y. Takemoto, D. J. Lukin, A. H. Nuber, A. Brandtner, W. Setlik, H. Remotti, A. Muley, X. Chen, R. May, C. W. Houchen, J. G. Fox, M. D. Gershon, M. Quante, T. C. Wang, Long-lived intestinal tuft cells serve as colon cancer-initiating cells. J. Clin. Invest. 124, 1283-1295 (2014).

34. A. A. Gershon, J. Chen, M. D. Gershon, A model of lytic, latent, and reactivating varicella-zoster virus infections in isolated enteric neurons. J. Infect. Dis. 197 (Suppl. 2), S61-S65 (2008).

35. N. Barker, H. Clevers, Leucine-rich repeat-containing G-protein-coupled receptors as markers of adult stem cells. Gastroenterology 138, 1681-1696 (2010).

36. M. Tatsuta, H. Yamamura, H. Iishi, M. Ichii, S. Noguchi, M. Baba, H. Taniguchi, Promotion by vagotomy of gastric carcinogenesis induced by N-methyl-N′-nitro-N-nitrosoguanidine in Wistar rats. Cancer Res. 45, 194-197 (1985).

37. M. Tatsuta, H. Iishi, H. Yamamura, M. Baba, H. Taniguchi, Effects of bilateral and unilateral vagotomy on gastric carcinogenesis induced by N-methyl-N′-nitro-N-nitrosoguanidine in Wistar rats. Int. J. Cancer 42, 414-418 (1988).

38. P. S. Frenette, S. Pinho, D. Lucas, C. Scheiermann, Mesenchymal stem cell: Keystone of the hematopoietic stem cell niche and a stepping-stone for regenerative medicine. Annu. Rev. Immunol. 31, 285-316 (2013).

39. E. R. Gross, M. D. Gershon, K. G. Margolis, Z. V. Gertsberg, R. A. Cowles, Neuronal serotonin regulates growth of the intestinal mucosa in mice. Gastroenterology 143, 408-417.e2 (2012).

40. T. Sato, J. H. van Es, H. J. Snippert, D. E. Stange, R. G. Vries, M. van den Born, N. Barker,N. F. Shroyer, M. van de Wetering, H. Clevers, Paneth cells constitute the niche for Lgr5 stem cells in intestinal crypts. Nature 469, 415-418 (2011).

41. D. W. Powell, I. V. Pinchuk, J. I. Saada, X. Chen, R. C. Mifflin, Mesenchymal cells of the intestinal lamina propria. Annu. Rev. Physiol. 73, 213-237 (2011).

42. H. F. Farin, J. H. Van Es, H. Clevers, Redundant sources of Wnt regulate intestinal stem cells and promote formation of Paneth cells. Gastroenterology 143, 1518-1529.e7 (2012).

43. D. Revesz, M. Tjernstrom, E. Ben-Menachem, T. Thorlin, Effects of vagus nerve stimulation on rat hippocampal progenitor proliferation. Exp. Neurol. 214,259-265 (2008).

44. D. Cassiman, L. Libbrecht, N. Sinelli, V. Desmet, C. Denef, T. Roskams, The vagal nerve stimulates activation of the hepatic progenitor cell compartment via muscarinic acetylcholine receptor type 3. Am. J. Pathol. 161,521-530 (2002).

45. T. Aihara, T. Fujishita, K. Kanatani, K. Furutani, E. Nakamura, M. M. Taketo, M. Matsui, D. Chen, S. Okabe, Impaired gastric secretion and lack of trophic responses to hypergastrinemia in M3 muscarinic receptor knockout mice. Gastroenterology 125,1774-1784 (2003).

46. S. Salmanian, S. M. Najafi, M. Rafipour, M. R. Arjomand, H. Shahheydari, S. Ansari, L. Kashkooli, S. J. Rasouli, M. S. Jazi, T. Minaei, Regulation of GSK-3b and b-catenin by Gaq in HEK293T cells. Biochem. Biophys. Res. Commun. 395,577-582 (2010).

47. E. R. Spindel, Muscarinic receptor agonists and antagonists: Effects on cancer. Handb. Exp. Pharmacol. 451-468 (2012).

48. J. P. Raufman, R. Samimi, N. Shah, S. Khurana, J. Shant, C. Drachenberg, G. Xie, J. Wess, K. Cheng, Genetic ablation of M3 muscarinic receptors attenuates murine colon epithelial cell proliferation and neoplasia. Cancer Res. 68,3573-3578 (2008).

49. P. W. Veenboer, J. L. Bosch, Long-term adherence to antimuscarinic therapy in everyday practice: A systematic review. J. Urol. 191,1003-1008 (2014).

50. P. Song, H. S. Sekhon, A. Lu, J. Arredondo, D. Sauer, C. Gravett, G. P. Mark, S. A. Grando, E. R. Spindel, M3 muscarinic receptor antagonists inhibit small cell lung carcinoma growth and mitogenactivated protein kinase phosphorylation induced by acetylcholine secretion. Cancer Res. 67,3936-3944 (2007).

51. H. Oshima, M. Oshima, Mouse models of gastric tumors: Wnt activation and PGE2 induction. Pathol. Int. 60,599-607 (2010).

52. J. H. van Es, P. Jay, A. Gregorieff, M. E. van Gijn, S. Jonkheer, P. Hatzis, A. Thiele, M. van den Born, H. Begthel, T. Brabletz, M. M. Taketo, H. Clevers, Wnt signaling induces maturation of Paneth cells in intestinal crypts. Nat. Cell Biol. 7,381-386 (2005).

53. S. Fre, S. K. Pallavi, M. Huyghe, M. Laé, K. P. Janssen, S. Robine, S. Artavanis-Tsakonas, D. Louvard, Notch and Wnt signals cooperatively control cell proliferation and tumorigenesis in the intestine. Proc. Natl. Acad. Sci. U.S.A. 106, 6309-6314 (2009).

54. L. Lehtiö, N. W. Chi, S. Krauss, Tankyrases as drug targets. FEBS J. 280, 3576-3593 (2013).

A1. Dunnett C W. A multiple comparison procedure for comparing several treatments with a control. J. Am. Stat. Assoc. 1955; 50:1096-1121.

A2. Tukey J W. The Problem of Multiple Comparisons. unpublished manuscript (reprinted in, The Collected Works of John Tukey, Volume 8, 1994 H. I. Braun, Ed., Chapman and Hall, New York), 1953.

A3. Kramer C W. Extension of multiple range tests to group means with unequal numbers of replications. Biometrics 1956; 12:307-310.

A4. Fisher R A. Statistical Methods for Research Workers. Edinburgh: Oliver and Boyd, 1934.

A5. Yates F. Contingency tables involving small numbers and the x² test. J. Roy. Stat. Soc. Supp. 1934; 1:217-235.

A6. Kaplan E, Meier P. Nonparametric estimation from incomplete observations. J. Am. Stat. Assoc. 1958; 53:457-481.

A7. Cox DR. Regression Models and Life Tables (with discussion). J. Roy. Stat. Soc. Ser. B 1972; 34:187-220.

A8. Irizarry R A, Hobbs B, Collin F, et al. Exploration, normalization, and summaries of high density oligonucleotide array probe level data. Biostatistics 2003; 4:249-64.

A9. Du P, Kibbe W A, Lin S M. lumi: a pipeline for processing Illumina microarray. Bioinformatics 2008; 24:1547-8.

A10. Smyth G K. Linear Models and Empirical Bayes Methods for Assessing Differential Expression in Microarray Experiments. Statistical Applications in Genetics and Molecular Biology 2004; 3:Article 3, http://www.bepress.com/sagmb/vol3/iss ¹/art3/.

A11. Smyth G, Ritchie M, Silver J, et al. Reference Manual to Package Limma: Linear Model for Microarrays, 2014.

Al12. Benjamini Y, Hochberg Y. Controlling the false discovery rate; A practical and powerful approach to multiple testing. J. Roy. Stat. Soc. Ser. B 1995; 57:289-300.

A13. Kanehisa M, Goto S, Kawashima S, et al. The KEGG resource for deciphering the genome. Nucleic Acids Res 2004; 32:D277-80.

A14. Draghici S, Khatri P, Tarca A L, et al. A systems biology approach for pathway level analysis. Genome Res 2007; 17:1537-45.

A15. Tarca A L, Draghici S, Khatri P, et al. A novel signaling pathway impact analysis. Bioinformatics 2009; 25:75-82.

A16. Ihaka R, Gentleman R. R: A language for data analysis and graphics. Journal of Computational and Graphical Statistics 1996; 5:299-314.

A17. Team RC. R: A Language and Environment for Statistical Computing. 3.0.2 ed: R Foundation for Statistical Computing, 2013.

A18. Gentleman R C, Carey V J, Bates D M, et al. Bioconductor: open software development for computational biology and bioinformatics. Genome Biol 2004; 5:R80.

TABLE 1
Animal experimental groups.

| | | | | Age* at |
|---|---|---|---|---|
| | | | Age* at operation | examination |
| | Mouse (N) | Group (N) | (months) | (months) |
| 1 | INS-GAS | Sham (27) | 6 | 12 |
| | (107) | | | |
| | | PP (25) | 6 | 12 |
| | | UVT (30) | 6 | 12 |
| | | VTPP (25) | 6 | 12 |
| 2 | MNU | MNU + PP(11) | | 13 |
| | (FVB)(20) | | | |
| | | MNU + VTPP (9) | 3.5 | 13 |
| 3 | H.p.-infection | Sham (12) | 12 | 18 |
| | (24) | UVT (12) | 12 | 18 |
| 4 | INS-GAS (16) | UB (16) | 6 | 12 |
| 5 | INS-GAS (64) | No surgery (21) | | 18 |
| | | UVT (17) | 8 | 18 |
| | | UVT (14) | 10 | 18 |
| | | UVT (12) | 12 | 18 |
| 6 | INS-GAS (26) | Vehicle (6) | 12 | 14 |
| | | UB (6) | 12 | 14 |
| | | BB (7) | 12 | 14 |
| | | BB + UVT (7) | 12 | 14 |
| 7 | INS-GAS | No treatment (12) | 12-14 | 14-16 |
| | (133) | Saline (10) | 12-14 | 14-16 |
| | | 5-FU (10) | 12-14 | 14-16 |
| | | OXP (13) | 12-14 | 14-16 |
| | | UB + Saline (10) | 12-14 | 14-16 |
| | | UB + 5-FU (10) | 12-14 | 14-16 |
| | | UB + OXP (13) | 12-14 | 14-16 |
| | | Sham + 5-FU + OXP (15) | 12-14 | 14-16 |
| | | UB + 5-FU + OXP (24) | 12-14 | 14-16 |
| | | UVT + 5-FU + OXP (16) | 12-14 | 14-16 |
| 8 | INS-GAS (16) | UVT (5) | 6 | 8 |
| | | UVT (5) | 6 | 10 |
| | | UVT (6) | 6 | 12 |
| 9 | INS-GAS (64) | Saline (19) | 12-14 | 14-16 |
| | | 5-FU + OXP (12) | 12-14 | 14-16 |
| | | Darifenacin (15) | 12-14 | 14-16 |
| | | 5-FU + OXP + darifenacin | 12-14 | 14-16 |
| | | (8) | | |
| 10 | INS-GAS (12) | INS-GAS - No treatment | 6 | 12 |
| | | (6) | | |
| | FVB (20) | INS-GAS - UVT (6) | 6 | 12 |
| | | WT-No treatment (10) | 6 | 12 |
| | | WT-UVT (10) | 6 | 12 |
| 11 | MNU (12) | PP (6) | 6 | 10 |
| | | VTPP (6) | 6 | 10 |
| 12 | M3KO (7) | M3KO + MNU (7) | | 11 |
| | C57BL/6 (13) | WT + MNU (13) | | 11 |
| 13 | INS-GAS (37) | Regeneration** 1 week | 12-18 | 12-18 + 1 week |
| | | (6) | | |
| | | Regeneration 1 week after | 12-18 | 12-18 + 1 week |
| | | UVT (6) | | |
| | | Regeneration 2 weeks (6) | 12-18 | 12-18 + 2 weeks |
| | | Regeneration 2 weeks | 12-18 | 12-18 + 2 weeks |
| | | after UVT (6) | | |
| | | Regeneration 3 weeks (8) | 12-18 | 12-18 + 3 weeks |
| | | Regeneration 3 weeks | 12-18 | 12-18 + 3 weeks |
| | | after UVT (5) | | |
| 14 | Lgr5-GFP (10) | MNU + PP (5) | 4.75 | 6.25 |
| | | MNU + VTPP (5) | 4.75 | 6.25 |

PP: Pyloroplasty
UVT: Unilateral vagotomy
VTPP: Bilateral vagotomy + pyloroplasty
UB: Unilateral botox injection
BB: Bilateral botox injection
OXP: Oxaliplatin
Note:
*preneoplasia at 6 months and neoplasia at 12 months of age in INS-GAS mouse.
**Tumor regeneration was induced by topical application of acetic acid.

TABLE 2
Cohorts of gastric cancer patients.

| | 1^{st} cohort | 2^{nd} cohort | 3^{rd} cohort |
|---|---|---|---|
| Country | Norway | Japan | Japan |
| Purpose | Gene expression | Innervation and | Stump cancer |
| | profiling | tumorigenesis | after vagotomy |
| Study period | 2005-2010 | 2001-2008 | 1962-1998 |
| Patient | | | |
| Number | 17 (14:3) | 120 (78:42) | 37 (31:6) |
| (male:female) | | | |
| Age (y.o.) | 49-86 | 31-92 | 69-90 |
| Pathological stage | I-IV | II-IV | I-II |
| H. pylori status | 10/17 positive | n.d. | n.d. |

TABLE 3
List of qRT-PCR primer used in this study.

| Gene | Forward (5′->3′) | Reverse (5′->3′) |
|---|---|---|
| Lgr5 | TCCAACCTCAGCGTCTTC | TGGGAATGTGTGTCAAAG |
| | (SEQ ID NO: 1) | (SEQ ID NO: 2) |
| Cd44 | CACATATTGCTTCAATGCCT | CCATCACGGTTGACAATAGT |
| | CAG | TATG |
| | (SEQ ID NO: 3) | (SEQ ID NO: 4) |
| Axin2 | ACTGACCGACGATTCCATGT | TGCATCTCTCTCTGGAGCTG |
| | (SEQ ID NO: 5) | (SEQ ID NO: 6) |
| Myc | AGAGCTCCTCGAGCTGTTTG | TGAAGTTCACGTTGAGGGG |
| | (SEQ ID NO: 7) | (SEQ ID NO: 8) |
| Cyclin | TCCTCTCCAAAATGCCAGAG | GGGTGGGTTGGAAATGAAC |
| D1 | (SEQ ID NO: 9) | (SEQ ID NO: 10) |
| Sox9 | AGGAAGCTGGCAGACCAGTA | TCCACGAAGGGTCTCTTCTC |
| | (SEQ ID NO: 11) | (SEQ ID NO: 12) |
| Chrm1 | CAGAAGTGGTGATCAAGATG | GAGCTTTTGGGAGGCTGCTT |
| | CCTAT | (SEQ ID NO: 14) |
| | (SEQ ID NO: 13) | |
| Chrm2 | TGGAGCACAACAAGATCCAG | CCCCTGAACGCAGTTTTCA |
| | AAT | (SEQ ID NO: 16) |
| | (SEQ ID NO: 15) | |
| Chrm3 | CCAGTTGGTGTGTTCTTCCT | AGGAAGAGCTGATGTTGGGA |
| | T | (SEQ ID NO: 18) |
| | (SEQ ID NO: 17) | |
| Chrm4 | GTGACTGCCATCGAGATCGT | CAAACTTTCGGGCCACATTG |
| | AC | (SEQ ID NO: 20) |
| | (SEQ ID NO: 19) | |
| Chrm5 | GGCCCAGAGAGAACGGAAC | TTCCCGTTGTTGAGGTGCTT |
| | (SEQ ID NO: 21) | (SEQ ID NO: 22) |
| Gapdh | TCATTGTCATACCAGGAAAT | AGAAACCTGCCAAGTATGAT |
| | GAG | GAC |
| | (SEQ ID NO: 23) | (SEQ ID NO: 24) |

### EXAMPLE 2

### NGF Regulates Gastric Cancer Development Through Tumor-Associated Neurogenesis Originating from Dclk1+ Neural Progenitors

R26-LSL-NGF mice show increased nerves and spontaneous stomach dysplasia that is blocked when the Muscarinic 3 receptor is knocked out in the gastric epithelium. A similar effect was seen in the colon.

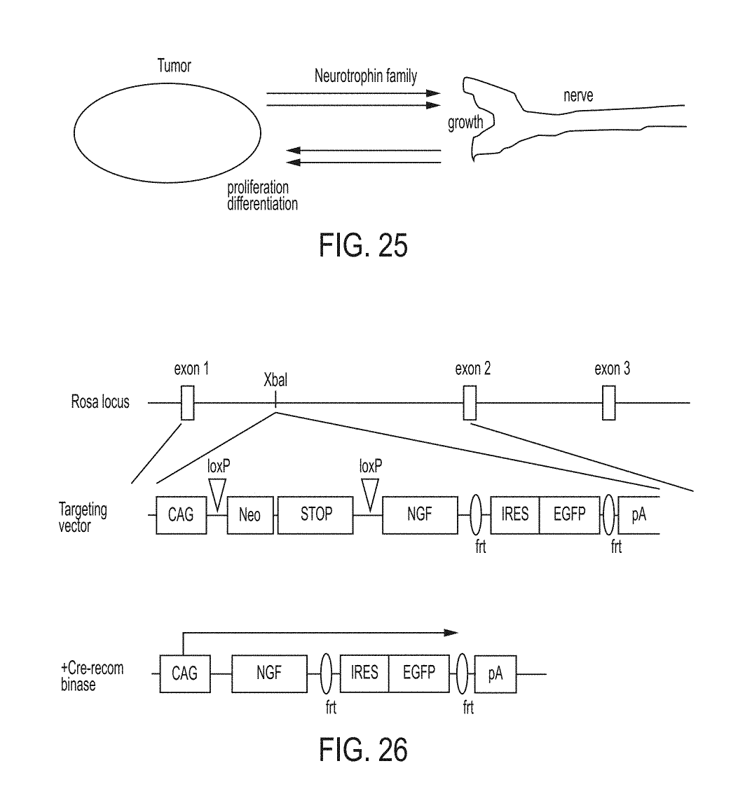

Tumors secreted neurotrophins that lead to growth of nerve axons towards the tumor (FIG. 25). Nerves then secrete neurotransmitters and/or growth factors that stimulate proliferation of tumors.

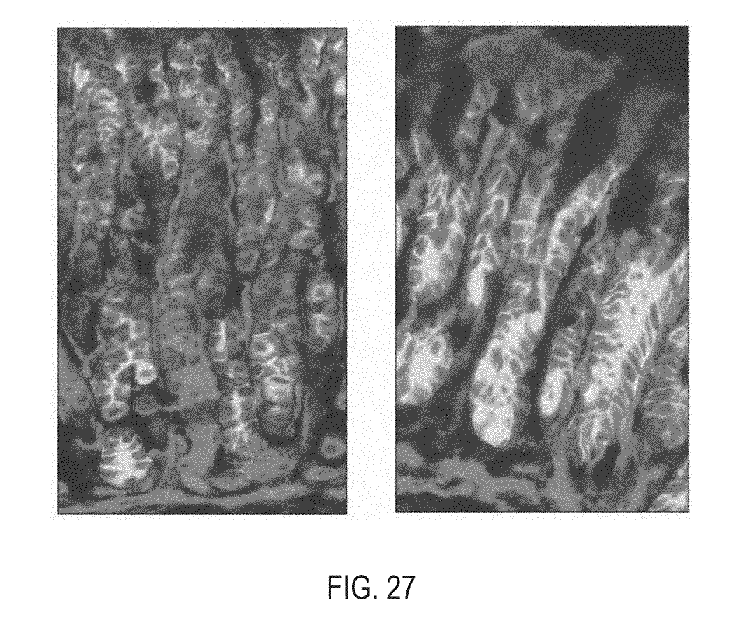

Conditional NGF overexpression mice were generated (FIG. 26). The construct CAG-LSL-NGF-IRES-EGFP was generated through recombineering of the Rosa26 BAC and then inserted into the mouse germline. Expression of NGF and EGFP is prevented by a stop codon surrounded by 1oxP sites. By mating with Cre-expressing transgenic mice, the Stop sequence is removed and NGF will be expressed in specific cell types. The TFF2-Cre (line3a) was crossed to Rosa26-mTmG (Tomato-GFP). Cre recombination leads to loss of tomato (red) expression and activation of GFP expression. FIG. 27 shows entire gastric glands in the fundus (left) and antrum (right) are GFP positive. Thus, TFF2-Cre targets all gastric epithelial cells.

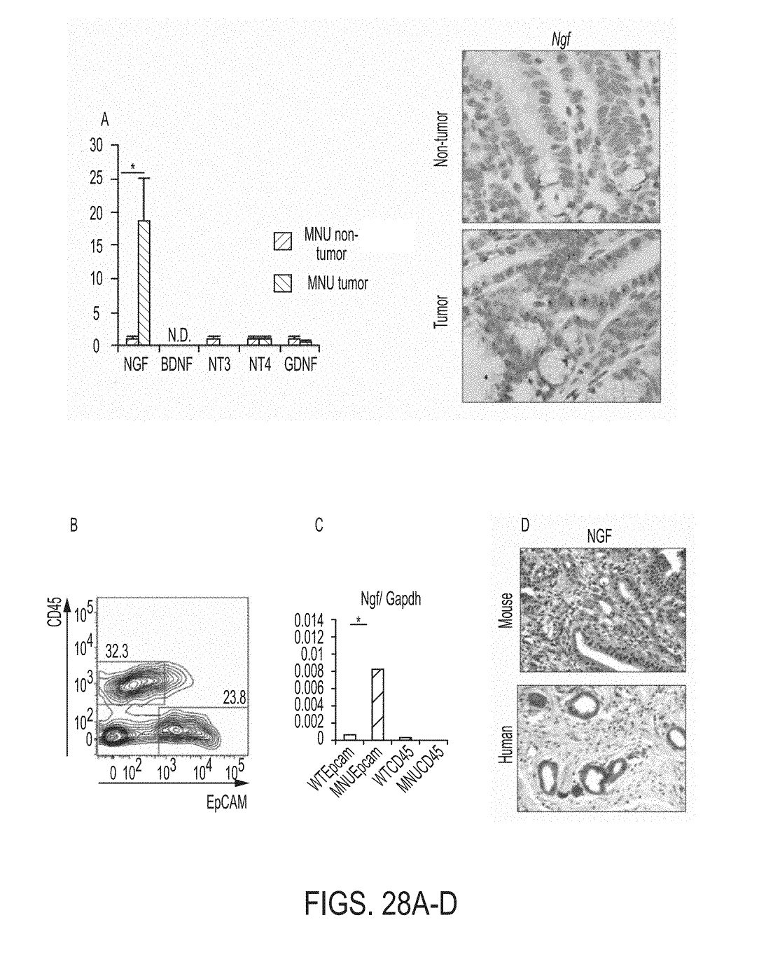

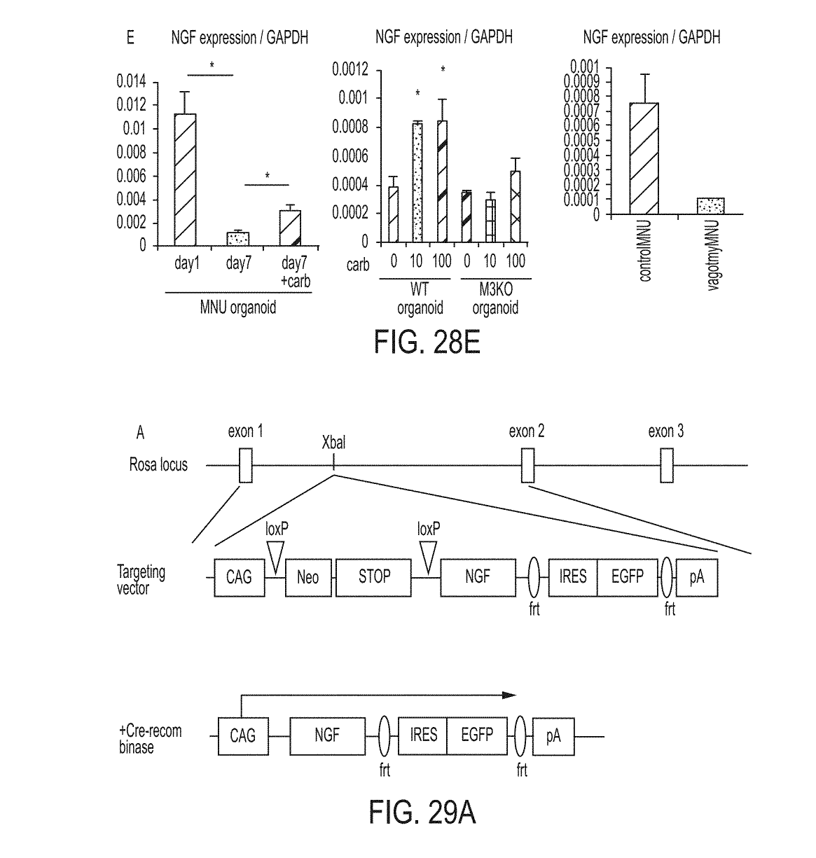

FIGS. 28A-E show that NGF is upregulated in gastric cancers and NGF is expressed in cancer cells. NGF expression in cultured gastric organoid from MNU tumor is decreased at day 7 compared to day 1, but is upregulated by treatment with carbachol. (Middle) NGF expression in cultured gastric organoids from WT and M3 Receptor Knockout (M3RKO) mice. NGF is upregulated by carbachol treatment, but shows no change in M3RKO organoids. Vagotomy downregulated NGF expression in tumors.

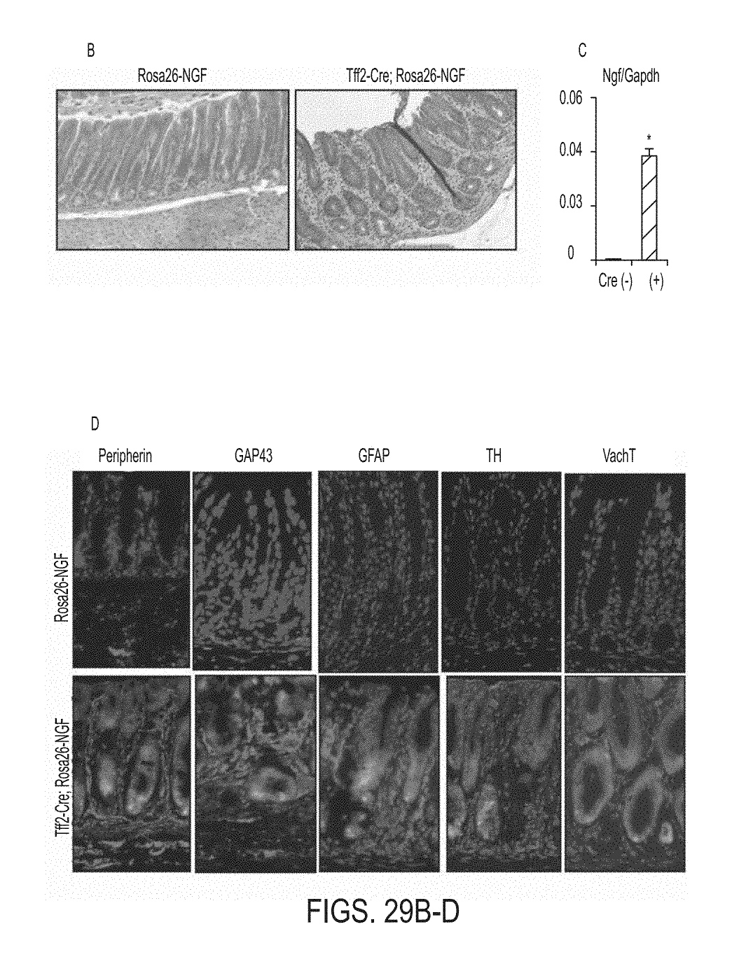

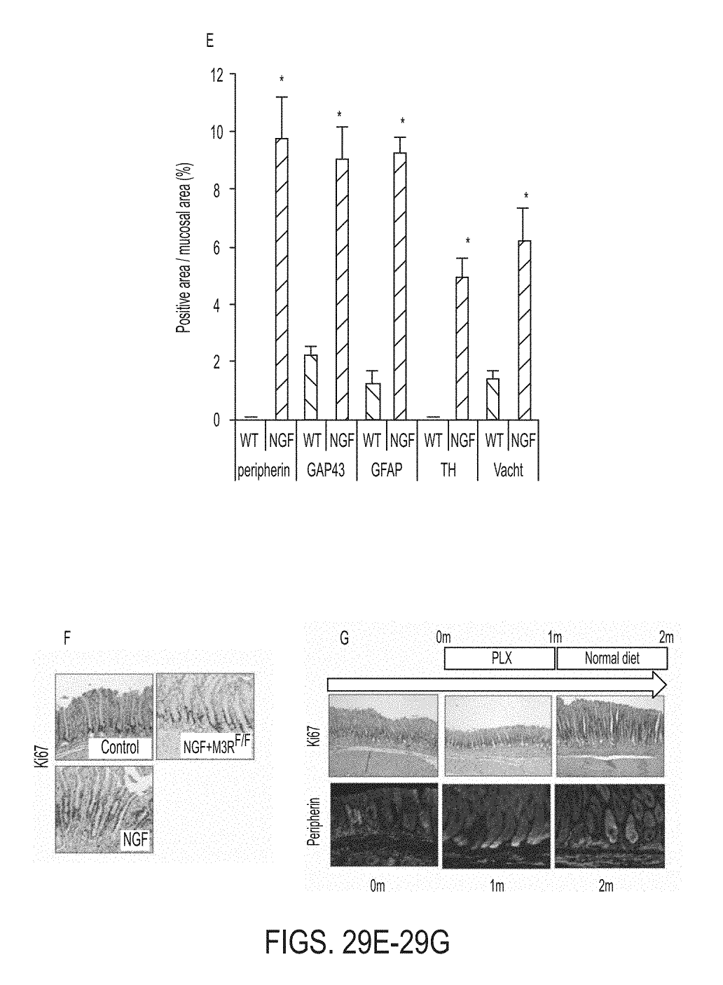

NGF/Trk signaling regulates mucosal innervation and proliferation (FIGS. 29A-G). NGF overexpression leads to abnormal gland structure. Ki67 staining showing gastric mucosal proliferation in control (WT) mice compared to TFF2-Cre; R26-LSL-NGF (or NGF) mice, compared to TFF2-Cre;R26-LSL-NGF;M3R F/F mice. The latter mice also have a conditional knockout of the muscarinic-3 (M3) receptor and thus no longer responded to cholinergic stimulation driven by NGF overexpression. NGF overexpression promotes proliferation but it is blocked by M3R knockout. TFF2-Cre;R26-LSL-NGF mice were put on a normal diet up until 3 months of age, and then treated with the TRK inhibitor (PLX7486) in their mouse chow for one month, and then put on a normal diet again. TRK inhibition completely reversed the neurogenesis and proliferation due to NGF overexpression in the stomach, but the inhibitory effect was reversible and lost when the mice resumed a normal diet.

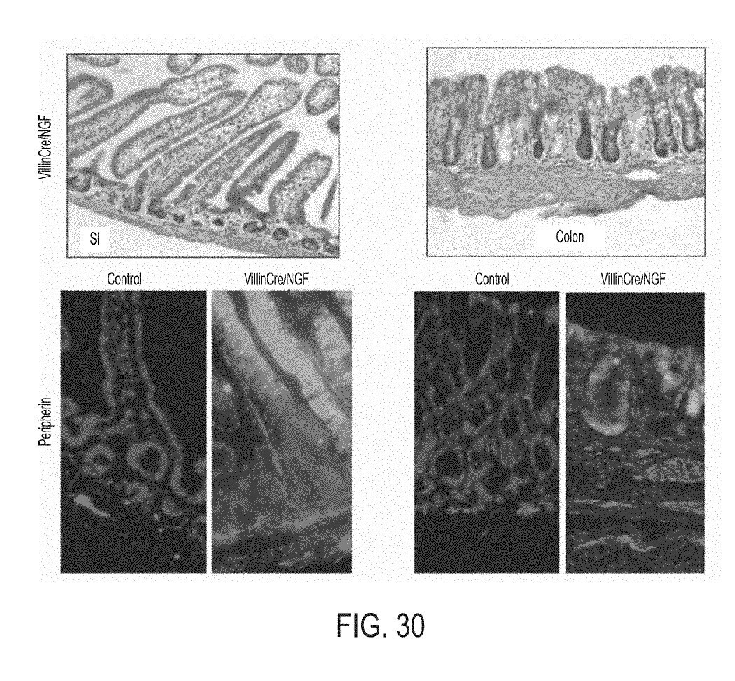

NGF overexpression in the small intestine and colon leads to marked innervation (FIG. 30). Increased innervation, as manifested by red peripherin staining, is seen in the NGF-overexpressing mouse intestine and colon.

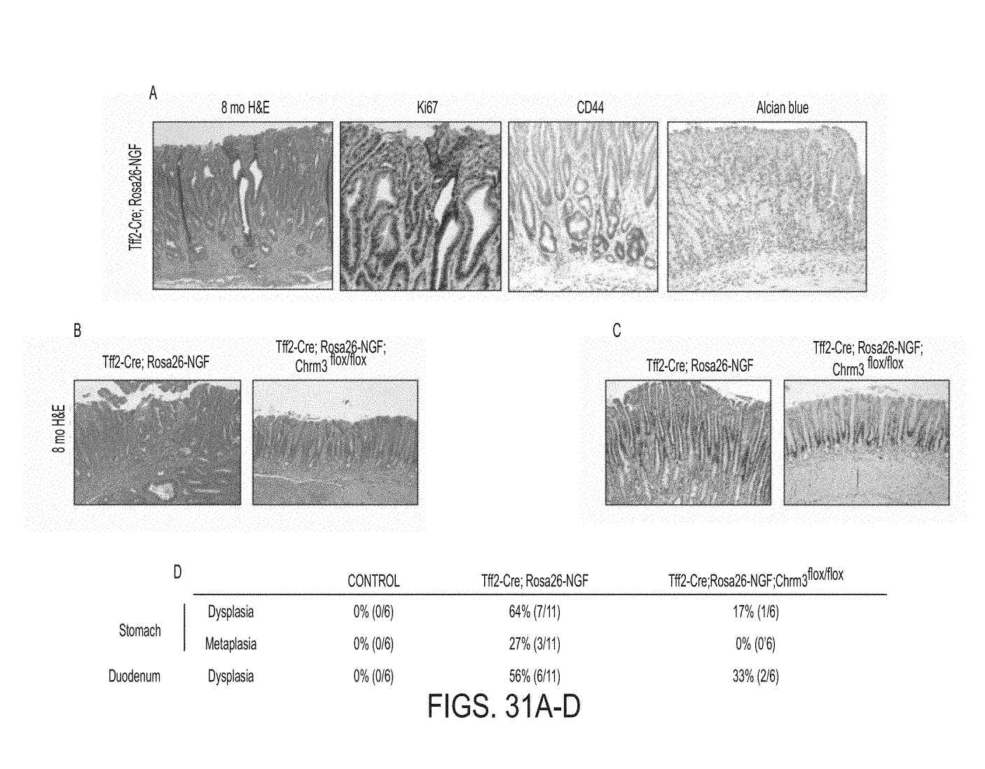

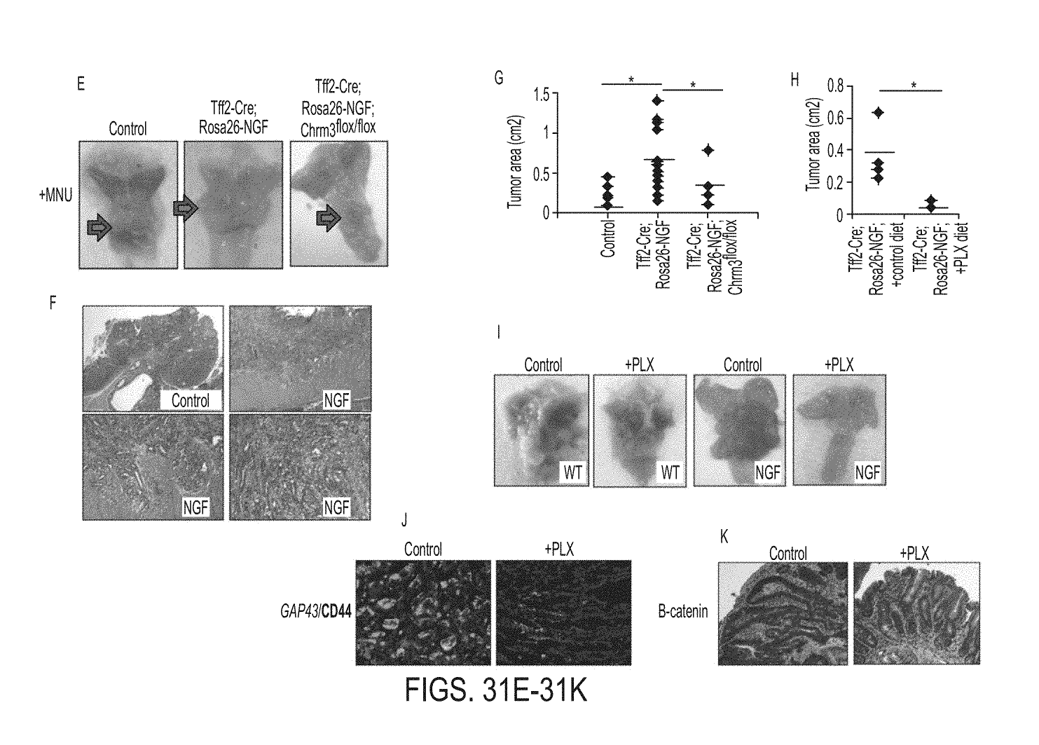

Abnormal cholinergic innervation promotes gastric proliferation and carcinogenesis (FIGS. 31A-K). 8 mo old Tff2-Cre; R26-NGF mice develop spontaneous dysplasia. M3R knockout blocks the development of dysplasia. NGF overexpression promotes MNU induced tumor development with a more aggressive histopathology. M3R knockout blocks this effect. PLX7486 prevented tumor growth in both WT and Tff2-Cre; R26-NGF mice. PLX7486 decreased nerve density, the number of CD44+ cells, and the number of beta-catenin+ cells.

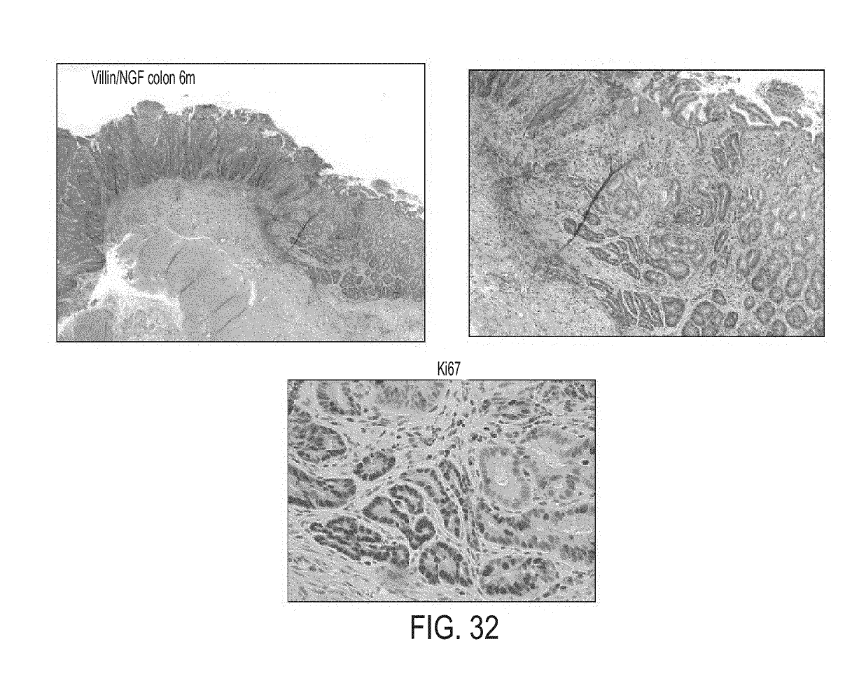

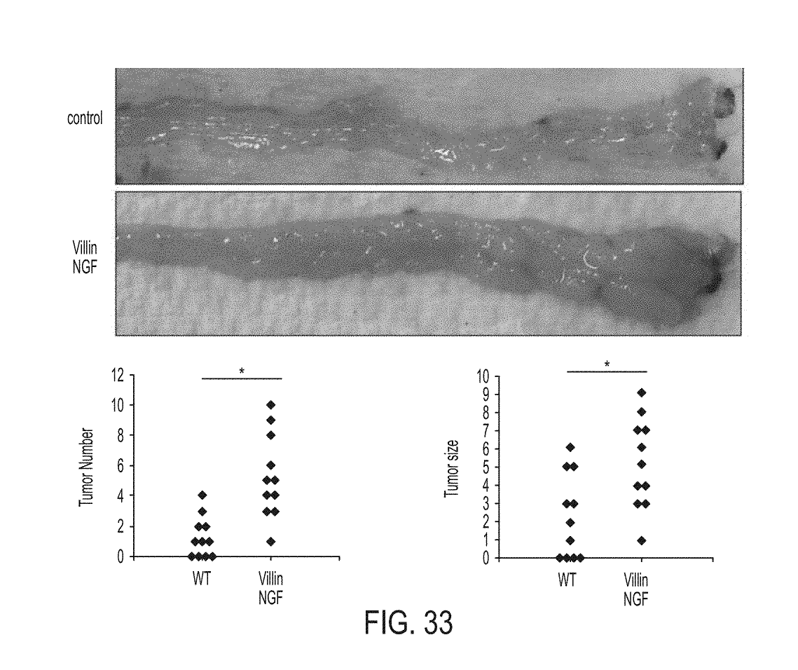

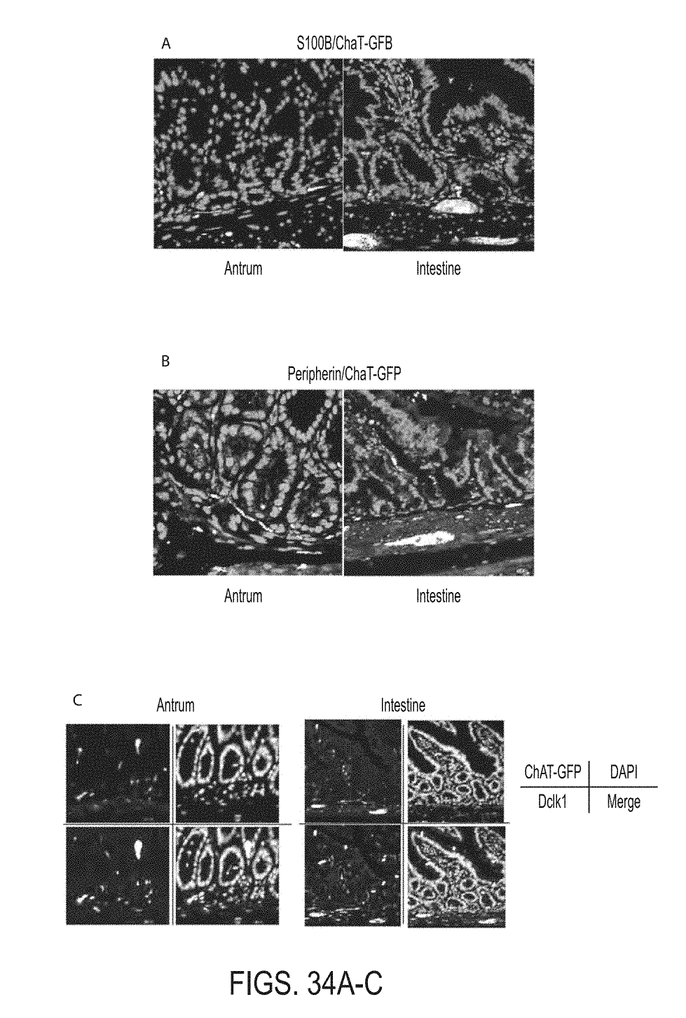

Villin-Cre; NGF mice develop colonic dysplasia (FIG. 32). Villin-Cre; NGF mice are more susceptible to AOM-DSS tumor (FIG. 33). In the AOM-DSS colon cancer model, villinCre;NGF mice show increased number and size of colonic tumors.

ChaT (choline acetyltransferase, which produces acetylcholine) is expressed in enteric ganglia, mucosal nerve fibers, and scattered rare epithelial cells in the gut (FIGS. 34A-C). Dclk1 is also expressed in ChaT+ ganglia and nerves, suggesting that Dclk1 +cells mark the source of Ach in the gut.

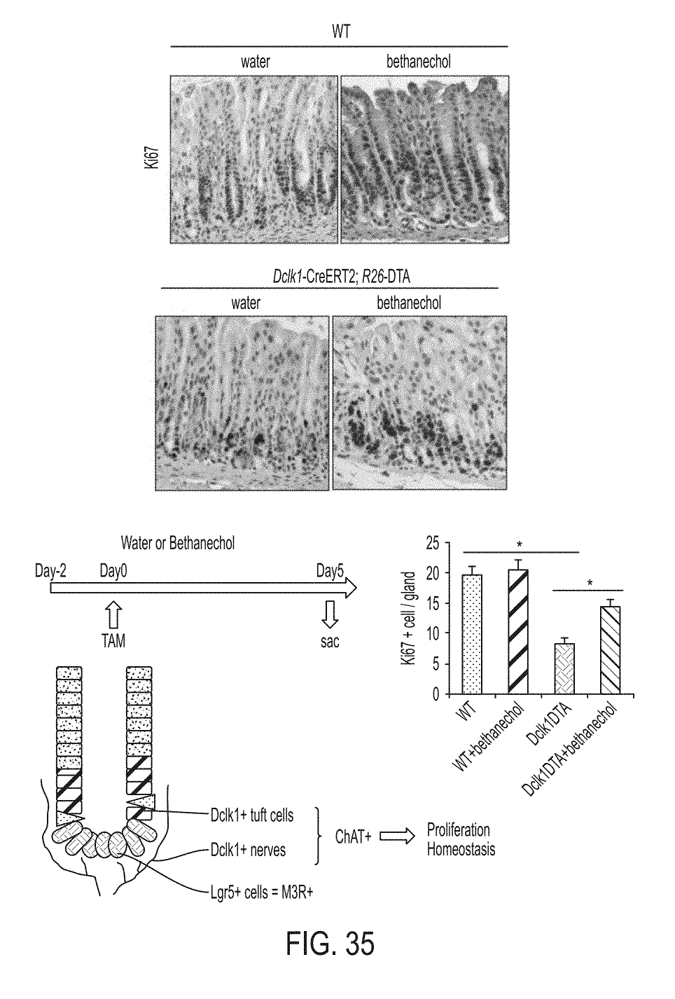

Ablation of Dclk1 +cells leads to a decrease of proliferation in the stomach (FIG. 35). Treatment with bethanechol can rescue this effect at least in part. Dclk1 +tuft cells and nerves seem to regulate mucosal homeostasis through cholinergic signal, possibly acting on Lgr5+ stem cells expressing the M3 receptor.

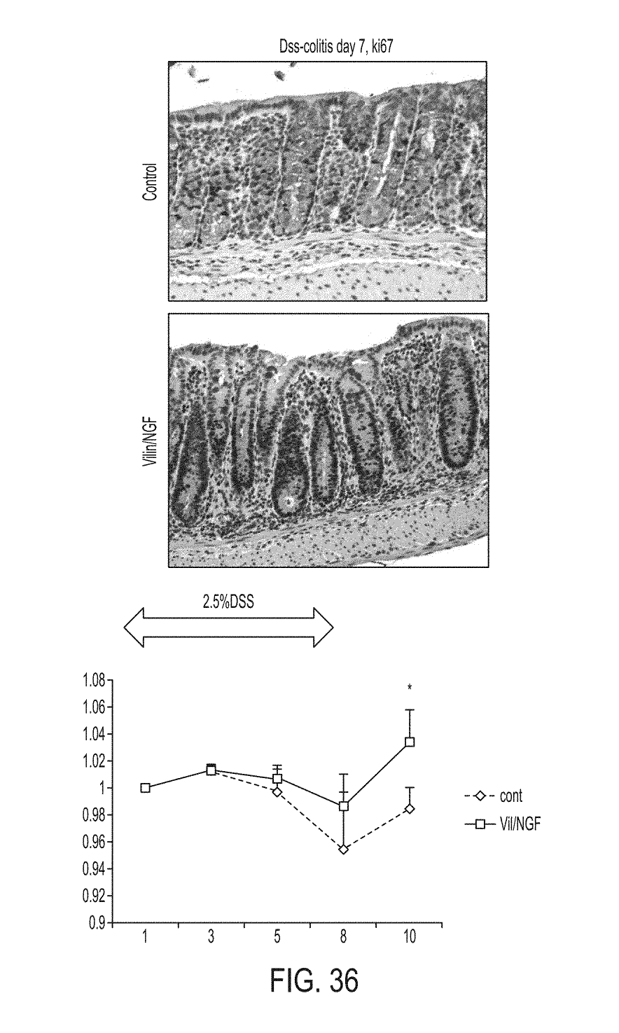

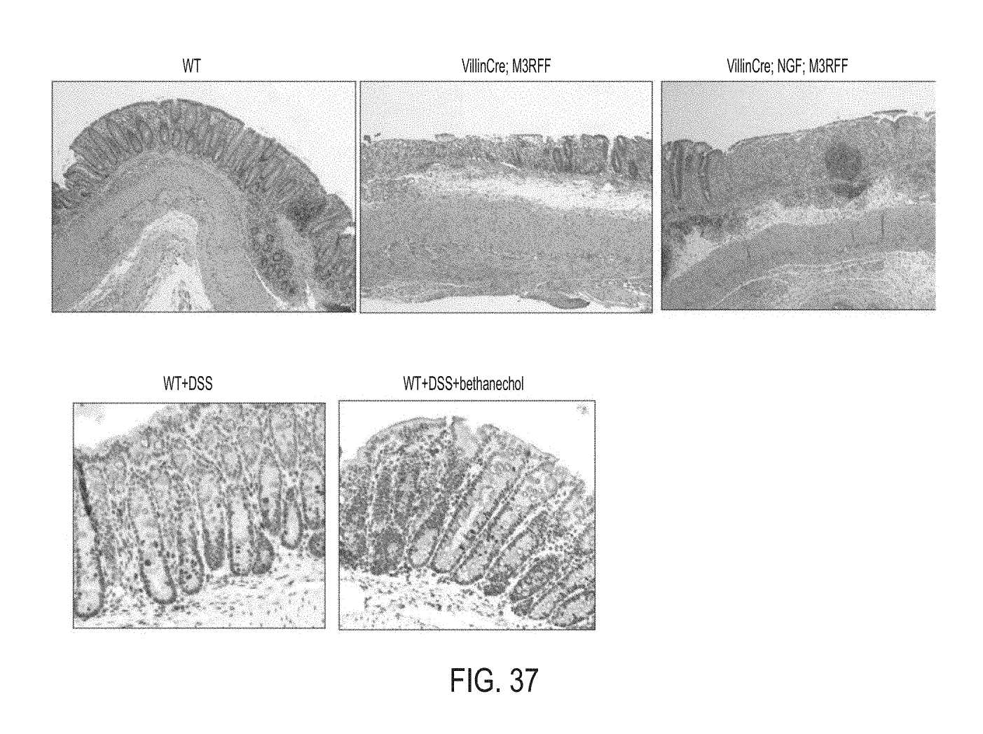

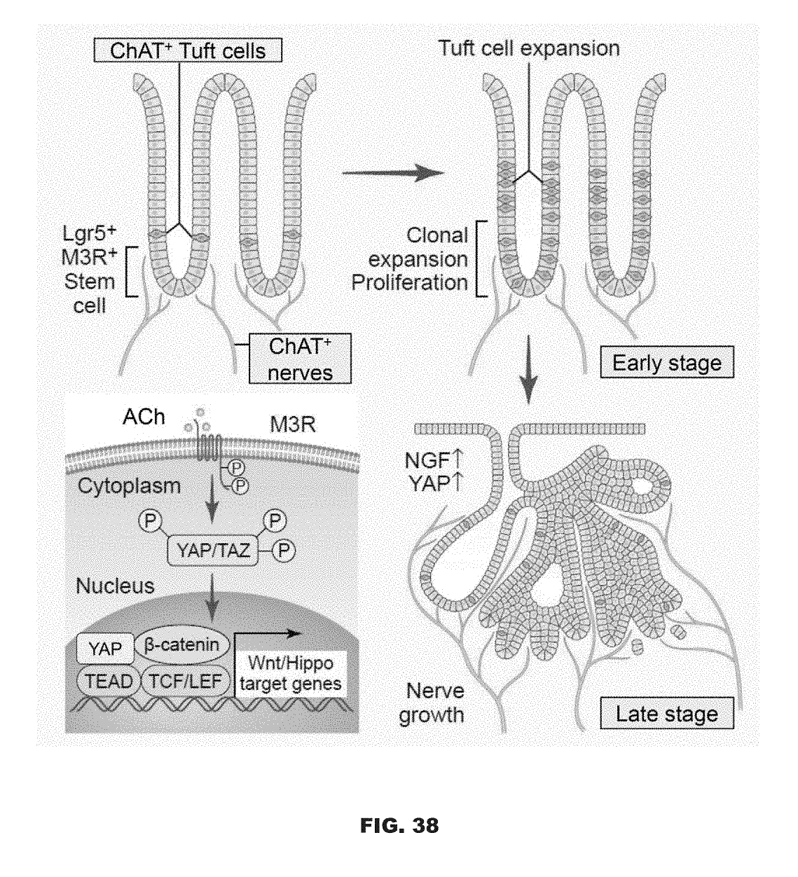

Villin-NGF mice are resistant to colonic injury (FIG. 36). VillinCre;NGF mice are more resistant to DSS-colitis compared to control mice, and they show less weight loss and have more proliferating cells in the mucosa. Cholinergic signaling promotes mucosal regeneration and healing in colitis (FIG. 37). Deletion of M3R in colonic mucosa leads to more severe colitis, even in NGF overexpression mice. Treatment with bethanechol increased proliferation in DSS-treated WT mice. Ach/M3R signaling promotes colonic regeneration after DSS.

### EXAMPLE 3

### Nerve Growth Factor Promotes Gastric Tumorigenesis through Aberrant Cholinergic Signaling

Described herein is the use of a series of mouse models to show that acetylcholine from Dclk1 +tuft cells and nerves induces NGF in gastric epithelial cells, which promotes neuron expansion and tumorigenesis. YAP is activated through the cholinergic signaling, and inhibition of this pathway can block NGF-driven tumors.

The results decribes herein show that: (i) NGF expression is induced in gastric cancer by ACh from nerves and tuft cells; (ii) NGF promotes innervation and proliferation in gastric epithelium; (iii) blockade of NGF or ablation of cholinergic tuft cells inhibits tumor development; and (iv) cholinergic signaling activates YAP signaling that is essential for Wnt activation.

Summary and Significance

Within the gastrointestinal stem cell niche, nerves help to regulate both normal and neoplastic stem cell dynamics. Here, the mechanisms underlying the cancer-nerve partnership are revealed. It was found that Dclk1 +tuft cells and nerves are the main sources of acetylcholine (ACh) within the gastric mucosa. Cholinergic stimulation of the gastric epithelium induced nerve growth factor (NGF) expression, and in turn NGF overexpression within gastric epithelium expanded enteric nerves and promoted carcinogenesis. Ablation of Dclk1 +cells or blockade of NGF/Trk signaling inhibited epithelial proliferation and tumorigenesis in an ACh muscarinic receptor-3 (M3R)-dependent manner, in part through suppression of yes-associated protein (YAP) function. This feedforward ACh-NGF axis activates the gastric cancer niche and offers a compelling target for tumor treatment and prevention.

The factors driving nerve expansion during tumorigenesis and the downstream targets of nerve signaling are not well understood. This study investigates the extensive crosstalk that occurs during carcinogenesis between cancer cells and nerves, and identifies the ACh-NGF-M3R-YAP axis that is central to gastric cancer biology. This work proposes potential targets for cancer therapy, such as NGF and M3R, which can be clinically applied.

Introduction

There has been considerable interest in the biologic and therapeutic implications of neural regulation of normal stem cells and cancer growth (Brownell et al., 2011; Hanoun et al., 2014; Katayama et al., 2006; Magnon et al., 2013; Mendez-Ferrer et al., 2010; Peterson et al., 2015; Stopczynski et al., 2014; Venkatesh et al., 2015; Zhao et al., 2014). In the gastrointestinal tract, acetylcholine (ACh) regulates epithelial stem cells, proliferation, and tumorigenesis via the muscarinic receptor-3 (M3R), in part through modulation of Wnt signaling (Lundgren et al., 2011; Raufman et al., 2008; Zhao et al., 2014). Canonical Wnt activation is characterized by nuclear translocation of b-catenin leading to activation of the transcriptional factor T cell factor (TCF) family and target gene expression. However, to achieve TCF activation, multiple transcriptional co-activators are required, including the yes-associated protein (YAP) (Rosenbluh et al., 2012), which appears to form an important part of the ACh-M3R axis.

Although many cancers, including stomach, pancreas, and colon, show increased nerve density (Albo et al., 2011; Ceyhan et al., 2010; Zhao et al., 2014), the overall significance of tumor-associated neural plasticity remains uncertain. The neurotrophin family molecules signal through Trk receptors to support neuron survival and axonal growth. In cancer, there is often an upregulation of neurotrophins or Trk receptors to activate cancer cell proliferation in an autocrine manner (Dolle et al., 2004; Weeraratna et al., 2001). Trk inhibitors that suppress neurotrophin signaling have been used in the treatment of cancers characterized by an activated Trk fusion protein (Vaishnavi et al., 2015). Given the relevance of neurotrophin/Trk signaling in neural development, and the possible importance of this pathway in cancer signaling, it was investigated whether neurotrophin/Trk signaling might represent a potent driver of peritumoral innervation and tumor growth.

The enteric nervous system (ENS) has an ability to regulate gastrointestinal homeostasis through direct innervations to gastrointestinal crypts (Gross et al., 2012; Neal and Bornstein, 2007), which appears to be linked to epithelial homeostasis. For example, sympathetic nerves accelerate crypt cell proliferation through norepinephrine (Tutton and Helme, 1973), and serotonin from ENS components promotes growth and turnover of the mucosal epithelium, by regulating muscarinic cholinergic innervation to epithelial effectors (Gross et al., 2012; Tutton and Barkla, 1986). Although the role of cholinergic signaling in gut proliferation and cancer has been suggested, the precise molecular mechanism in the ENS-cancer interaction remains uncertain.

Nerves also promote mucosal regeneration indirectly via Dclk1 ⁻tuft cells. Dclk1 ⁺tuft cells act, in part, as intermediary niche cells coordinating neural input to help regulate subsequent stem cell activity (Chandrakesan et al., 2015; Westphalen et al., 2014). Tuft cells express choline-acetyltransferase (ChAT), the enzyme responsible for ACh production, and they have a neuron-like gene expression signature (Schutz et al., 2015). Tuft cells also express cytokines such as interleukin-25 and cyclooxygenase-2, and help to mediate inflammatory responses within gastrointestinal mucosa (Bezencon et al., 2008; von Moltke et al., 2016). Given their unique nature, it was investigated whether tuft cells could help coordinate the crosstalk between nerves and cancer. Accordingly, described herein is a study to reveal the whole picture of the nerve-cancer interaction during tumorigenesis with multiple mouse models.

Results

ChAT⁺ Tuft Cells and Nerves Expand within Gastric Mucosa during Tumorigenesis

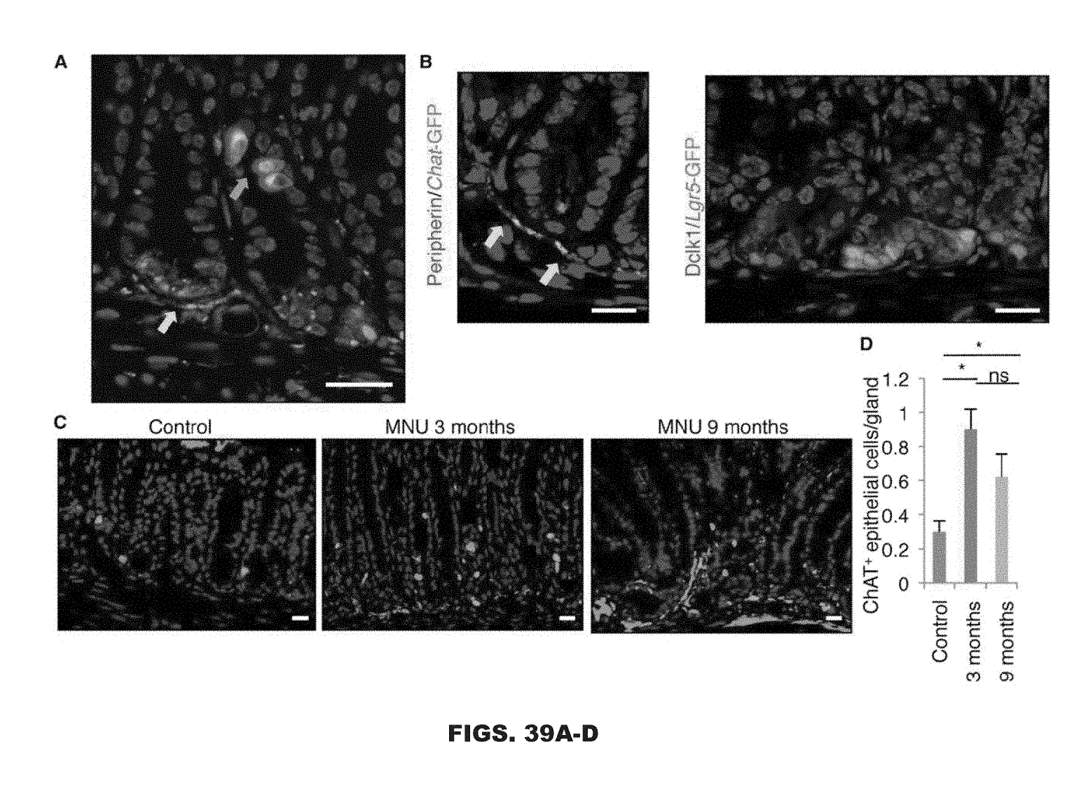

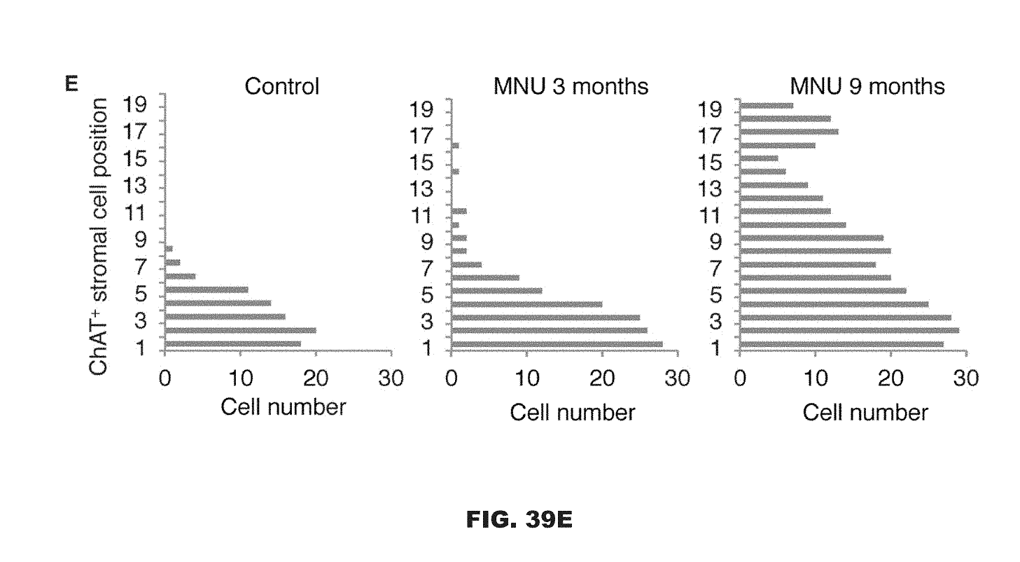

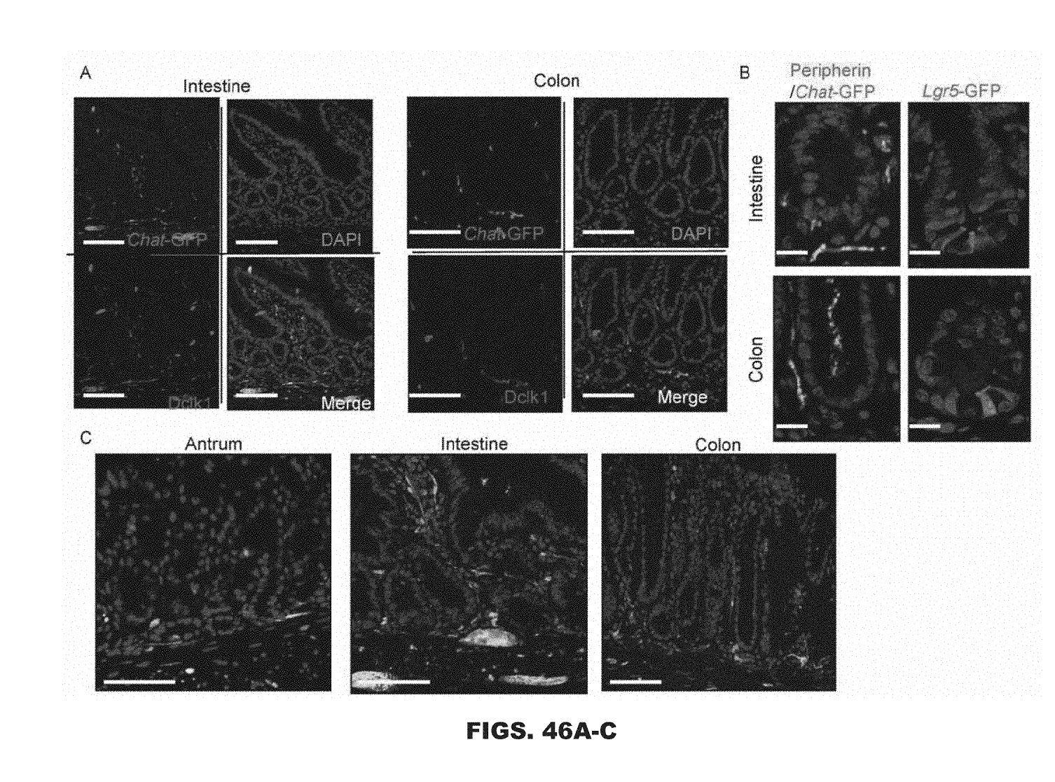

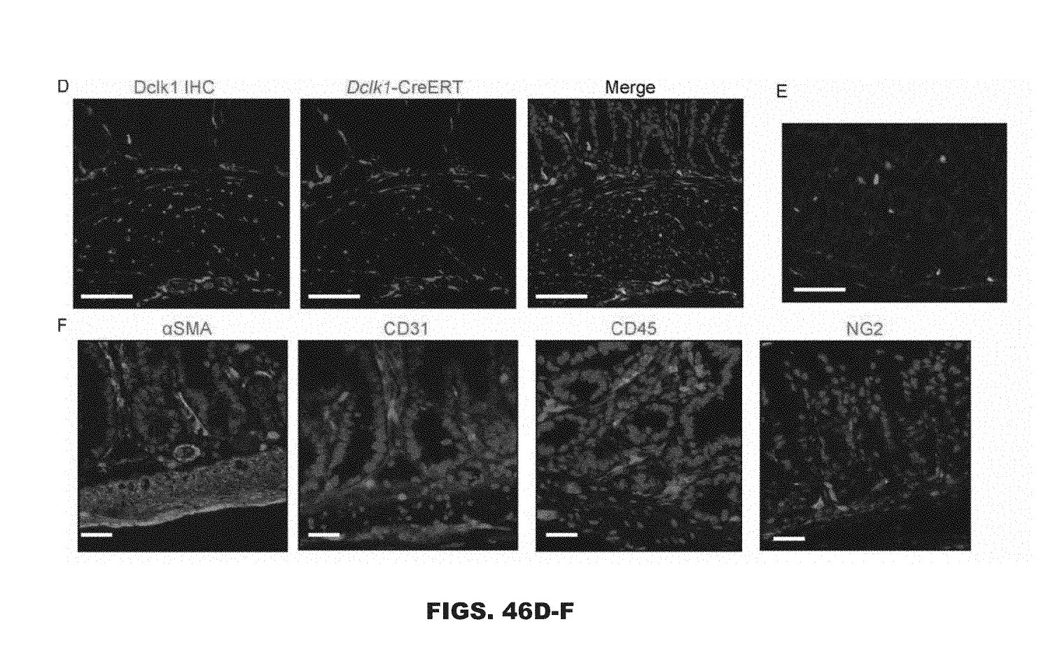

The source of ACh within the alimentary tract was explored using Chat-GFP transgenic mice, in which all ACh-producing cells are GFP⁺ (Tallini et al., 2006). Chat-GFP is expressed in nerve fibers within the lamina propria and the submucosal and myenteric ganglia (FIGS. 39A and 46A). GFP⁺ nerve fibers surround the base of glands, where stem cells such as Lgr5⁺ cells reside, supporting the notion that cholinergic nerves contribute to the gastrointestinal stem cell niche through close physiological contact (FIGS. 39B, 46B, and 46C). As shown previously (Schutz et al., 2015), Chat-GFP is also expressed in epithelial tuft cells that are positive for Dclk1 (FIGS. 39A and 46A). Immunostaining revealed that Dclk1 is strongly expressed in tuft cells, but also detected mild-to-moderate Dclk1 expression in Chat-GFP⁺ cholinergic nerve fibers and ganglia (FIGS. 39A, 39B, and 46A). Dclk1-CreERT mice (Westphalen et al., 2014) confirmed Dclk1 expression in a subset of ENS as well as epithelial tuft cells, and both of which showed immunopositivity for ACh (FIGS. 46D and 46E). However, Dclk1 is not expressed in other stromal lineages, such as a-smooth muscle actin (SMA)⁺ myofibroblasts, CD31⁺ endothelial cells, CD45+ hematopoietic cells, or NG2⁺ pericytes (FIG. 46F). Taken together, these results suggest that expression of Dclk1 identifies most, if not all, cholinergic signaling cells within the gut, including both epithelial tuft cells and stromal neurons.

It was previously reported that Lgr5⁻ gastric stem cells express high levels of M3R and expand in response to cholinergic signaling during carcinogenesis (Zhao et al., 2014). Interestingly, in an N-nitroso-N-methylurea (MNU) carcinogen mouse model of gastric cancer, it was found that a dynamic relationship between epithelium and stroma in terms of cholinergic cell distribution during tumor development. Early in the model (first 3 months after MNU treatment), there was a significant increase in Chat-GFP tuft cells in the epithelium. But, after 9 months, there was a gradual loss of epithelial Chat-GFP⁺ tuft cells, accompanied by axonogenesis of cholinergic nerve fibers (39C-39E). Thus, the early expansion of Char⁺ tuft cells during carcinogenesis, followed by the later increase in cholinergic innervation with progression to dysplasia, suggests a requirement for ACh production in tumorigenesis from different sources, depending on tumor stage.

ACh Signaling Stimulates NGF Production in Gastric Epithelial Cells

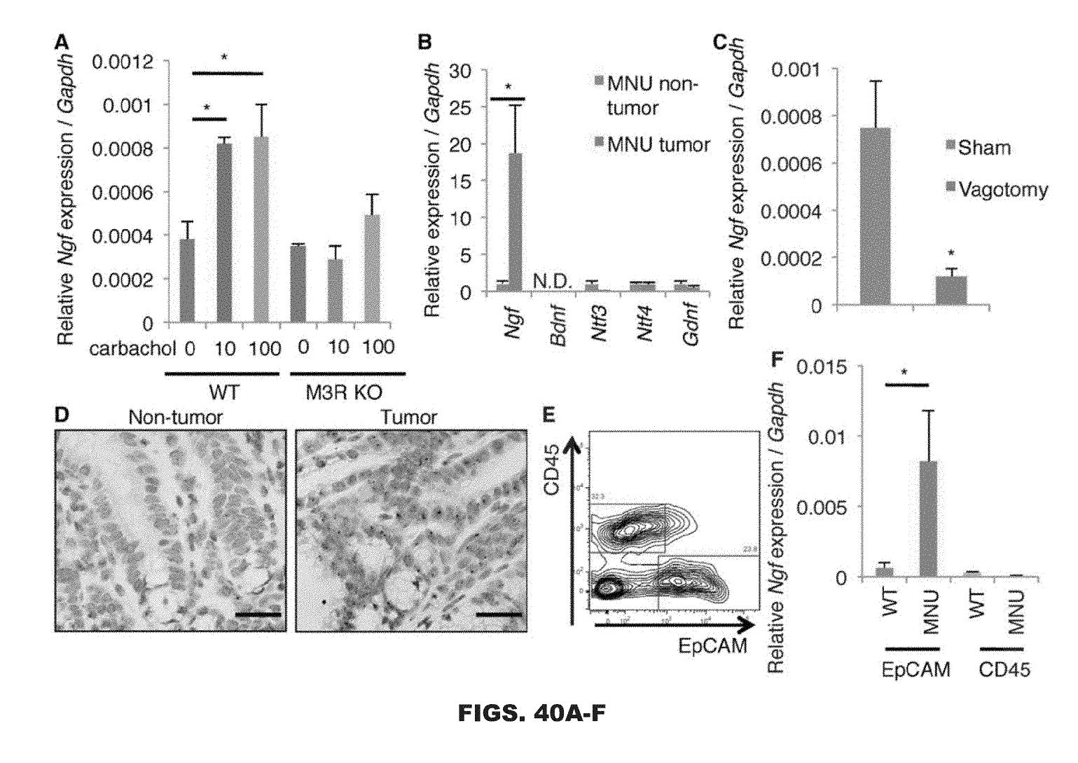

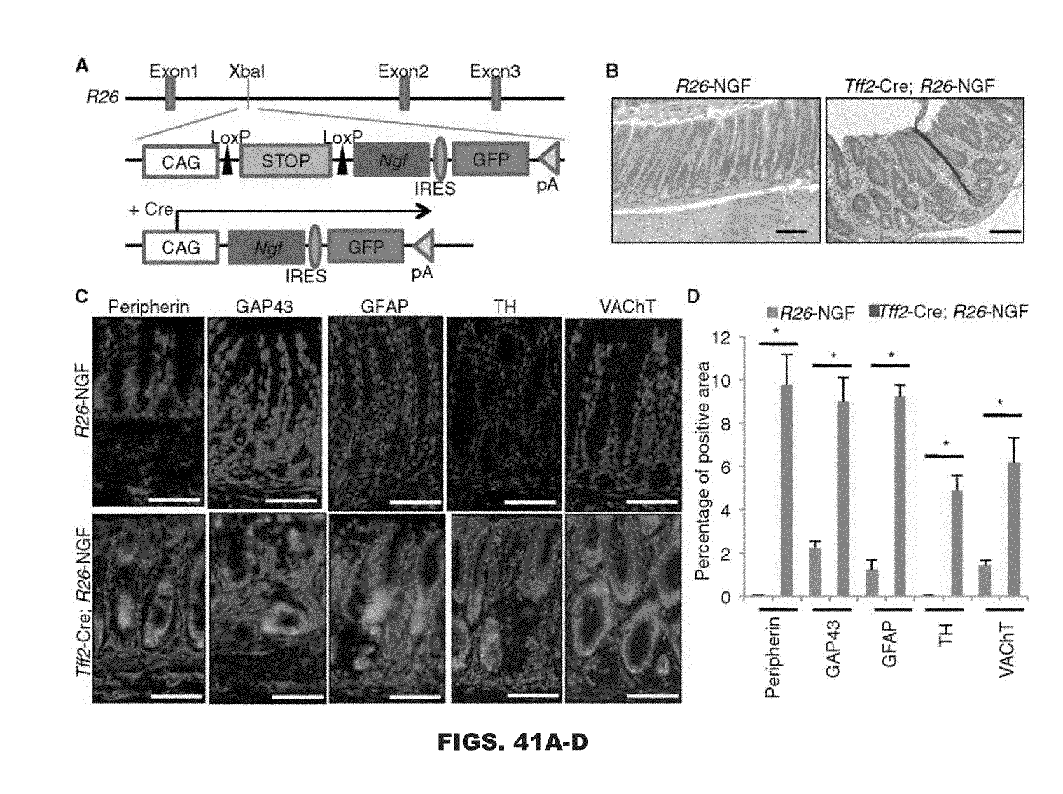

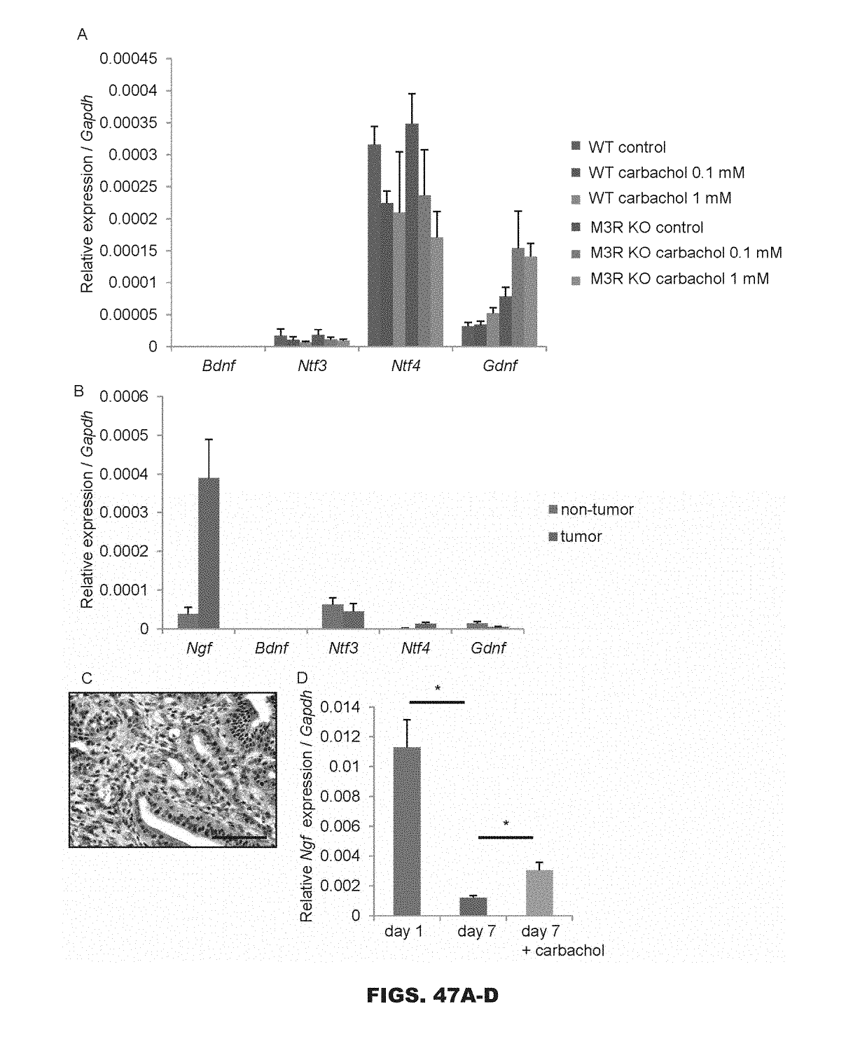

Cholinergic stimulation has been shown to induce the expression of certain neurotrophin family molecules (da Penha Berzaghi et al., 1993; Lapchak et al., 1993; Mahmoud et al., 2015). In support of these observations, treatment of gastric organoids with the cholinergic agonist carbachol upregulated the expression of Ngf in an M3R-dependent fashion (FIG. 40A). Of all the major neurotrophins (nerve growth factor [NGF], brain-derived neurotrophic factor, neurotrophin 3, neurotrophin 4, and glial cell line-derived neurotrophic factor), NGF was most highly and specifically upregulated by carbachol (FIGS. 40A and 47A). In mouse gastric tumors, there was also a specific upregulation of Ngf (almost 20 times higher expression than the normal stomach), and such upregulation was not observed in any other neurotrophins (FIG. 40B). Similar upregulation of Ngf was found in mouse colorectal tumors induced by azoxymethane (AOM) and dextran sodium sulfate (DSS) (FIG. 47B). Interestingly, abrogation of cholinergic signaling in the stomach by surgical vagotomy was able to inhibit Ngf upregulation in gastric tumors (FIG. 40C). Immunostaining and in situ hybridization confirmed that NGF was expressed within the neoplastic epithelium rather than the stromal compartment (FIGS. 40D and 47C), a finding confirmed by cell sorting and subsequent qPCR (FIGS. 40E and 40F). MNU tumor-derived organoids that are cultured in isolation, separated from their native microenvironment, lost Ngf expression within 7 days. However, treatment with the ACh mimetic carbachol partly reestablished Ngf upregulation (FIG. 47D). Therefore, ACh production, possibly from tuft cells initially but later from innervated nerves with axonogenesis, is at least partly responsible for the neoplastic upregulation of NGF.

NGF/Trk Signaling Regulates Mucosal Innervation

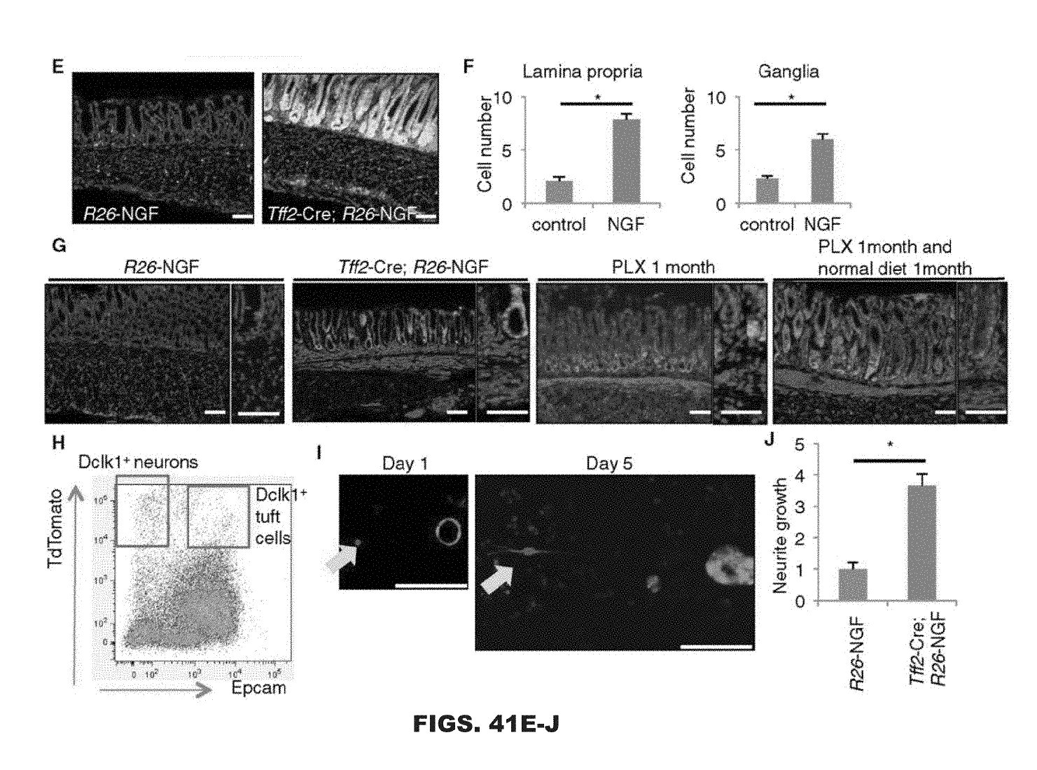

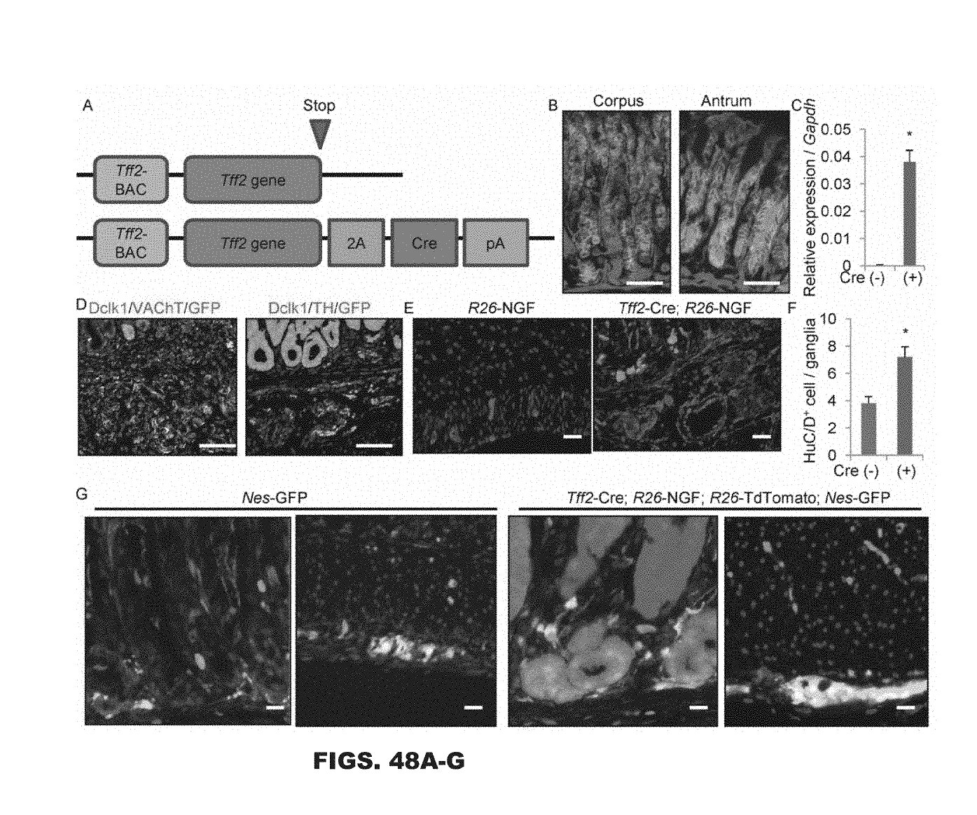

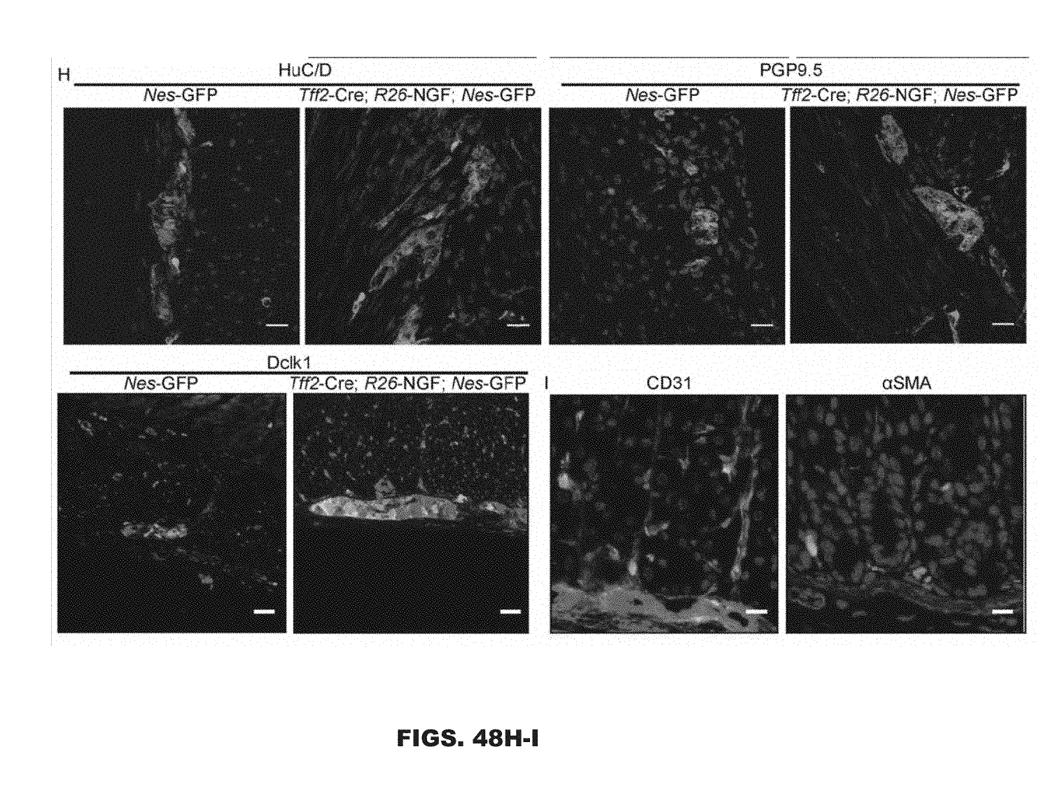

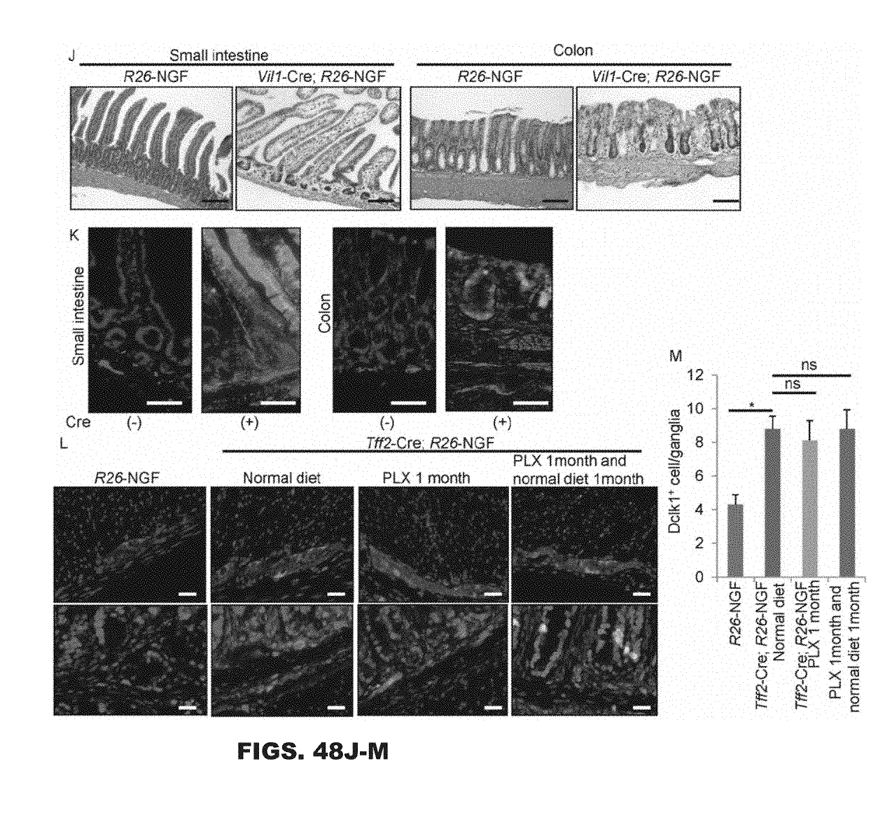

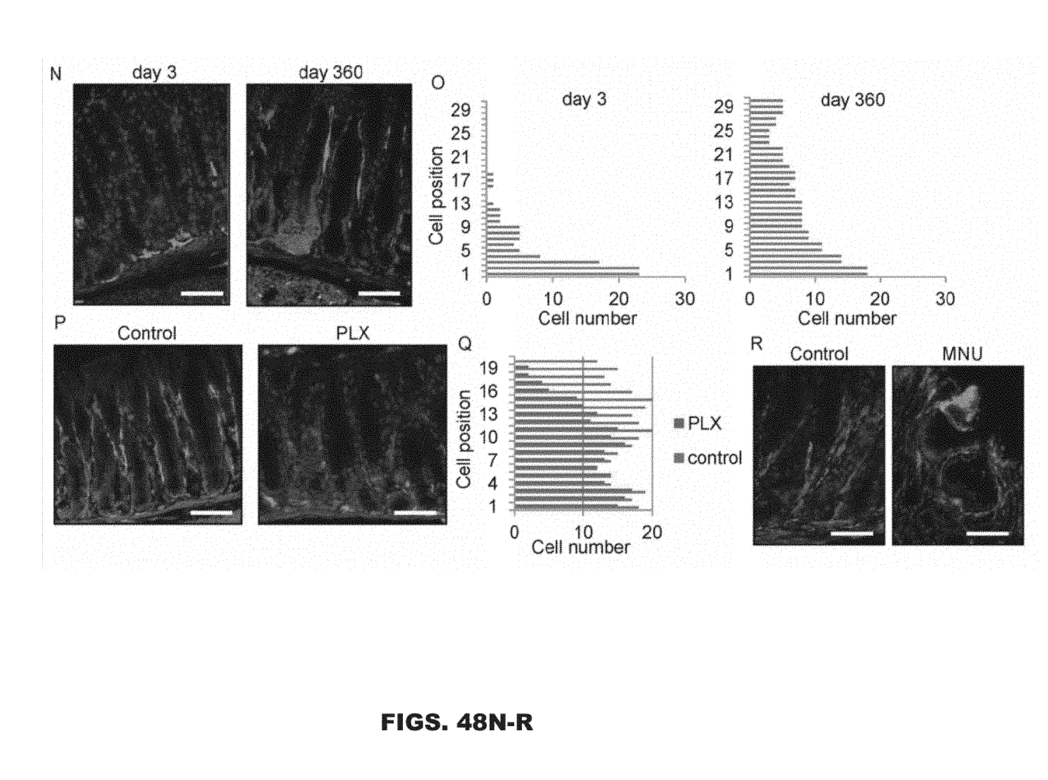

Based on the theory that NGF plays a role in tumor-associated innervation, knockin mice which conditionally express mouse Ngf gene were generated to test this in vivo. To create this line, a Lox-STOP-Lox-Ngf-IRES-GFP construct was inserted into the Rosa26 (referred to as R26) gene locus (FIG. 41A). To target NGF expression to the gastric epithelium, a Tff2-BAC-Cre line was used (Dubeykovskaya et al., 2016), which targeted Cre recombinase expression to the entire gastric epithelium (FIGS. 48A and 48B). Tff2-Cre; R26-NGF mice expressed a high level of Ngf in the gastric epithelium (FIG. 48C), leading to disturbed glandular architecture and increased stromal cells within the lamina propria (FIG. 41B). Immunostaining revealed that these stromal cells were nerves and glial cells, including both adrenergic (TH⁺) and cholinergic (vesicular ACh transporter [VAChT]⁺) neurons that expressed Dclk1 (FIGS. 41C, 41D, and 48D). It was also found that submucosal ganglia were larger in NGF-overexpressing mice and indeed the number of HuC/D⁺ cells in ganglia was significantly increased (FIGS. 48E and 48F). In addition, Nestin⁺ cells were expanded within the lamina propria and submucosal ganglia in NGF-overexpressing mice (FIGS. 41E and 41F). As reported previously (Belkind-Gerson et al., 2013; Grundmann et al., 2016), the majority of these Nestin⁺ cells are positive for a glial marker S100B, thus including glial progenitors and mature glia, but negative for neuronal markers such as HuC/D, PGP9.5, and Dclk1, or other stromal markers including CD31 and a-SMA (FIGS. 48G-48I). Targeting NGF overexpression to the small and large intestine using crosses to Vil1-Cre transgenic mice increased nerve density in these organs (FIGS. 48J and 48K). The expansion of stromal nerves in NGF-overexpressing mice was suppressed by the treatment with the Trk inhibitor PLX-7486 (PLX), although the nerves rapidly re-expanded after the discontinuation of PLX treatment (FIG. 41G). In contrast, the numbers of Dclk1⁺ neuronal cells in ganglia did not change during this time course, and these cells did not show any uptake of BrdU even after 2 months continuous administration (FIGS. 48L and 48M), suggesting that the expansion of nerve fibers in NGF-overexpressing mice during adulthood occurs primarily through axonogenesis, rather than neurogenesis.

In adult Dck1-CreERT; R26-mTmG mice treated with tamoxifen, the recombined GFP⁺ nerves were initially found near the gastric gland base. However, after 1 year, these recombined cells gradually expanded toward the top of glands (FIGS. 48N and 48O), suggesting time-dependent axonal growth and/or nerve turnover under normal homeostasis, as shown in other studies (Kabouridis et al., 2015). PLX treatment suppressed this stromal lineage tracing in Dclk1-CreERT; R26-mTmG mice (FIGS. 48P and 48Q), suggesting a role for NGF in the remodeling of the stroma. Consistent with this notion, stromal tracing in Dclk1-CreERT; R26-mTmG mice was enhanced in MNU-induced tumors that expressed high levels of NGF (FIG. 48R). To further explore this relationship, we co-cultured sorted Epcam^{-Dclk}1+ neurons with wild-type (WT) or Tff2-Cre; R26-NGF gastric organoids (FIG. 41H). While cultured neurons rarely showed much neurite outgrowth when co-cultured with WT organoids, NGF-overexpressing organoids induced significant neurite growth (FIGS. 41I and 41J), mimicking the in vivo Dclk1 + axonal growth that was dependent on NGF.

ACh/M3R Signaling Regulates Mucosal Proliferation and Clonal Stem Cell Expansion

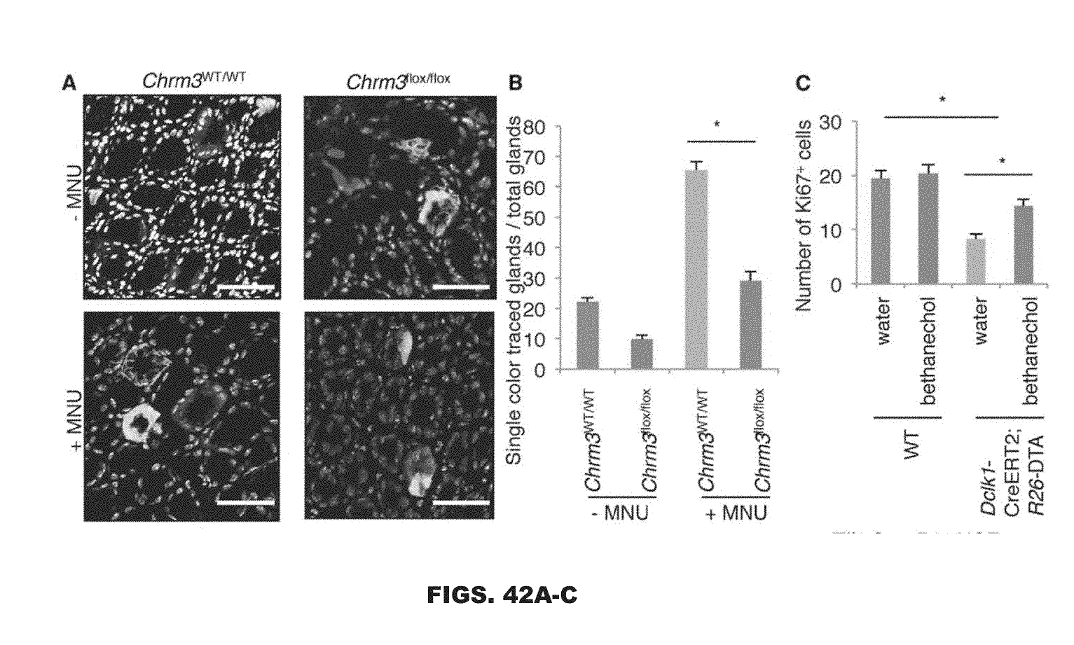

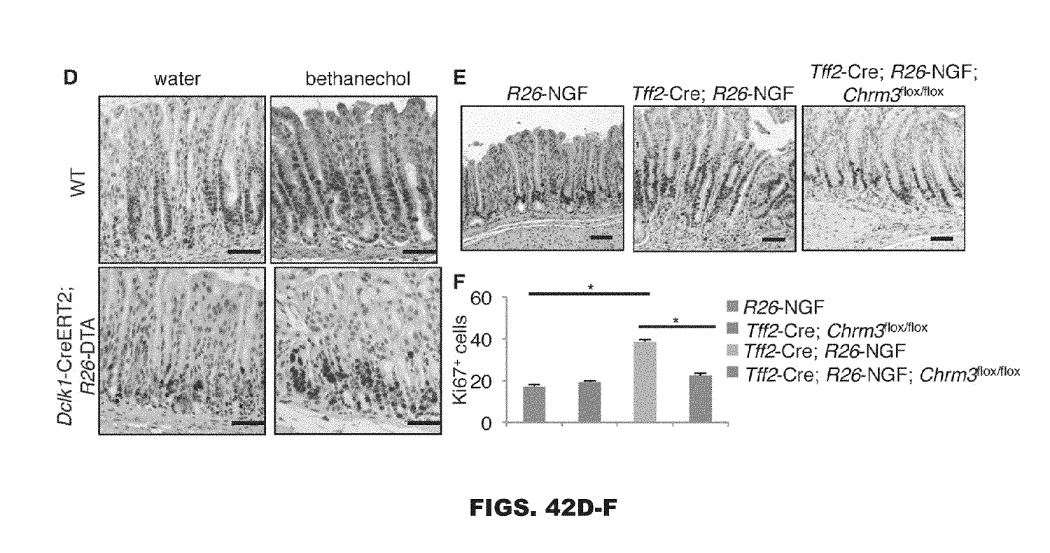

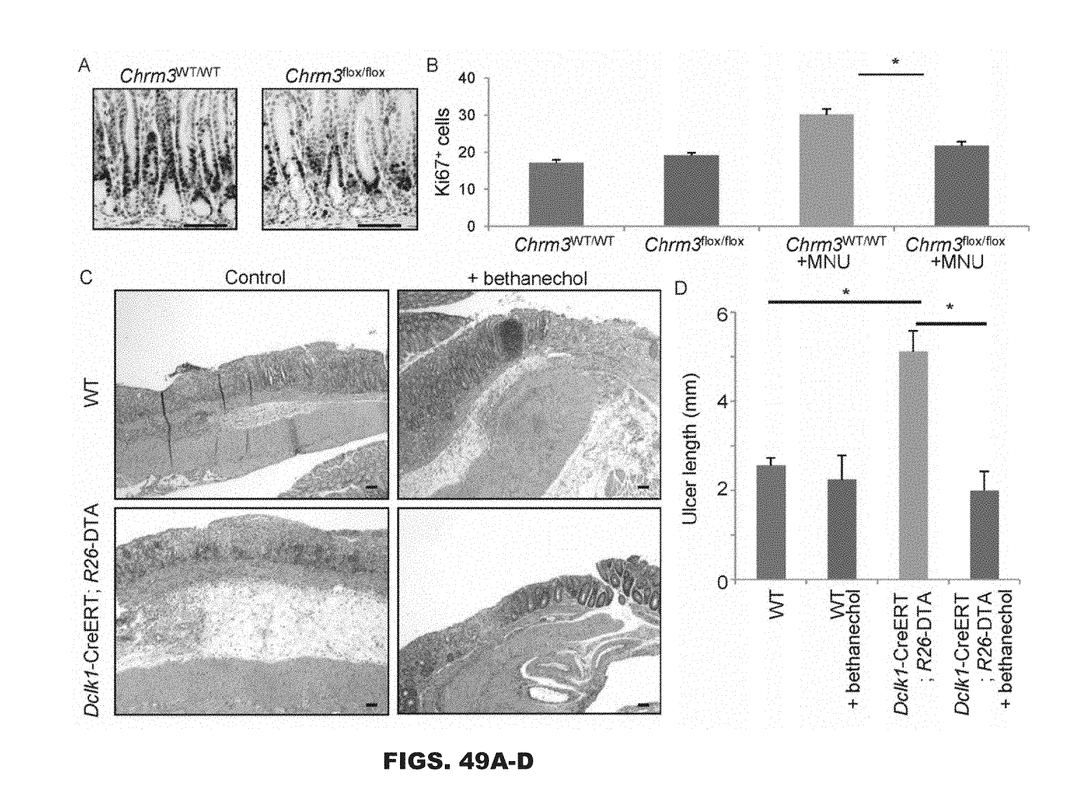

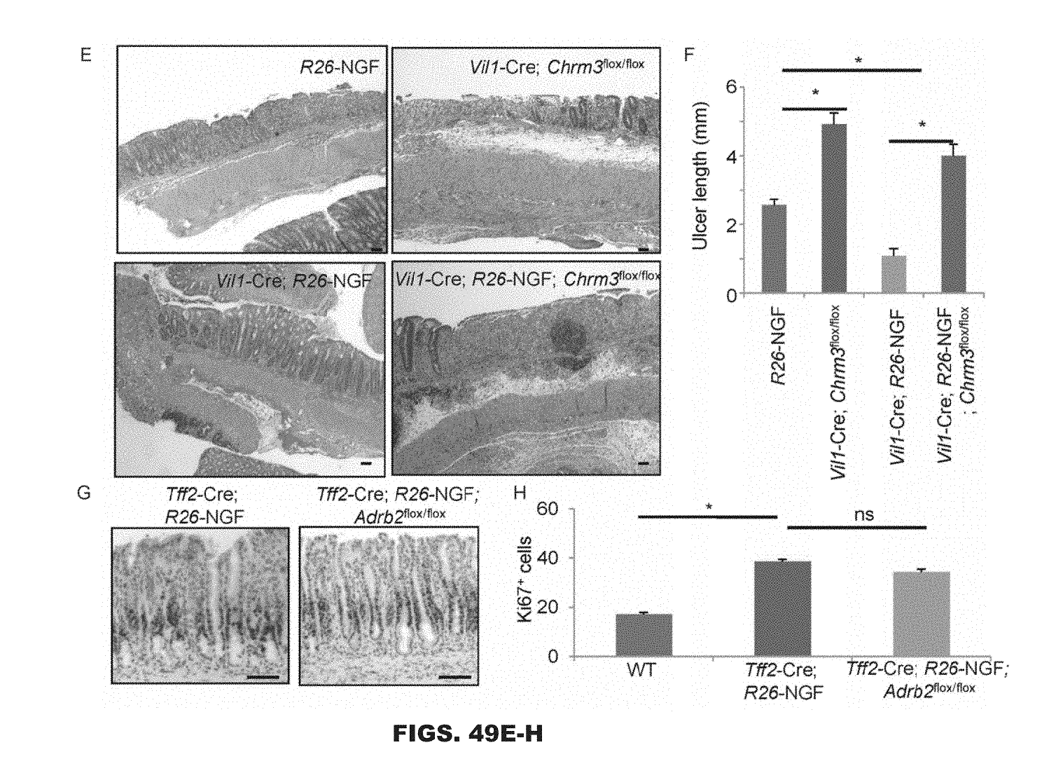

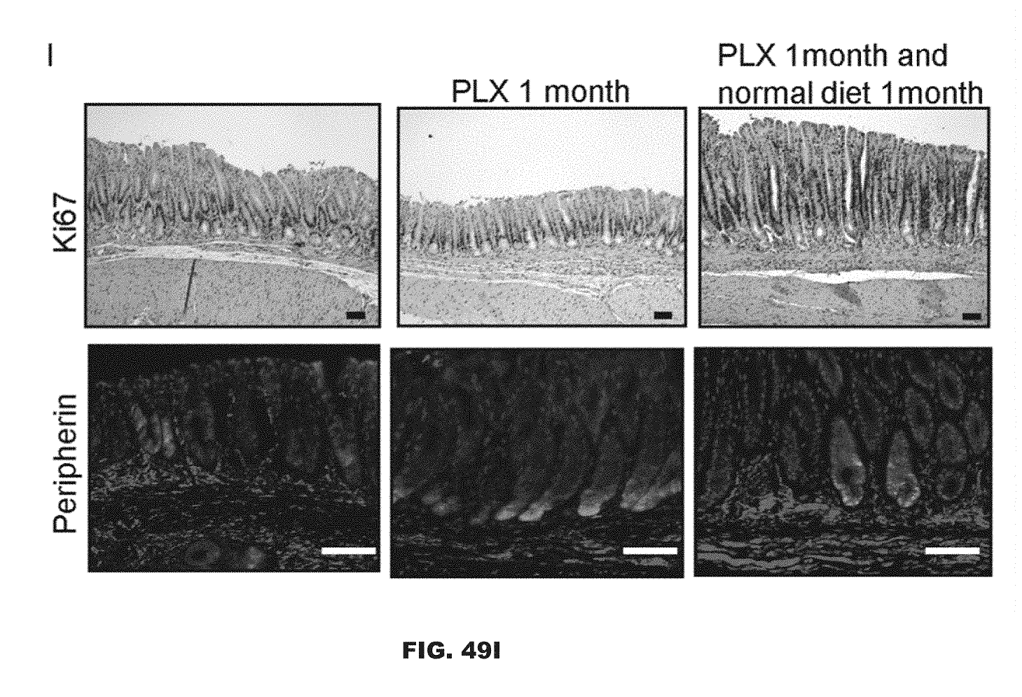

To test the role of muscarinic signaling in this process, Tff2-Cre; Chrm3^{flox/flox} mice were used to conditionally delete the Chrm3 gene in the gastric epithelium. Loss of epithelial M3R expression resulted in decreased proliferation following MNU-induced injury, while there were no significant changes in proliferation under normal conditions (FIGS. 49A and 49B). To examine the effect of M3R signaling in the gastric Lgr5⁺ population, Lgr5-CreERT; R26-Confetti mice with or without the Chrm3^{flox/flox} transgene were generated, and the clonal expansion of Lgr5+ stem cells during MNU treatment was traced. As reported previously (Leushacke et al., 2013), multiple Lgr5⁺ cells began to lineage trace each gland, with some glands showing all four of the different fluorescent reporters, but within 2 months most glands consolidated to a single color in a “neutral drift” manner. This single-color conversion in a gland was more frequently observed in MNU-treated mice than in non-treated mice, suggesting more rapid loss of progenitors or faster emergence of a dominant clone after MNU treatment (FIGS. 42A and 42B). However, knock out of Chrm3 in the Lgr5⁺ population suppressed single-color conversion, with the majority of glands in these mice demonstrating multi-color tracing or incomplete tracing even 2 months after tamoxifen/MNU treatment. Thus, M3R signaling may regulate gastric epithelial proliferation and stem cell division during mucosal regeneration.

It was previously reported that ablation of Dclk1⁺ cells leads to a decrease in epithelial proliferation in the intestine and colon (Westphalen et al., 2014). A similar decrease in gastric proliferation following Dclk1⁺ cell ablation in Dclk1-CreERT; R26-diphtheria toxin A (DTA) mice was confirmed (FIGS. 42C and 42D). In the Dclk1-CreERT; R26-DTA model, tuft cells were efficiently ablated after tamoxifen administration through cell-specific expression of DTA. However, the majority of labeled Dclk1⁺ nerves was not ablated and remained within the stroma, probably because of the requirement of high levels of Dclk1 expression for DTA-driven ablation. This provided the opportunity to examine the effect of specific ablation of tuft cells, as opposed to neurons, in these mice. The effects of a cholinergic agonist bethanechol following ablation of Dclk1⁺ tuft cells was tested, and administration of bethanechol significantly rescued the loss of proliferation by Dclk1⁻ tuft cell ablation, suggesting that the effect by tuft cell ablation in proliferation depends, at least in part, on the loss of their cholinergic signaling.

Dclk1⁺ cholinergic signaling also contributed to intestinal regeneration following DSS-colitis. While ablation of Dclk1 ⁺ cells worsened DSS-colitis (Westphalen et al., 2014), restoration of cholinergic signaling by bethanechol restored mucosal regeneration (FIGS. 49C and 49D). Furthermore, consistent with previous reports using systemic Chrm3 knockout mice (Hirota and McKay, 2006), conditional knock out of M3R in the colonic epithelium exacerbated the severity of DSS-colitis (FIGS. 49E and 49F). Intestinal overexpression of NGF in Vil1-Cre;R26-NGF mice with marked cholinergic innervation improved regeneration following DSS-induced injury, whereas simultaneous knock out of M3R blocked the NGF-mediated regenerative effects (FIGS. 49E and 49F). Taken together, these findings suggest that cholinergic signaling from tuft cells and nerves is critical for mucosal homeostasis and regeneration. Compared with control mice, Tff2-Cre; R26-NGF mice showed increased proliferation within the stomach (FIGS. 42E and 42F). The increased proliferation was abrogated by the concomitant knock out of Chrm3, but unaffected by the deletion of b2-adrenergic receptor (Adrb2), indicating that it was the cholinergic, rather than adrenergic, neurons that were the major drivers of epithelial proliferation in this setting (FIGS. 42E, 42F, 49G, and 49H). Treatment with the Trk inhibitor PLX for 1 month dramatically reduced the nerve density within the gastric mucosa, as well as the level of epithelial proliferation (FIG. 49I). However, the gastric epithelium returned to its baseline hyperproliferation following removal of the Trk inhibitor. This demonstrates that gastric ACh/NGF/Trk signaling is important for promoting both mucosal nerve fiber growth and epithelial proliferation.

Initiating the ACh-NGF Axis is Sufficient to Cause Gastric Cancer

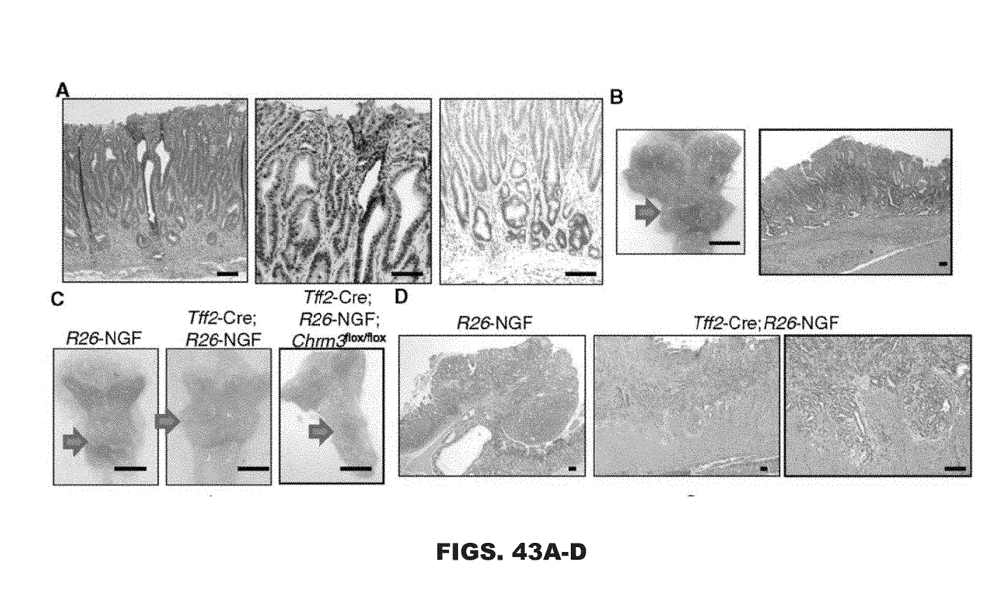

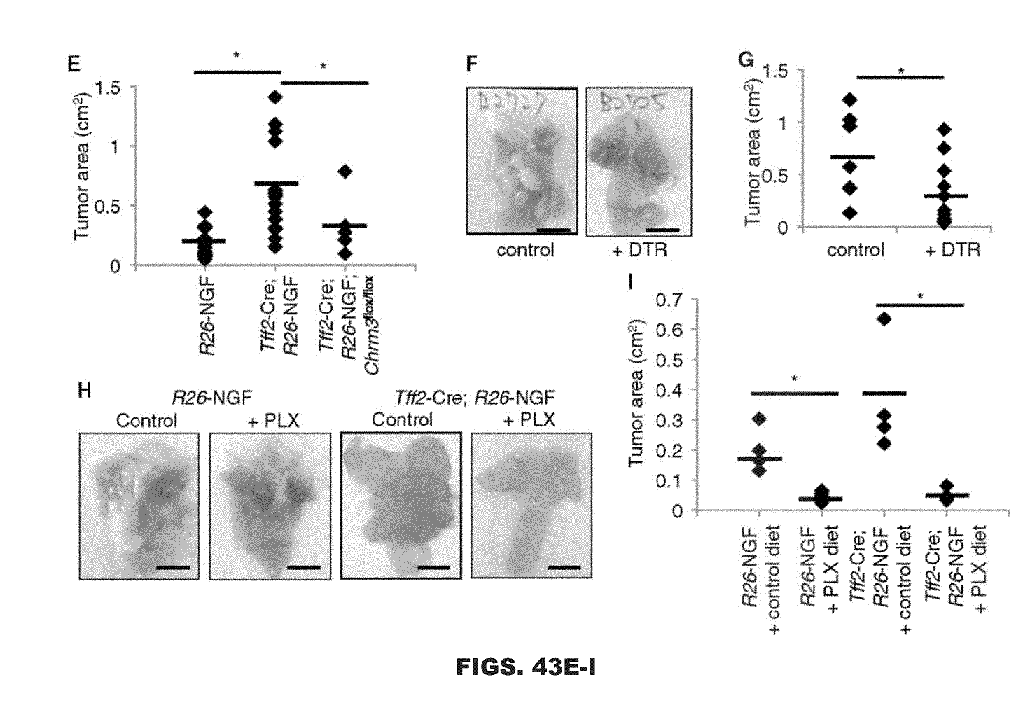

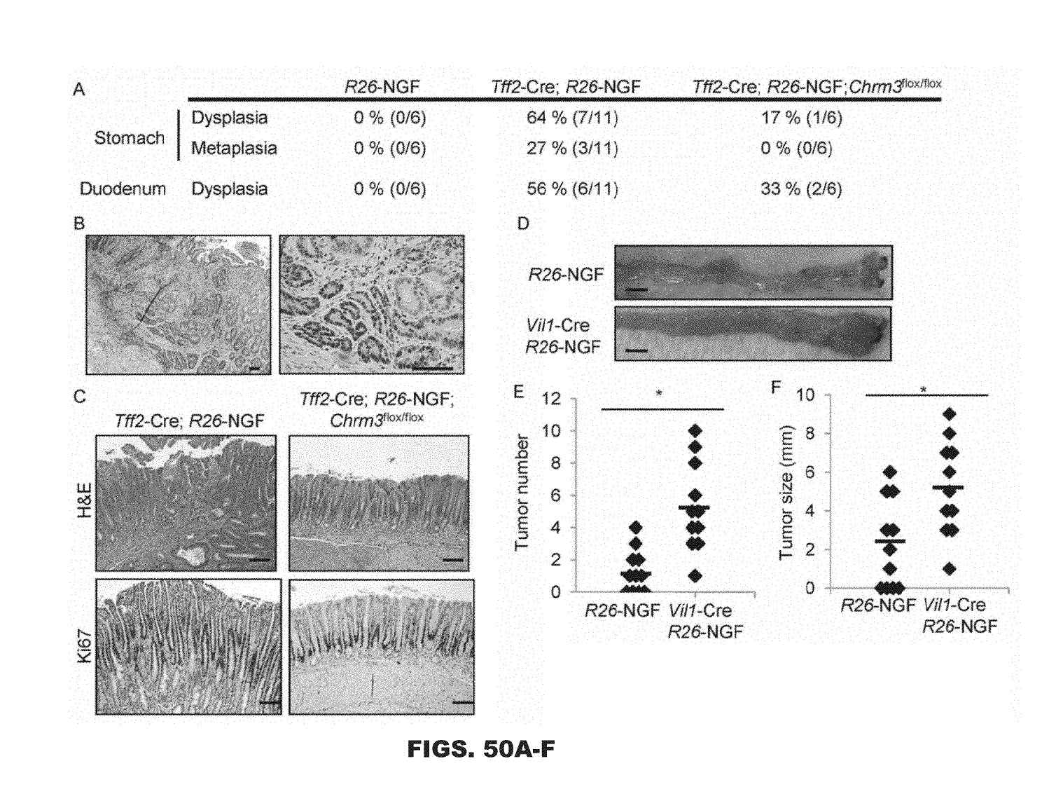

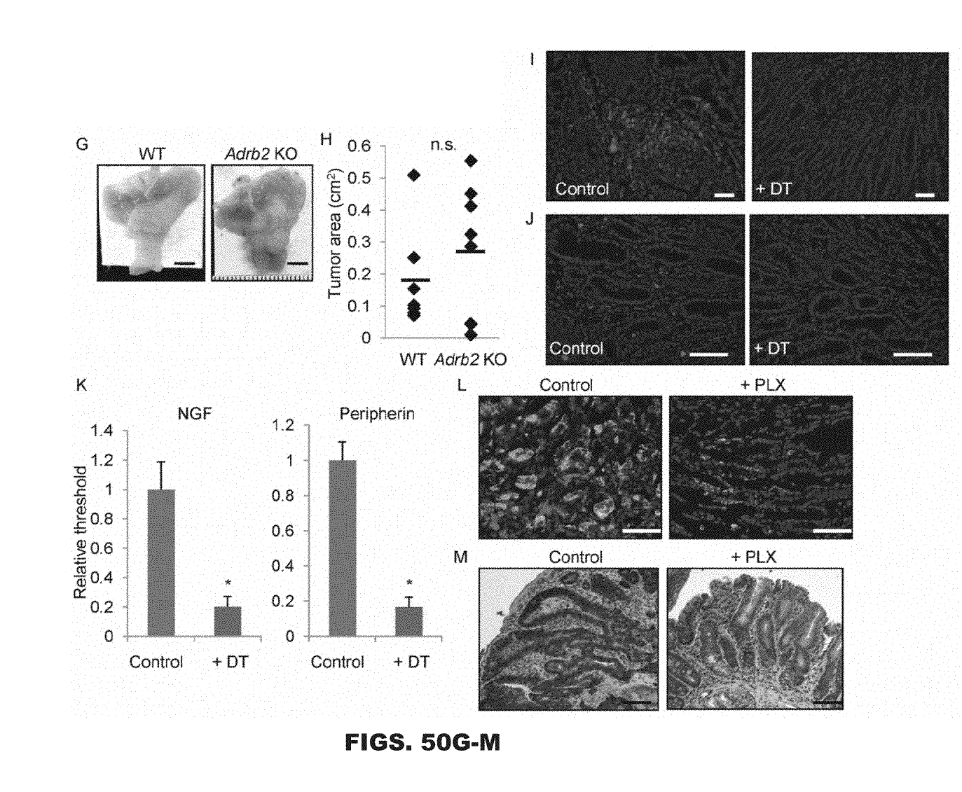

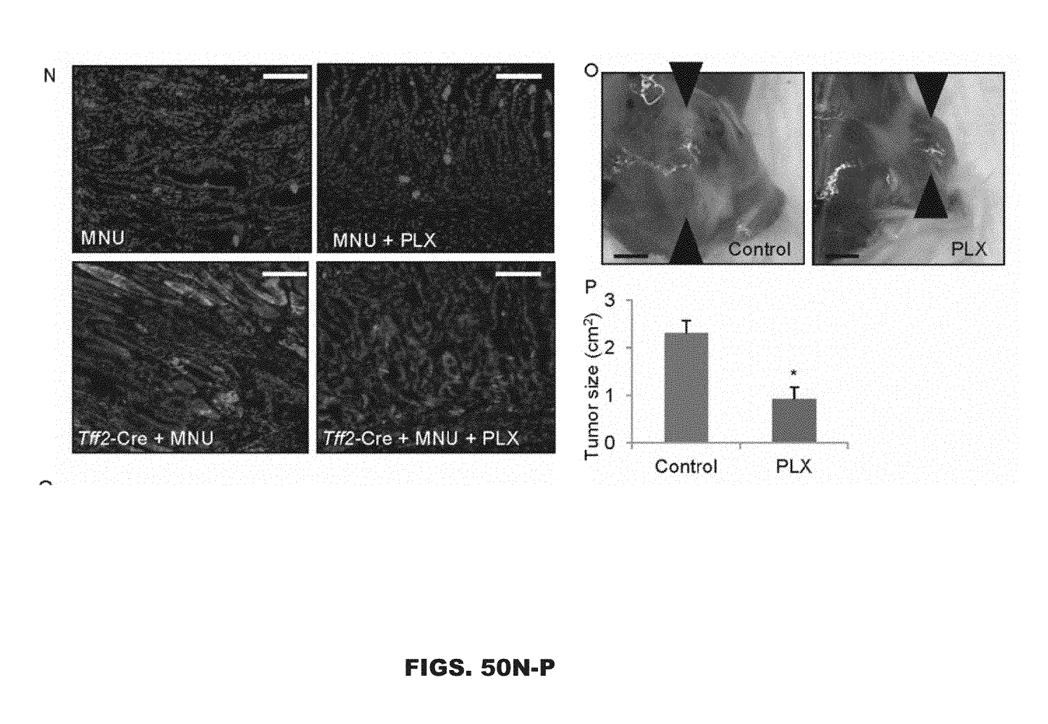

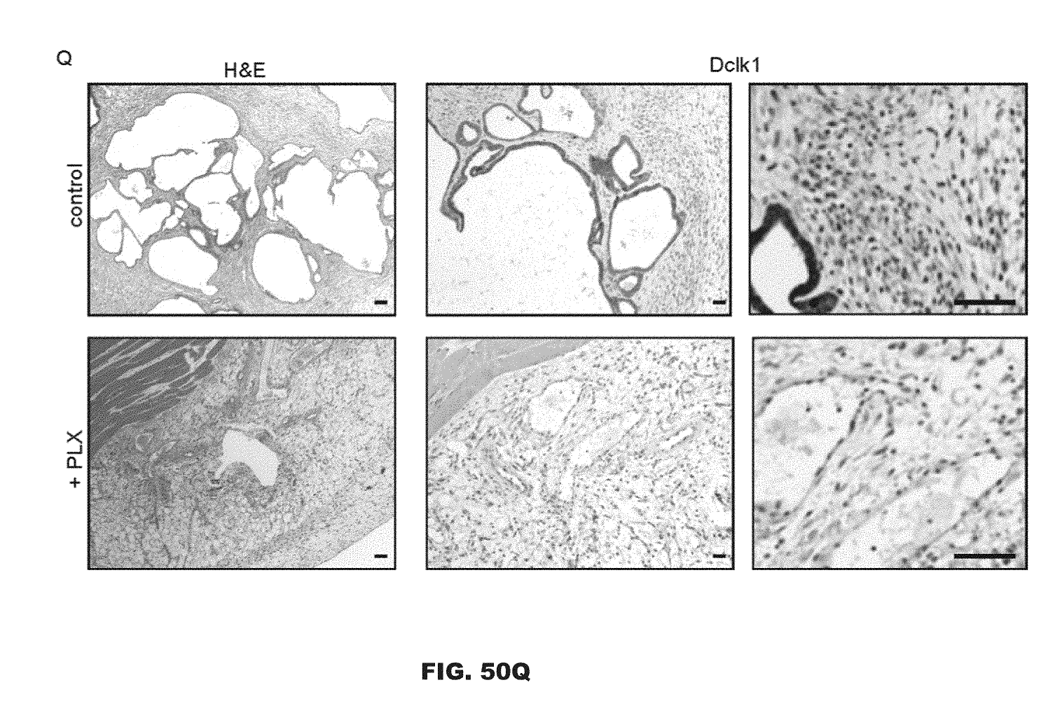

By 8 months of age, Tff2-Cre; R26-NGF mice developed spontaneous metaplasia and dysplasia in the stomach, with an expansion of dysplastic CD44⁺/ki67 ⁺ epithelial cells (FIGS. 43A and 50A). By 18 months, the mice developed large gastric tumors with intramucosal adenocarcinoma (FIG. 43B). Overexpression of NGF in colonic epithelium also induced dysplastic tumors in the rectum (FIG. 50B). These NGF-mediated effects in the stomach were attenuated by knock out of Chrm3 in gastric epithelium (FIGS. 50A and 50C). Overexpression of NGF in the Tff2-Cre; R26-NGF mice significantly accelerated tumor growth and invasion in response to MNU (FIGS. 43C-43E). The tumor-promoting effect by NGF was also evident in the AOM-DSS colon cancer model (FIGS. 50D-50F). Knock out of Chrm3 in the gastric epithelium blocked MNU-dependent tumor development in the setting of NGF overexpression (FIGS. 43C and 43E). By contrast, knock out of Adrb2 did not suppress MNU-dependent tumor development (FIGS. 50G and 50H), again establishing the role of cholinergic rather than adrenergic nerves in gastric carcinogenesis. Ablation of ACh-producing Dclk1+tuft cells in the MNU model by treatment of Dclk1-CreERT; R26-DTR mice with DT significantly inhibited tumor development, with reduction of NGF expression and innervation (FIGS. 43F, 43G, and 50I-50K). Treatment with the Trk inhibitor PLX prevented MNU tumor growth both in WT and NGF-overexpressing mice, and reduced peritumoral nerve density, CD44+ dysplastic cell expansion, and b-catenin nuclear translocation (FIGS. 43H, 431, and 50L-50N).

The effect of NGF inhibition was tested in an allograft tumor model (Li et al., 2014). Kras, Apc, and Tp53-mutated gastric glands were isolated from tamoxifen-treated Lgr5-CreERT; LSL-Kras^{G12D/+}; LSL-p53^{R172H/+}; Apc^{flox/flox} mice and tumor organoids cultured. These organoids were then implanted into immunodeficient NOD-SCID mice, and the mice were treated with a control or PLX7486-containing diet. After 3 weeks, tumor size was significantly reduced by Trk inhibition, and the expansion of nerves in the peritumoral site was inhibited (FIGS. 50O-50Q). Taken together, these experiments suggest a central role for NGF/Trk signaling, mediated through a cholinergic niche, in the initiation and progression of stomach cancer.

M3R Signaling Regulates Apc-Dependent Tumor Growth through YAP Activation

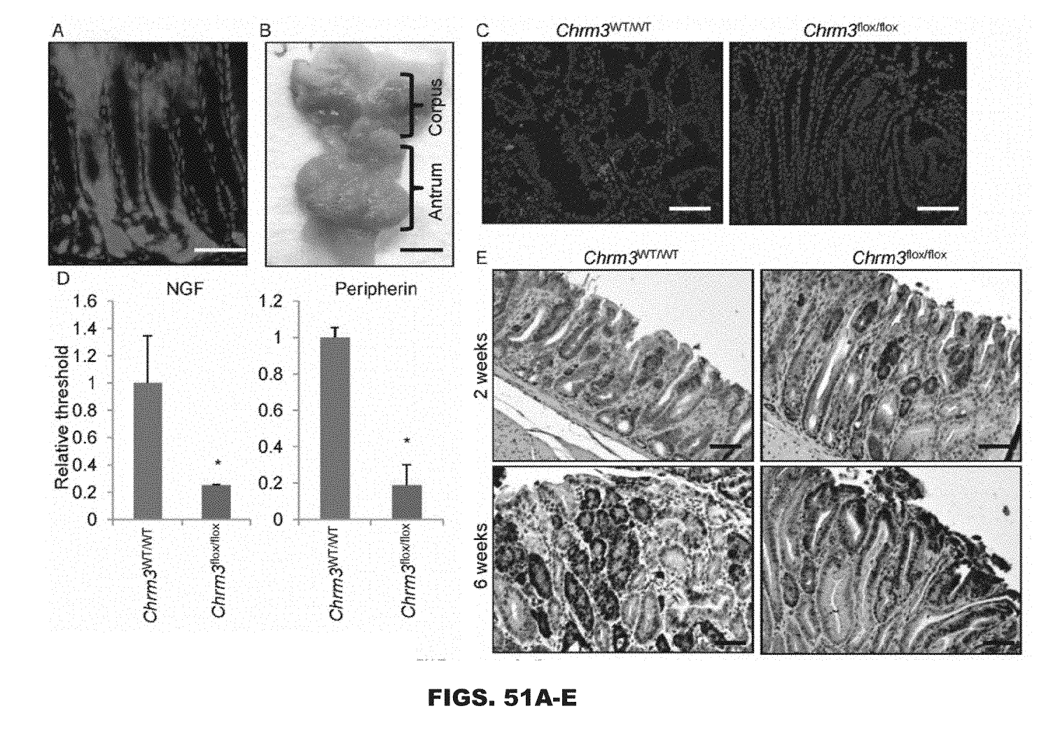

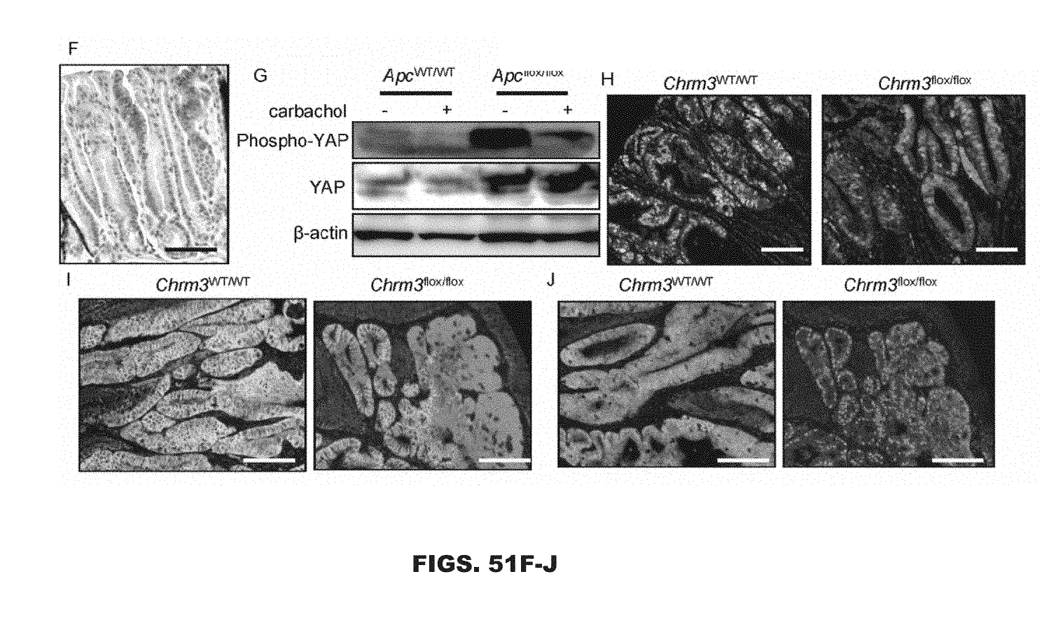

It was recently reported that Mist1+ stem cells in the oxyntic glands of the proximal stomach that express Bhlha 15 gene (also known as Mist1) can give rise to gastric cancer (Hayakawa et al., 2015a). In Mist1-CreERT; R26-Tomato mice, robust lineage tracing in the distal stomach was also found (FIG. 51A), suggesting the fair and broad expression of Mist 1 in the gastric antrum. Interestingly, loss of Apc gene in the Mist1 lineage was not sufficient to create tumors in the proximal corpus, as reported previously (Hayakawa et al., 2015a, 2016); however, Apc deletion in the Mist1 lineage did induce rapid macroscopic tumors in the gastric antrum (FIG. 51B).

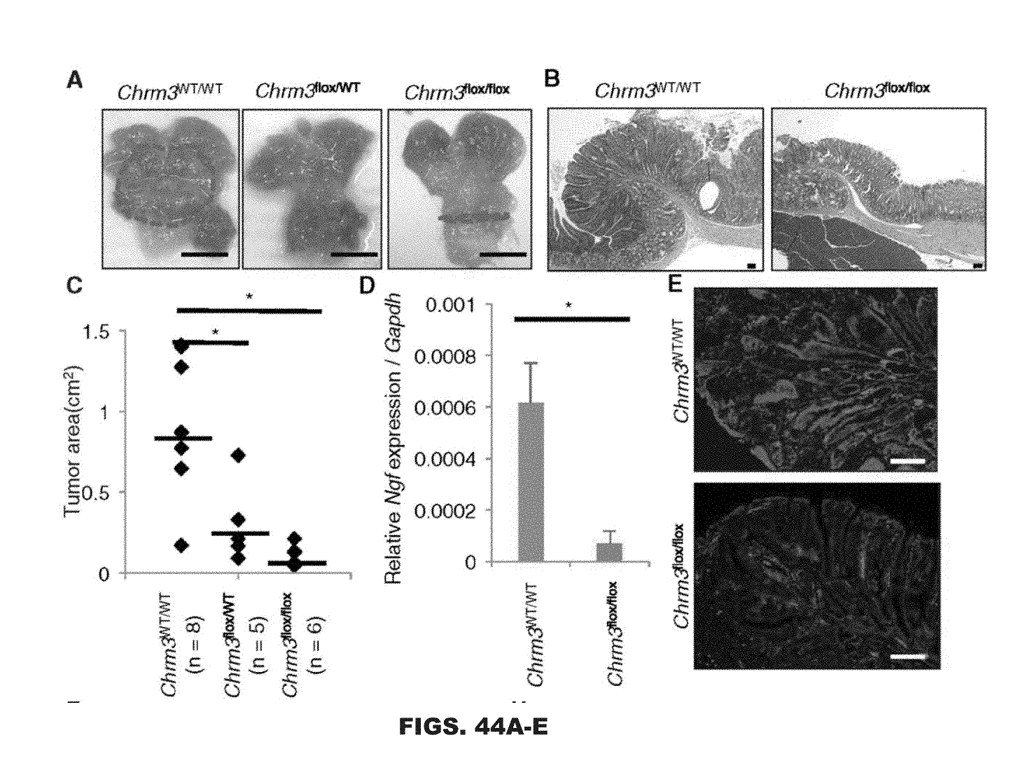

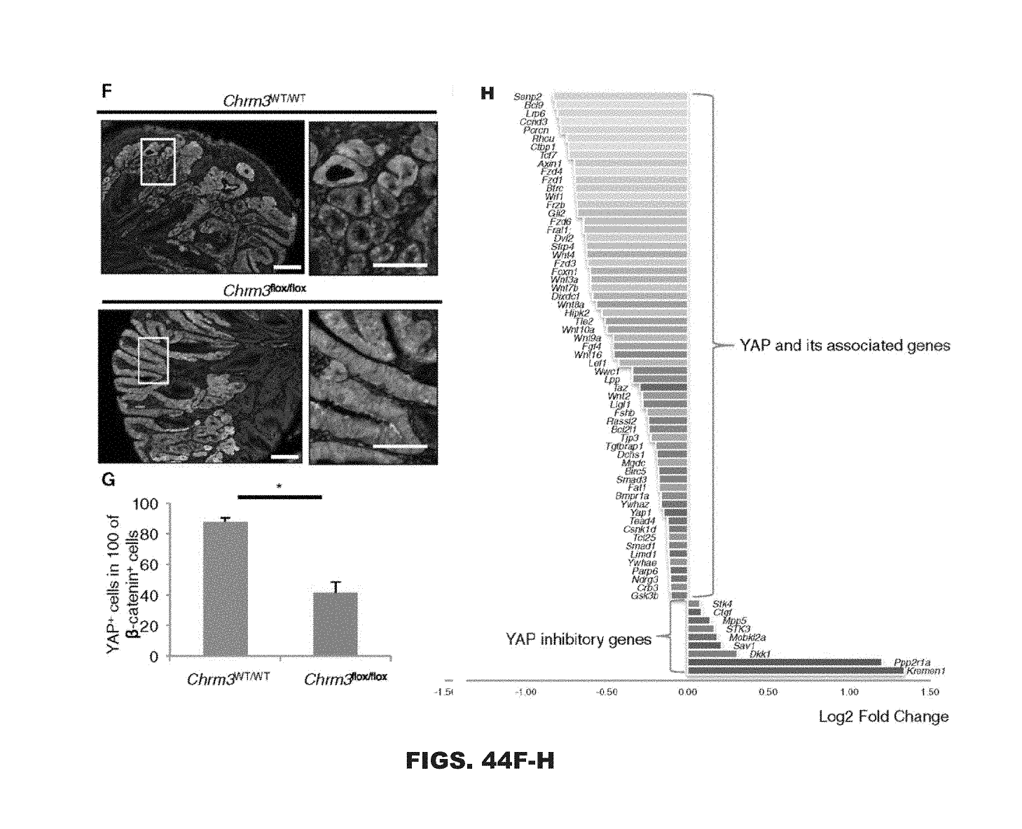

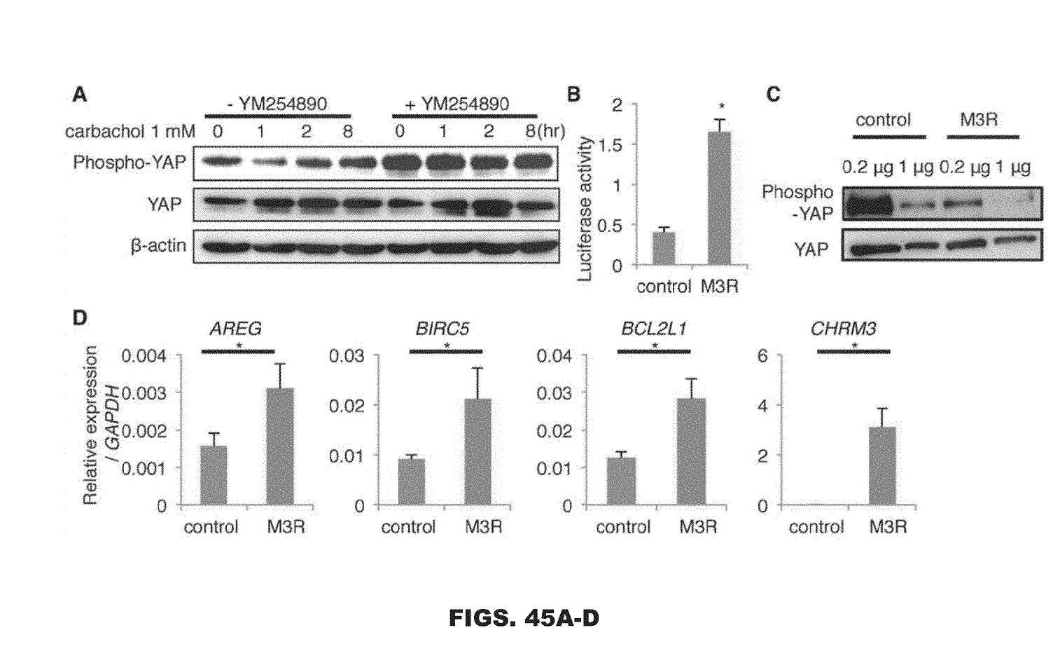

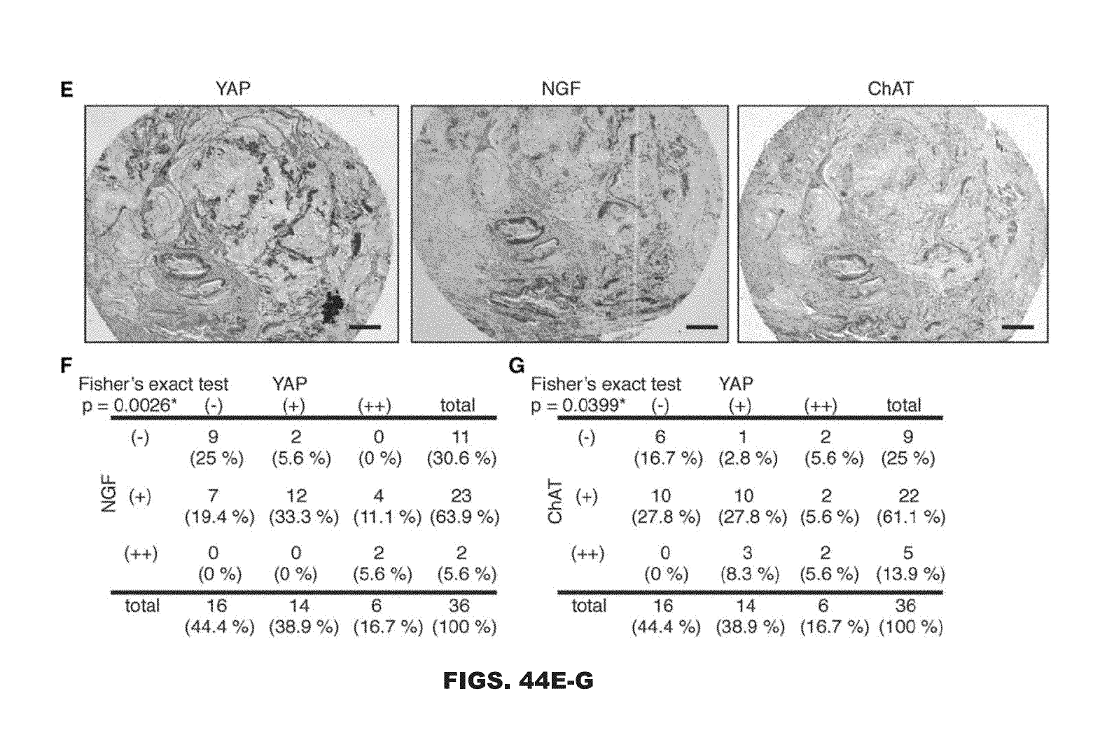

Using this mouse model of antral gastric tumor, Mist1-CreERT; Apc^{flox/flox}; Chrm3^{flox/flox} mice were generated to explore the role of M3R signaling in Apc/b-catenin-dependent tumor growth. In keeping with the role of the cholinergic signaling described above, it was also found that a significant reduction in tumor burden with loss of just one copy of Chrm3 gene, and hemizygous Chrm3-floxed mice showed an almost complete absence of macroscopic tumor development (FIGS. 44A-44C). NGF expression and nerve density were significantly decreased in Apc and Chrm3 double knockout mice, supporting the role of cholinergic signaling on NGF-mediated innervation (FIGS. 44D, 44E, 51C, and 51D). Nevertheless, immunostaining showed strong b-catenin nuclear accumulation both in Chrm3-WT and Chrm3-null stomachs (FIG. 51E), suggesting that knock out of M3R suppresses tumor growth downstream of b-catenin nuclear translocation, possibly by inhibiting TCF transcriptional activity.

YAP modulates Wnt/b-catenin signaling as a transcriptional co-activator, and is required for intestinal tumor growth following the loss of Apc (Azzolin et al., 2014; Rosenbluh et al., 2012). While YAP is minimally expressed in normal gastric epithelium or cultured organoids, strong YAP upregulation is observed in dysplastic tissues or organoids after Apc deletion (FIGS. 44F, 51F, and 51G). However, YAP was rarely upregulated within the Chrm3-null gastric dysplasia, even though they showed strong b-catenin expression (FIGS. 44F and 44G). A gene downstream of YAP, BCL2L1, was also downregulated in the Chrm3-null gastric dysplasia compared with the Chrm3-WT mice (FIG. 51H). The Wnt target genes, Sox9 and CD44, were prominently expressed in Chrm3-WT, nuclear b-catenin¹ dysplastic cells, but not in the Chrm3-null, b-catenin⁺ cells (FIGS. 51I and 51J). Furthermore, previous microarray data (GEO: GSE30295) was revisited which compared gene expression between gastric tumors isolated from the vagotomized anterior stomach and the non-vagotomized posterior stomach in a hypergastrinemic mouse model (Zhao et al., 2014). Downregulation of YAP-associated genes along with upregulation of YAP inhibitory genes was found in the vagotomized anterior stomach, suggesting that the inhibition of ACh signaling by vagotomy can block YAP activity in this model (FIG. 44H). Thus, M3R signaling may regulate tumor growth in mice by controlling YAP activity.

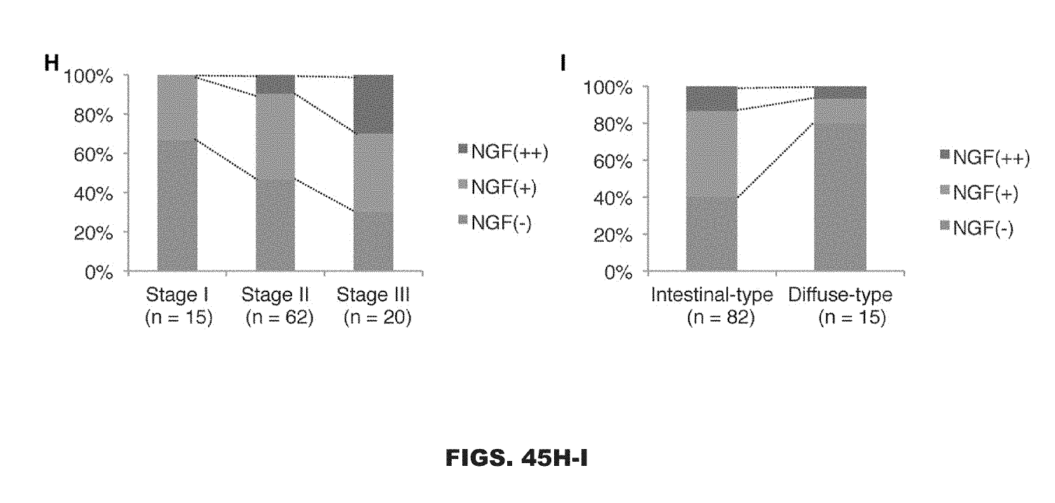

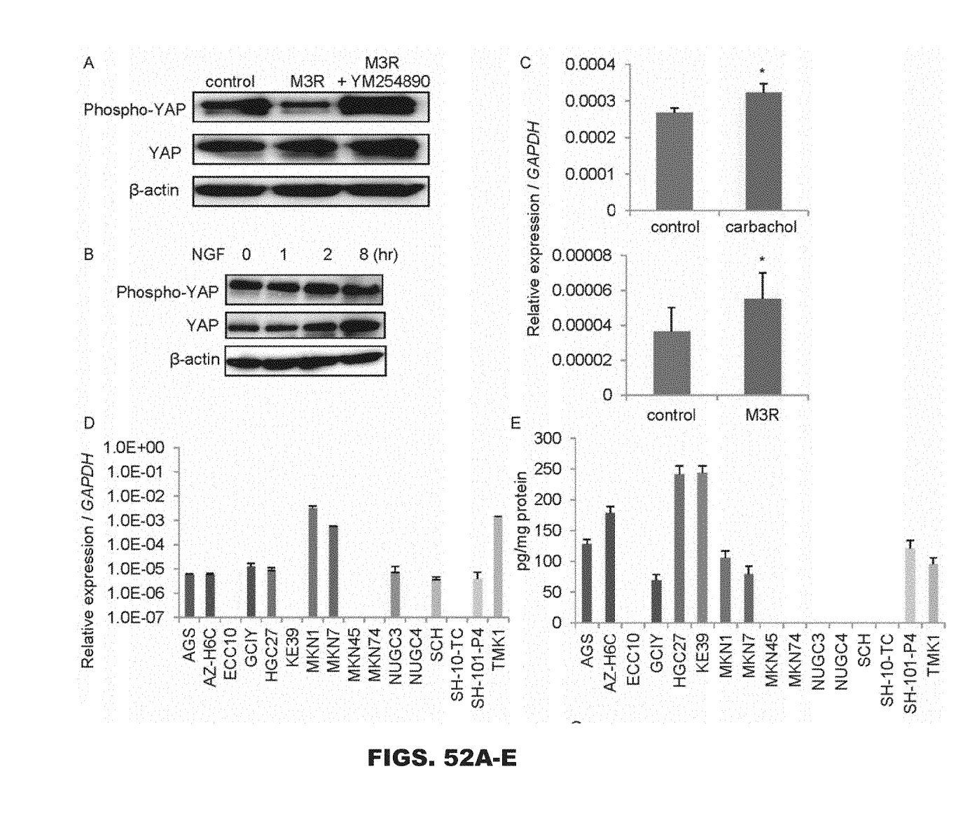

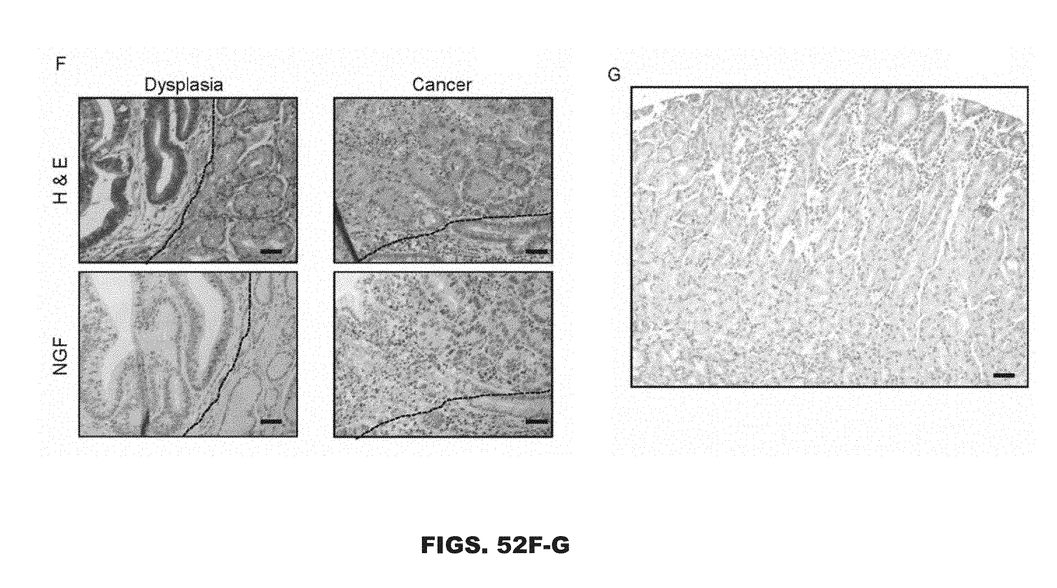

The M3R, a G-protein-coupled receptor (GPCR), selectively couples to G-proteins of the Gq/11 family, and it has been suggested that GPCRs regulate YAP activation by controlling large tumor suppressor kinase activity (Feng et al., 2014; Yu et al., 2012, 2014). In the TMK1 gastric cancer cell line which expresses the M3R (Kodaira et al., 1999), treatment with carbachol reduced the level of phosphorylated YAP, with no significant changes in the levels of total YAP (FIG. 45A). Similarly, carbachol treatment in Apc-deleted gastric organoids reduced the level of phosphorylated YAP (FIG. 51G). However, treatment with a Gq/11-specific inhibitor YM254890 blocked the changes of YAP phosphorylation in TMK1 cells after carbachol stimulation, suggesting that cholinergic stimuli dephosphorylates YAP indeed through a Gq/11 family protein. Next a human M3R gene-expressing construct was transfected into the M3R-negative AGS cell line. Overexpression of M3R decreased the level of phosphorylated YAP in a Gq-dependent manner, and activated the transcriptional activity of YAP in a luciferase assay (Dupont et al., 2011) (FIGS. 45B, 45C, and 52A). YAP target genes including AREG, BIRC5, and BCL2L1 were significantly upregulated by M3R overexpression (FIG. 45D). Consistent with the findings in the mouse models, carbachol treatment or M3R overexpression significantly upregulated NGF expression in human cancer cells, although NGF treatment did not cause YAP activation contrary to carbachol (FIGS. 52B and 52C).

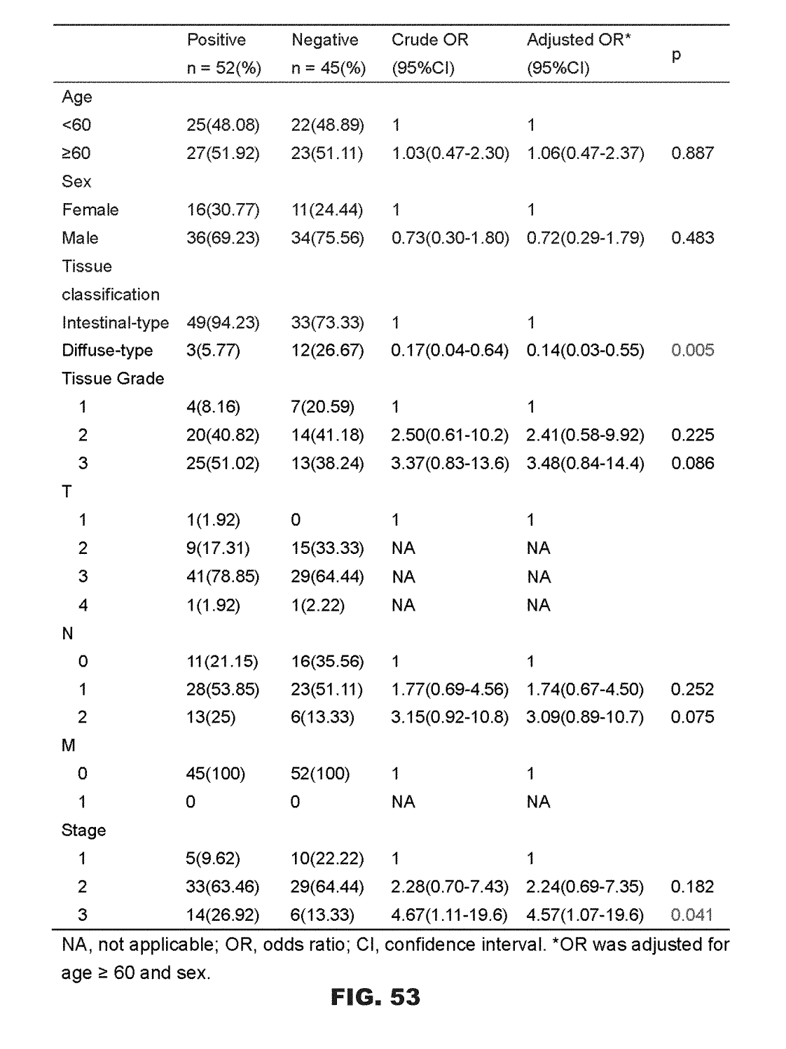

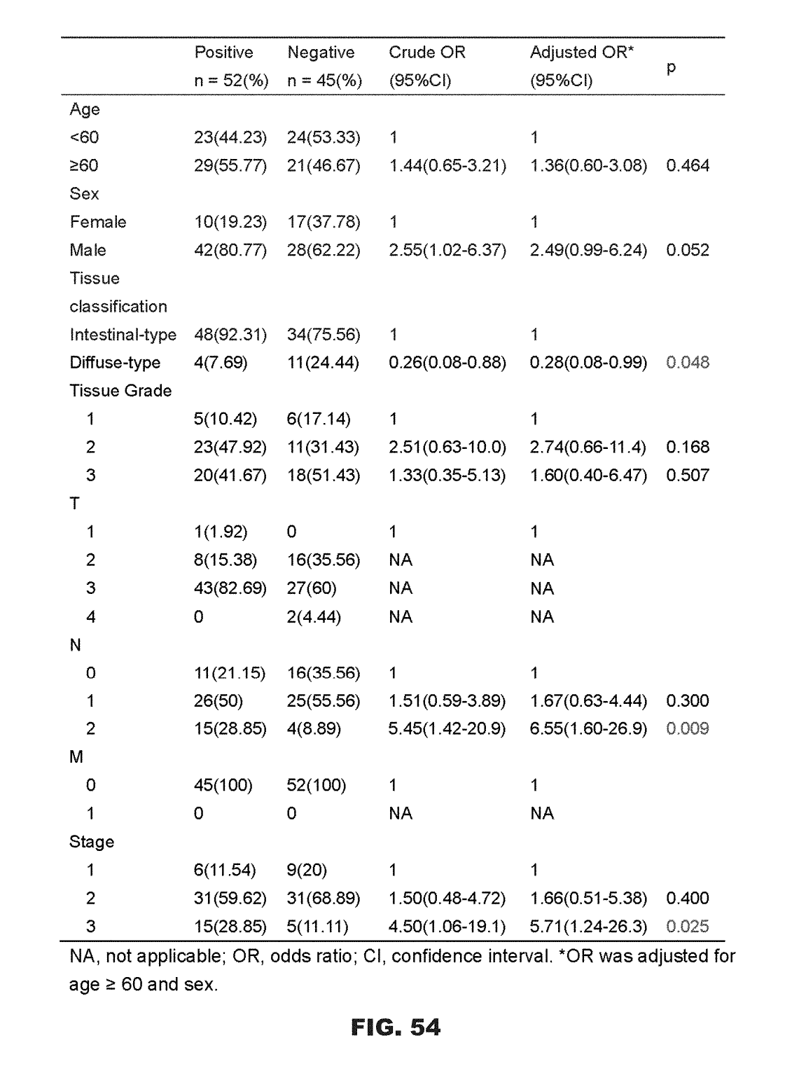

NGF expression levels were investigated in 16 gastric cancer cell lines (FIGS. 52D and 52E), and confirmed NGF mRNA expression in 10 lines (62.5%), and NGF protein expression in 9 lines (56.3%). Staining of 36 human gastric cancer samples with NGF antibody demonstrated that NGF was moderately expressed in 63.9% of cancer patients and strongly expressed in 5.6% patients, while non-dysplastic stomach does not express NGF or YAP (FIGS. 45E-45G, 52F, and 52G). Importantly, there was a significant correlation between YAP immunoreactivity and NGF or ChAT immunoreactivity. In addition, we evaluated NGF and YAP expression in an additional set of 97 human gastric cancer samples, and correlated expression with clinical and histopathological data. In this additional cohort, NGF was expressed in 53.6% of cancer cases. NGF expression was significantly associated with a higher cancer stage (adjusted odds ratio [AOR] of 4.57), and expression was more evident in intestinal-type cancers than in diffuse-type cancers (FIGS. 45H and 45I and 53). YAP expression was also significantly associated with cancer stage (AOR of 5.71), as well as an increased risk of lymphoid node metastasis (AOR of 6.55) (FIG. 54). Taken together, these results support the significance of the NGF-ACh-YAP axis in human gastric cancers, particularly in advanced, intestinal-type cancers.

Discussion

It was found that Dclk1 and ChAT are co-expressed in both tuft cells and nerves within the stomach and intestine, and that both cholinergic sources expand at discrete times during carcinogenesis. Tuft cells are dramatically expanded in early carcinogenesis, particularly in inflammation-associated cancer models. Previous studies suggested that tuft cells could potentially influence carcinogenesis through production of inflammatory mediators (Bailey et al., 2014; Okumura et al., 2010; Quante et al., 2012), but here it is shown that tuft cells are also a local source of ACh that contributes to early cancer growth and remodeling of the peritumoral neural microenvironment.

It is proposed that the ACh-NGF-positive feedback loop is the basis for the abnormal innervation observed in the tumor microenvironment. Previous studies have reported that NGF can be induced by cholinergic stimuli (da Penha Berzaghi et al., 1993; Lapchak et al., 1993; Mahmoud et al., 2015), and in turn NGF can promote cholinergic nerve growth (Collins, 1984; Collins and Dawson, 1983; Kniewallner et al., 2014), consistent with the results described herein. While the major receptor for NGF is TrkA, another neurotrophin receptor p75NTR can bind to NGF and pro-NGF (Howard et al., 2013). Given the significant effects by the Trk inhibitor, it appears that Trk is the primary receptor within the ACh-NGF axis. Many clinical studies have investigated the efficacy of Trk inhibitors in cancers with mitogenic Trk-fused mutations (Vaishnavi et al., 2015). Furthermore, anti-NGF antibody has been used in several clinical studies for testing its effect on pain management in osteoarthritis patients (Bannwarth and Kostine, 2014). The preclinical results described herein suggest that Trk inhibitors and anti-NGF antibody can be effective for the therapy for stomach cancer by targeting the ACh-NGF axis.

The muscarinic ACh receptors are classified into five distinct subtypes; gastric epithelial cells primarily express M3R, and also low level of muscarinic ACh receptor-1 (M1R) and receptor-5 (M5R) (Aihara et al., 2005; Zhao et al., 2014). The activation of M3R leads to a variety of biochemical and electrophysiological responses, and the resulting physiological effects may depend on the cell types. It has been suggested that M3R can activate mitogen-activated protein kinase (Kodaira et al., 1999), Akt (Song et al., 2007), or RhoA (Belo et al., 2011) and contribute to tumor growth in various cancers. It has been reported that M3R activates the Wnt pathway, and the results described herein suggest that M3R-mediated Wnt activation may be through YAP, a downstream target of M3R.

YAP, a downstream effector of the Hippo pathway, regulates various cellular functions such as proliferation, survival, stemness, or pluripotency. YAP is upregulated and activated by loss of Apc, and in turn YAP activation is required for b-catenin-dependent cancer growth (Azzolin et al., 2014; Cai et al., 2015; Rosenbluh et al., 2012). Accordingly, YAP controls tissue regeneration and tumorigenesis in various organs including stomach, by activating tissue stem cells (Gregorieff et al., 2015; Imajo et al., 2015; Jiao et al., 2014). GPCRs that activate G12/13, Gq/11, or Gi/o, can activate YAP by repressing YAP phosphorylation, while GPCRs that mainly activate Gs signaling such as Adrb2, are able to induce YAP phosphorylation and subsequent YAP inhibition (Yu et al., 2012). The results described herein suggest that the M3R can activate YAP signaling in a manner similar to other Gq/11 family receptors. Thus, destruction of the Apc complex is only able to induce full activation of Wnt targets and aggressive tumor development when there is sufficient, permissive cholinergic signaling through the M3R. Although it seems that M3R regulates YAP activity predominantly through its dephosphorylation, the involvement of M3R in YAP upregulation during initial malignant transformation remains uncertain, and thus needs to be elucidated in future studies.

In summary, the results described here have elucidated the machinery of nerve-epithelial interaction within the stomach, including the source of ACh, the cause of abnormal innervations in cancer, and the regulation of the Wnt and YAP pathways by M3R signaling, constituting what is termed the ACh-NGF-YAP axis. Inhibition of ACh, M3R, or NGF can be a therapeutic strategies in the treatment of gastrointestinal cancers.

Experimental Procedures

Mice

Mist1-CreERT2 (Shi et al., 2009), Dclk1-CreERT (Westphalen et al., 2014), Tff2-Cre (Dubeykovskaya et al., 2016), Chrm3^{flox}(Gautam et al., 2006), Chrm3 knockout (Aihara et al., 2003), Adrb2^{flox}(Hinoi et al., 2008), and Nes-GFP (Mignone et al., 2004) mice were described previously. Apc^{flox} mice were obtained from the National Cancer Institute. Lgr5-CreERT-IRES-EGFP, Vil1-Cre, Chat-GFP, NOD-SCID, Adrb2 knockout, R26-mTmG, R26-TdTomato, R26-Confetti, R26-DTR, and R26-DTA mice were purchased from the Jackson Laboratory. R26-LSL-Ngf-IRES-EGFP mice were generated as follows: the R26-LSL-Ngf-IRES-GFP alleles were generated by inserting CAG-loxP-STOP-LoxP-Ngf-IRES-GFP-poly(A) cassettes into a Rosa-acceptor targeting plasmid (CTV plasmid, a gift from Dr. Klaus Rajewsky [Addgene no. 15912]). Mouse lines where generated by homologous recombination in KV1 (129S6/SvEvTac x C57BL/6J) embryonic stem cells followed by injection into C57BL/6J blastocysts. Chimeras were bred for germline transmission. Mice were backcrossed to C57BL/6J background for at least six generations. Cre recombinase was activated in CreERT mouse lines by oral administration of TAM (3 mg/0.2 mL corn oil). All animal studies and procedures were approved by ethics committees. All protocols using human materials were approved by ethics committees, and written informed consent was obtained from all patients.

Treatment

MNU (Sigma) were prepared as described previously, and mice (8-week-old) were given drinking water containing 240 ppm MNU on alternate weeks for a total of 10 weeks (total exposure of 5 weeks) (Hayakawa et al., 2015b). For tumor analysis, mice were analyzed 36-52 weeks after the beginning of MNU, as indicated. For Dclk1+ cell ablation in Dclk1-CreERT; R26-DTR mice, tamoxifen and DT (10 mg/kg) were administered once a week. PLX-7486, provided from Plexxicon, has been used in Phase I clinical studies and detailed information is available on the NCI drug dictionary:

www.cancer.gov/publications/dictionaries/cancer-drug?cdrid=747694. PLX-7486 was mixed into the AIN-76A mouse chow (100 mg/kg), and fed for the indicated periods. The control group was given AIN-76A chow without PLX-7486. Bethanechol was dissolved in drinking water at a concentration of 800 mg/L. DSS was dissolved in drinking water at 3% and given for 5 days. Vagotomy was performed as described previously (Zhao et al., 2014).

Statistical Analysis

The differences between the means were compared using the Student's t-test (two-tailed). One-way ANOVA with post hoc test was performed for multiple comparisons. Fisher's exact test was used to determine if there are nonrandom associations between two categorical variables. In FIGS. 53 and 54, a logistic regression analysis was conducted to evaluate the odds ratios as an estimate of whether NGF and YAP expression (“Positive” includes [+] and [++] cases) was associated with each parameters. p values of <0.05 were considered to indicate statistical significance. All statistical analyses were performed using the JMP (v.11) and the SAS (v.9.4) software.

Accession Numbers

Microarray data which compared gene expression between gastric tumors isolated from the vagotomized anterior stomach and the non-vagotomized posterior stomach in hypergastrinemic mice were deposited in GEO database (GEO: GSE30295).

Gland Isolation and in vitro Culture System

The harvested mouse stomachs were opened longitudinally and washed with cold PBS. The tissue was chopped into approximately 5 mm pieces, washed with cold PBS, and incubated in 8 mM EDTA in PBS for 60 minutes on ice. The tissue fragments were suspended vigorously in cold 10% FBS using a 10-mL pipette, yielding supernatants enriched in crypts. Gland fractions were centrifuged at 900 rpm for 6 minutes at 4° C. and diluted with advanced DMEM/F12 (Invitrogen) containing B27, N2, 1 μM n-Acetylcysteine, 10 mM HEPES, penicillin/streptomycin, and Glutamax (all from Invitrogen). Samples were passed through 100 μm filters (BD Biosciences) and centrifuged at 720 rpm for 5 minutes at 4° C., and single cells were discarded. Glands were embedded in Matrigel (BD Biosciences) and 500 glands/well were seeded in a pre-warmed 24-well plate. After the Matrigel solidified, it was overlaid with advanced DMEM/F12 medium containing 50 ng/mL EGF (Invitrogen), 100 ng/mL Noggin (Peprotech), 1 μg/mL R-spondin 1, and 10 μg/mL Wnt3a. Growth factors were added every other day, and all medium contents were changed twice a week. For the isolation of Dclk1+ cells, stomach was chopped into approximately 5 mm pieces, washed with cold HBSS, and incubated in 0.1 mg/ml dispase and 3.5 mg/ml collagenase 4 in HBSS for 30 minutes at 37° C. The tissue fragments were suspended vigorously in 10% FBS using a 10 mL pipette, and the supernatant was centrifuged at 1300 rpm for 5 minutes, resuspended in 5 ml of 40% Percoll, and then centrifuged at 2000 g for 10 minutes, yielding the lymphocytes or endothelial cell fraction. Viable single cells were gated by forward scatter, side scatter and a pulse-width parameter, and propidium iodide-negative staining. For staining, cell suspensions were incubated with conjugated monoclonal antibodies against Epcam (purchased from BioLegend). Sorted cells were collected, pelleted and embedded in extracellular matrix, followed by seeding in a 48-well plate (100-3000 singlets per well). The images of gastric organoids were acquired using fluorescent microscopy (Nikon, TE2000-U) and two-photon microscopy (Nikon, A1RMP).

Immunohistochemistry

Immunohistochemistry and immunofluorescence were performed as described previously (Hayakawa et al., 2015). The primary antibodies used included the following: Ki67 (1:200, Abcam), beta-catenin (1:500, BD laboratories), GFP (1:400, Invitrogen or Abcam), peripherin (1:1000, Millipore), Dclk1 (1:200, Abcam), GAP43 (1:1000, Millipore), TH (1:1000, Millipore), VachT (1:200, Phoenix Pharmaceuticals), CD44 (1:200, BD laboratories), YAP (1:200, Cell Signaling), Sox9 (1:200, Millipore), S100B (1:1000, DAKO), GFAP (1:1000, DAKO), NGF (1:500, Santa Cruz or Abcam), PGP9.5 (1:1000, Abcam), HuC/D (1:1000, Thermofisher), CD31 (1:1000, DAKO), αSMA (1:200, Abcam), BrdU (1:200, DAKO), BCL2L1, (1:200, Bioss), and ChAT (1:500, a gift from Dr. Michael Schemann, Technische Universität München). In situ hybridization was performed on paraffin-embedded sections using the RNAScope 2.0 kit (Advanced Cell Diagnostics). Human gastric cancer samples were provided by Dr. Helen Remotti (Columbia University) and Dr. Hiroyuki Tomita, and obtained from Abcam tissue array.

Quantitative RT-PCR (qRT-PCR)

Total RNA was extracted from whole stomach samples from each animal using TRIzol reagent (Invitrogen) or the RNAqueous-micro kit (Ambion) and subjected to first-strand complementary DNA synthesis using the Superscript III cDNA Amplification System (Invitrogen) following the manufacturer's instructions. qRT-PCR was performed using a three-step method, an ABI 7300 system and SYBR green (Roche). The qRT-PCR primer sequences are available upon request. The results were expressed as the copy number of each gene relative to that of Gapdh or GAPDH.

Western Blotting

Protein lysates were prepared from cells, separated by sodium dodecyl sulfate-polyacrylamide gel electrophoresis (SDS-PAGE), transferred to polyvinylidene difluoride membranes (Millipore). The membrane was probed with primary antibodies (phospho-YAP (Cell Signaling, 1:1000), YAP (Cell Signaling, 1:1000, and β-actin (Sigma, 1:5000) antibodies), and then incubated with the secondary antibody. Immunocomplexes were detected using the enhanced chemiluminescence system (Amersham Biosciences).

Cell Line Culture

The human cancer cell lines were cultured in RPMI, DMEM, or Ham's F-12 medium supplemented with 10% fetal bovine serum, as described previously (Hayakawa et al., 2011).

Plasmid Transfections and Luciferase Assays

Cells were seeded into 12-well plates and after 24 hours were transfected with 200 ng of YAP luciferase reporter vector (a gift from Stefano Piccolo) and 200 ng of the M3R expression vectors (obtained from Origene) using Effectine Transfection Reagent (Qiagen). To standardize the transfection efficiency, 10 ng of Renilla luciferase vector (Promega, Madison, Wis.) was included in each sample. The total DNA concentration was kept constant through the addition of empty vector. After 24 hours, the cells were harvested in luciferase lysis buffer (Piccagene; Toyo Ink, Tokyo, Japan). The lysates were assayed for luciferase and Renilla luciferase activity using a luminometer. After standardization using the Renilla luciferase activity, luciferase activity was calculated and represented as the fold induction compared to the control.

Immunohistochemical Analysis of Human Gastric Cancer Tissue

YAP, NGF, and ChAT protein expression in human gastric cancer tissue was quantified by the proportions of positive cells and staining intensity, as described previously (Yamamoto et al., 2014) with slight modification. Briefly, proportions of YAP or NGF positive cells in cancer cells, or proportions of ChAT positive cells in stromal cells within lamina propria were scored as follows: 0, <5% positive cells; 1, 5-50% positive cells; 2, >50% positive cells. Staining intensity was scored as follows: 0, no staining; 1, faint staining; 2, moderate staining; 3, dark staining. Based on the sum of proportional scores and intensity scores (staining scores), a lesion was classified as follows: negative, when the combined score was 0 or 1; positive, when the combined score was 2 or 3; strong positive, when the combined score was 4 or 5. Two experts separately evaluated the immunohistochemical labeling in each case, and there was no disagreement. Histopathological grade of intestinal-type gastric cancer was classified into 3 groups; group 1 (well differentiated), group 2 (moderately differentiated), and group 3 (poorly differentiated). TNM class and disease stage were defined as described previously (Washington, 2010).

### REFERENCES

Aihara, T., Fujishita, T., Kanatani, K., Furutani, K., Nakamura, E., Taketo, M. M., Matsui, M., Chen, D., and Okabe, S. (2003). Impaired gastric secretion and lack of trophic responses to hypergastrinemia in M3 muscarinic receptor knockout mice. Gastroenterology 125, 1774-1784.

Aihara, T., Nakamura, Y., Taketo, M. M., Matsui, M., and Okabe, S. (2005). Cholinergically stimulated gastric acid secretion is mediated by M(3) and M(5) but not M(1) muscarinic acetylcholine receptors in mice. Am. J. Physiol. Gastrointest. Liver Physiol. 288, G1199-G1207.

Albo, D., Akay, C. L., Marshall, C. L., Wilks, J. A., Verstovsek, G., Liu, H., Agarwal, N., Berger, D. H., and Ayala, G. E. (2011). Neurogenesis in colorectal cancer is a marker of aggressive tumor behavior and poor outcomes. Cancer 117, 4834-4845.

Azzolin, L., Panciera, T., Soligo, S., Enzo, E., Bicciato, S., Dupont, S., Bresolin, S., Frasson, C., Basso, G., Guzzardo, V., et al. (2014). YAP/TAZ incorporation in the beta-catenin destruction complex orchestrates the Wnt response. Cell 158, 157-170.

Bailey, J. M., Alsina, J., Rasheed, Z. A., McAllister, F. M., Fu, Y. Y., Plentz, R., Zhang, H., Pasricha, P. J., Bardeesy, N., Matsui, W., et al. (2014). DCLK1 marks a morphologically distinct subpopulation of cells with stem cell properties in preinvasive pancreatic cancer. Gastroenterology 146, 245-256.

Bannwarth, B., and Kostine, M. (2014). Targeting nerve growth factor (NGF) for pain management: what does the future hold for NGF antagonists? Drugs 74, 619-626.

Belkind-Gerson, J Carreon-Rodriguez, A., Benedict, L. A., Steiger, C., Pieretti, A., Nagy, N., Dietrich, J., and Goldstein, A. M. (2013). Nestin-expressing cells in the gut give rise to enteric neurons and glial cells. Neurogastroenterol. Motil. 25, 61-69.e7.

Belo, A., Cheng, K., Chandi, A., Shant, J Xie, G., Khurana, S., and Raufman, J. P. (2011). Muscarinic receptor agonists stimulate human colon cancer cell migration and invasion. Am. J. Physiol. Gastrointest. Liver Physiol. 300, G749-G760.

Bezencon, C., Furholz, A., Raymond, F., Mansourian, R., Metairon, S., Le Coutre, J., and Damak, S. (2008). Murine intestinal cells expressing Trpm5 are mostly brush cells and express markers of neuronal and inflammatory cells. J. Comp. Neurol. 509, 514-525.

Brownell, I., Guevara, E., Bai, C. B., Loomis, C. A., and Joyner, A. L. (2011). Nerve-derived sonic hedgehog defines a niche for hair follicle stem cells capable of becoming epidermal stem cells. Cell Stem Cell 8, 552-565.

Cai, J., Maitra, A., Anders, R. A., Taketo, M. M., and Pan, D. (2015). beta-Catenin destruction complex-independent regulation of Hippo-YAP signaling by APC in intestinal tumorigenesis. Genes Dev. 29, 1493-1506.

Ceyhan, G. O., Schafer, K. H., Kerscher, A. G., Rauch, U., Demir, I. E., Kadihasanoglu, M., Bohm, C., Muller, M. W., Buchler, M. W., Giese, N. A., et al. (2010). Nerve growth factor and artemin are paracrine mediators of pancreatic neuropathy in pancreatic adenocarcinoma. Ann. Surg. 251, 923-931.

Chandrakesan, P., May, R., Qu, D., Weygant, N., Taylor, V. E., Li, J. D., Ali, N., Sureban, S. M., Qante, M., Wang, T. C., et al. (2015). Dclk1+small intestinal epithelial tuft cells display the hallmarks of quiescence and self-renewal. Oncotarget 6, 30876-30886.

Collins, F. (1984). An effect of nerve growth factor on the parasympathetic ciliary ganglion. J. Neurosci. 4, 1281-1288.

Collins, F., and Dawson, A. (1983). An effect of nerve growth factor on parasym- pathetic neurite outgrowth. Proc. Natl. Acad. Sci. USA 80, 2091-2094.

da Penha Berzaghi, M., Cooper, J., Castren, E., Zafra, F., Sofroniew, M., Thoenen, H., and Lindholm, D. (1993). Cholinergic regulation of brain-derived neurotrophic factor (BDNF) and nerve growth factor (NGF) but not neurotro- phin-3 (NT-3) mRNA levels in the developing rat hippocampus. J. Neurosci. 13, 3818-3826.

Dolle, L., Adriaenssens, E., El Yazidi-Belkoura, I., Le Bourhis, X., Nurcombe, V., and Hondermarck, H. (2004). Nerve growth factor receptors and signaling in breast cancer. Curr. Cancer Drug Targets 4, 463-470.

Dubeykovskaya, Z., Si, Y., Chen, X., Worthley, D. L., Renz, B. W., Urbanska, A. M., Hayakawa, Y., Xu, T., Westphalen, C. B., Dubeykovskiy, A., et al. (2016). Neural innervation stimulates splenic TFF2 to arrest myeloid cell expan- sion and cancer. Nat. Commun. 7, 10517.

Dupont, S., Morsut, L., Aragona, M., Enzo, E., Giulitti, S., Cordenonsi, M., Zanconato, F., Le Digabel, J., Forcato, M., Bicciato, S., et al. (2011). Role of YAP/TAZ in mechanotransduction. Nature 474, 179-183.

Feng, X., Degese, M. S., Iglesias-Bartolome, R., Vague, J. P., Molinolo, A. A., Rodrigues, M., Zaidi, M. R., Ksander, B. R., Merlino, G., Sodhi, A., et al. (2014). Hippo-independent activation of YAP by the GNAQ uveal melanoma oncogene through a trio-regulated rho GTPase signaling circuitry. Cancer Cell 25, 831-845.

Gautam, D., Han, S. J., Hamdan, F. F., Jeon, J., Li, B., Li, J. H., Cui, Y., Mears, D., Lu, H., Deng, C., et al. (2006). A critical role for beta cell M3 muscarinic acetylcholine receptors in regulating insulin release and blood glucose homeo- stasis in vivo. Cell Metab. 3,449-461.

Gregorieff, A., Liu, Y., Inanlou, M. R., Khomchuk, Y., and Wrana, J. L. (2015). Yap-dependent reprogramming of Lgr5(+) stem cells drives intestinal regener- ation and cancer. Nature 526, 715-718.

Gross, E. R., Gershon, M. D., Margolis, K. G., Gertsberg, Z. V., Li, Z., and Cowles, R. A. (2012). Neuronal serotonin regulates growth of the intestinal mu- cosa in mice. Gastroenterology 143, 408-417.e2.

Grundmann, D., Markwart, F., Scheller, A., Kirchhoff, F., and Schafer, K. H. (2016). Phenotype and distribution pattern of nestin-GFP-expressing cells in murine myenteric plexus. Cell Tissue Res. http://dx.doi.org/10.1007/s00441-016-2476-9.

Hanoun, M., Zhang, D., Mizoguchi, T., Pinho, S., Pierce, H., Kunisaki, Y., Lacombe, J., Armstrong, S. A., Duhrsen, U., and Frenette, P. S. (2014). Acute myelogenous leukemia-induced sympathetic neuropathy promotes malig- nancy in an altered hematopoietic stem cell niche. Cell Stem Cell 15, 365-375.

Hayakawa, Y., Ariyama, H., Stancikova, J., Sakitani, K., Asfaha, S., Renz, B. W., Dubeykovskaya, Z. A., Shibata, W., Wang, H., Westphalen, C. B., et al. (2015a). Mist1 expressing gastric stem cells maintain the normal and neoplastic gastric epithelium and are supported by a perivascular stem cell niche. Cancer Cell 28, 800-814.

Hayakawa, Y., Jin, G., Wang, H., Chen, X., Westphalen, C. B., Asfaha, S., Renz, B. W., Ariyama, H., Dubeykovskaya, Z. A., Takemoto, Y., et al. (2015b). CCK2R identifies and regulates gastric antral stem cell states and carcinogenesis. Gut 64, 544-553.

Hayakawa, Y., Sethi, N., Sepulveda, A. R., Bass, A. J., and Wang, T. C. (2016). Oesophageal adenocarcinoma and gastric cancer: should we mind the gap? Nat. Rev. Cancer 16, 305-318.

Hinoi, E., Gao, N., Jung, D. Y., Yadav, V., Yoshizawa, T., Myers, M. G., Jr., Chua, S. C., Jr., Kim, J. K., Kaestner, K. H., and Karsenty, G. (2008). The sympa- thetic tone mediates leptin's inhibition of insulin secretion by modulating os-teocalcin bioactivity. J. Cell Biol. 183, 1235-1242.

Hirota, C. L., and McKay, D. M. (2006). M3 muscarinic receptor-deficient mice retain bethanechol-mediated intestinal ion transport and are more sensitive to colitis. Can. J. Physiol. Pharmacol. 84, 1153-1161.

Howard, L., Wyatt, S., Nagappan, G., and Davies, A. M. (2013). ProNGF promotes neurite growth from a subset of NGF-dependent neurons by a p75NTR-dependent mechanism. Development 140, 2108-2117.

Imajo, M., Ebisuya, M., and Nishida, E. (2015). Dual role of YAP and TAZ in renewal of the intestinal epithelium. Nat. Cell Biol. 17, 7-19.

Jiao, S., Wang, H., Shi, Z., Dong, A., Zhang, W., Song, X., He, F., Wang, Y., Zhang, Z., Wang, W., et al. (2014). A peptide mimicking VGLL4 function acts as a YAP antagonist therapy against gastric cancer. Cancer Cell 25, 166-180.

Kabouridis, P. S., Lasrado, R., McCallum, S., Chng, S. H., Snippert, H. J., Clevers, H., Pettersson, S., and Pachnis, V. (2015). Microbiota controls the homeostasis of glial cells in the gut lamina propria. Neuron 85, 289-295.

Katayama, Y., Battista, M., Kao, W. M., Hidalgo, A., Peired, A. J., Thomas, S. A., and Frenette, P. S. (2006). Signals from the sympathetic nervous system regu- late hematopoietic stem cell egress from bone marrow. Cell 124, 407-421.

Kniewallner, K. M., Grimm, N., and Humpel, C. (2014). Platelet-derived nerve growth factor supports the survival of cholinergic neurons in organotypic rat brain slices. Neurosci. Lett. 574, 64-69.

Kodaira, M., Kajimura, M., Takeuchi, K., Lin, S., Hanai, H., and Kaneko, E. (1999). Functional muscarinic m3 receptor expressed in gastric cancer cells stimulates tyrosine phosphorylation and MAP kinase. J. Gastroenterol. 34, 163-171.

Lapchak, P. A., Araujo, D. M., and Hefti, F. (1993). Cholinergic regulation of hippocampal brain-derived neurotrophic factor mRNA expression: evidence from lesion and chronic cholinergic drug treatment studies. Neuroscience 52, 575-585.

Leushacke, M., Ng, A., Galle, J., Loeffler, M., and Barker, N. (2013). Lgr5+ gastric stem cells divide symmetrically to effect epithelial homeostasis in the pylorus. Cell Rep. 5, 349-356.

Li, X., Nadauld, L., Ootani, A., Corney, D. C., Pai, R. K., Gevaert, O., Cantrell, M. A., Rack, P. G., Neal, J. T., Chan, C. W., et al. (2014). Oncogenic transforma- tion of diverse gastrointestinal tissues in primary organoid culture. Nat. Med. 20, 769-777.

Lundgren, O., Jodal, M., Jansson, M., Ryberg, A. T., and Svensson, L. (2011). Intestinal epithelial stem/progenitor cells are controlled by mucosal afferent nerves. PLoS One 6, e16295.

Magnon, C., Hall, S. J., Lin, J., Xue, X., Gerber, L., Freedland, S. J., and Frenette, P. S. (2013). Autonomic nerve development contributes to prostate cancer progression. Science 341, 1236361.

Mahmoud, A. I., O'Meara, C. C., Gemberling, M., Zhao, L., Bryant, D. M., Zheng, R., Gannon, J. B., Cai, L., Choi, W. Y., Egnaczyk, G. F., et al. (2015). Nerves regu- late cardiomyocyte proliferation and heart regeneration. Dev. Cell 34, 387-399.

Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Mesenchymal and haematopoietic stem cells form a unique bone marrow niche. Nature 466, 829-834.

Mignone, J. L., Kukekov, V., Chiang, A. S., Steindler, D., and Enikolopov, G. (2004). Neural stem and progenitor cells in nestin-GFP transgenic mice. J. Comp. Neurol. 469, 311-324.

Neal, K. B., and Bornstein, J. C. (2007). Mapping 5-HT inputs to enteric neurons of the Guinea-pig small intestine. Neuroscience 145, 556-567.

Okumura, T., Ericksen, R. E., Takaishi, S., Wang, S. S., Dubeykovskiy, Z., Shibata, W., Betz, K. S., Muthupalani, S., Rogers, A. B., Fox, J. G., et al. (2010). K-ras mutation targeted to gastric tissue progenitor cells results in chronic inflammation, an altered microenvironment, and progression to intra-epithelial neoplasia. Cancer Res. 70, 8435-8445.

Peterson, S. C., Eberl, M., Vagnozzi, A. N., Belkadi, A., Veniaminova, N. A., Verhaegen, M. E., Bichakjian, C. K., Ward, N. L., Dlugosz, A. A., and Wong, S. Y. (2015). Basal cell carcinoma preferentially arises from stem cells within hair follicle and mechanosensory niches. Cell Stem Cell 16, 400-412.

Quante, M., Bhagat, G., Abrams, J. A., Marache, F., Good, P., Lee, M. D., Lee, Y., Friedman, R., Asfaha, S., Dubeykovskaya, Z., et al. (2012). Bile acid and inflammation activate gastric cardia stem cells in a mouse model of Barrett-like metaplasia. Cancer Cell 21, 36-51.

Raufman, J. P., Samimi, R., Shah, N., Khurana, S., Shant, J., Drachenberg, C., Xie, G., Wess, J., and Cheng, K. (2008). Genetic ablation of M3 muscarinic receptors attenuates murine colon epithelial cell proliferation and neoplasia. Cancer Res. 68, 3573-3578.

Rosenbluh, J., Nijhawan, D., Cox, A. G., Li, X., Neal, J. T., Schafer, E. J., Zack, T. I., Wang, X., Tsherniak, A., Schinzel, A. C., et al. (2012). beta-Catenin-driven cancers require a YAP1 transcriptional complex for survival and tumorigen- esis. Cell 151, 1457-1473.

Schutz, B., Jurastow, I., Bader, S., Ringer, C., von Engelhardt, J., Chubanov, V., Gudermann, T., Diener, M., Kummer, W., Krasteva-Christ, G., et al. (2015). Chemical coding and chemosensory properties of cholinergic brush cells in the mouse gastrointestinal and biliary tract. Front. Physiol. 6, 87.

Shi, G., Zhu, L., Sun, Y., Bettencourt, R., Damsz, B., Hruban, R. H., and Konieczny, S. F. (2009). Loss of the acinar-restricted transcription factor Mist1 ac- celerates Kras-induced pancreatic intraepithelial neoplasia. Gastroenterology 136, 1368-1378.

Song, P., Sekhon, H. S., Lu, A., Arredondo, J., Sauer, D., Gravett, C., Mark, G. P., Grando, S. A., and Spindel, E. R. (2007). M3 muscarinic receptor antago- nists inhibit small cell lung carcinoma growth and mitogen-activated protein kinase phosphorylation induced by acetylcholine secretion. Cancer Res. 67, 3936-3944.

Stopczynski, R. E., Normolle, D. P., Hartman, D. J., Ying, H., DeBerry, J. J., Bielefeldt, K., Rhim, A. D., DePinho, R. A., Albers, K. M., and Davis, B. M. (2014). Neuroplastic changes occur early in the development of pancreatic ductal adenocarcinoma. Cancer Res. 74, 1718-1727.

Tallini, Y. N., Shui, B., Greene, K. S., Deng, K. Y., Doran, R., Fisher, P. J., Zipfel, W., and Kotlikoff, M. I. (2006). BAC transgenic mice express enhanced green fluorescent protein in central and peripheral cholinergic neurons. Physiol. Genomics 27,391-397.

Tutton, P. J., and Barkla, D. H. (1986). Serotonin receptors influencing cell pro- liferation in the jejunal crypt epithelium and in colonic adenocarcinomas. Anticancer Res. 6, 1123-1126.

Tutton, P. J., and Helme, R. D. (1973). Proceedings: the role of catecholamines in the regulation of crypt cell proliferation. I. Adrenergic stimulation and blockade. J. Anat. 116, 467-468.

Vaishnavi, A., Le, A. T., and Doebele, R. C. (2015). TRKing down an old onco- gene in a new era of targeted therapy. Cancer Discov. 5, 25-34.

Venkatesh, H. S., Johung, T. B., Caretti, V., Noll, A., Tang, Y., Nagaraja, S., Gibson, E. M., Mount, C. W., Polepalli, J., Mitra, S. S., et al. (2015). Neuronal activity promotes glioma growth through Neuroligin-3 secretion. Cell 161, 803-816.

von Moltke, J., Ji, M., Liang, H. E., and Locksley, R. M. (2016). Tuft-cell-derived IL-25 regulates an intestinal ILC2-epithelial response circuit. Nature 529, 221-225.

Weeraratna, A. T., Dalrymple, S. L., Lamb, J. C., Denmeade, S. R., Miknyoczki, S., Dionne, C. A., and Isaacs, J. T. (2001). Pan-trk inhibition decreases metastasis and enhances host survival in experimental models as a result of its selective in- duction of apoptosis of prostate cancer cells. Clin. Cancer Res. 7, 2237-2245.

Westphalen, C. B., Asfaha, S., Hayakawa, Y., Takemoto, Y., Lukin, D. J., Nuber, A. H., Brandtner, A., Setlik, W., Remotti, H., Muley, A., et al. (2014). Long-lived intestinal tuft cells serve as colon cancer-initiating cells. J. Clin. Invest. 124, 1283-1295.

Yu, F. X., Zhao, B., Panupinthu, N., Jewell, J. L., Lian, I., Wang, L. H., Zhao, J., Yuan, H., Tumaneng, K., Li, H., et al. (2012). Regulation of the Hippo-YAP pathway by G-protein-coupled receptor signaling. Cell 150, 780-791.

Yu, F. X., Luo, J., Mo, J. S., Liu, G., Kim, Y. C., Meng, Z., Zhao, L., Peyman, G., Ouyang, H., Jiang, W., et al. (2014). Mutant Gq/11 promote uveal melanoma tumorigenesis by activating YAP. Cancer Cell 25, 822-830.

Zhao, C. M., Hayakawa, Y., Kodama, Y., Muthupalani, S., Westphalen, C. B., Andersen, G. T., Flatberg, A., Johannessen, H., Friedman, R. A., Renz, B. W., et al. (2014). Denervation suppresses gastric tumorigenesis. Sci. Transl. Med. 6, 250ra115.

Hayakawa, Y., Hirata, Y., Nakagawa, H., Sakamoto, K., Hikiba, Y., Kinoshita, H., Nakata, W., Takahashi, R., Tateishi, K., Tada, M., et al. (2011). Apoptosis signal-regulating kinase 1 and cyclin D1 compose a positive feedback loop contributing to tumor growth in gastric cancer. Proc Natl Acad Sci U S A 108, 780-785.

Washington, K. (2010). 7^{th} edition of the AJCC cancer staging manual: stomach. Ann Surg Oncol 17, 3077-3079.

Yamamoto, K., Tateishi, K., Kudo, Y., Sato, T., Yamamoto, S., Miyabayashi, K., Matsusaka, K., Asaoka, Y., Ijichi, H., Hirata, Y., et al. (2014). Loss of histone demethylase KDM6B enhances aggressiveness of pancreatic cancer through downregulation of C/EBPalpha. Carcinogenesis 35, 2404-2414.

### EXAMPLE 4

### Cholinergic Agonism in the Treatment and Mitigation of Intestinal Injury

As described herein, the gastrointestinal tract is strongly regulated and innervated by cholinergic nerves. Cholinergic nerves in the stomach and intestines regulate directly stem cells. As described herein (Zhao CM et al) showed that this receptor was the muscarinic-3 receptor (M3R). Inhibition of muscarinic receptors is therefore useful in inhibiting tumorigenesis.

The corollary to tumorigenesis was also considered and the stimulation of muscarinic receptors could be useful in stimulating gut stem cells, and thus improving regeneration following acute or chronic injury.

Broad muscarinic cholinergic agonists, such as pilocarpine or bethanechol, can be used to simulate intestinal stem cells under conditions of homeostasis or following injury. Following the observations described herein with gastric stem cells, it is shown that cholinergic agonists could stimulate intestinal or colonic organoids as well.

There are two sources of acetylcholine in the gut-nerves and tuft cells-since both express the gene ChAT. Knockout of tuft cells (using Dclk1-CreERT; R26-DTR) impaired intestinal regeneration following radiation or DSS colitis, in part because of loss of ChAT and acetylcholine production (Westphalen CB et al, JCI 2014). However, replacement of signaling by giving the mice bethanechol in the drinking water was able to improve intestinal regeneration, with reduction in ulcer length and improved proliferation. Loss of the M3R in the intestine (Villin-Cre; Chrm3 f/f) led to worse colitis and reduced regeneration following DSS, indicating that regeneration requires muscarinic receptor signaling in the epithelium.

Bethanechol, an FDA approved drug for bladder dysfunction, can be used in healing the gut after significant injury such as after radiation or inflammatory bowel disease. The effects of bethanechol were tested on response to lethal irradiation. Mice were given 15 Gy abdominal irradiation (AIR), and bethanechol was given immediately before or after the radiation. The results initially suggested that when administered as a single agent, there was no significant overall impact on survival. However, when the effects on individual segments of the bowel were studied, there was a remarkable effect on the colon. At day +4 after radiation, the histopathologic score for the colon was >65% improved in bethanechol-treated mice compared to the controls, in both the pre- and post-radiation treated mice. In both case, the colon was almost normal appearing after radiation.

Significant improvement has also been seen with bethanechol-treated of DSS colitis. Thus, bethanechol and related cholinergic agonists can be used for healing the injured. For example, bethanechol or other muscarinic cholinergic agonists can be used for treatment of radiation- or chemotherapy-induced proctitis/colitis. This is a major and common complication of current cancer treatments, and can be markedly mitigated by bethanechol given by enema or orally. Bethanechol or other muscarinic cholinergic agonists can also be used for treatment of ulcerative colitis, for example, given as an enema, it could improve healing. Bethanechol or other muscarinic cholinergic agonists, alone or in combination with other agents, can also be used for mitigation of the RIGS (radiation induced gastrointestinal syndrome).

What is claimed:

1. A method for treating gastric or colon cancer in a subject in need thereof, the method comprising administering to the subject a cholinergic antagonist, a Botulinum toxin, a NGF inhibitior, a TRK inhibitor, performing surgical denervation on the subject, or a combination thereof.

2. A method for reducing or inhibiting proliferation of gastric or colon tumor cells, the method comprising administering a cholinergic antagonist, a Botulinum toxin, a NGF inhibitior, a TRK inhibitor, performing surgical denervation, or a combination thereof.

3. A method for inhibiting stem cell growth, the method comprising administering a cholinergic antagonist, a Botulinum toxin, a NGF inhibitior, a TRK inhibitor, performing surgical denervation, or a combination thereof.

4. A method for stimulating growth of stem cells, the method comprising administering a cholinergic agonist.

5. A method for stimulating regeneration of the colon or stomach in a subject in need thereof, the method comprising administering to the subject a cholinergic agonist.

6. A method for treating colitis or gastritis in a subject in need thereof, the method comprising administering to the subject a cholinergic agonist.

7. A method for treating radiation-induced gastrointestinal syndrome (RIGS) in a subject in need thereof, the method comprising administering to the subject a cholinergic agonist.

8. The method of claim 1, wherein the surgical denervation is a vagotomy, a bilateral vagotomy with pyloroplasty, or a unilateral vagotomy.

9. The method of claim 1, wherein the Botulinum toxin inhibits local signaling from the vagus nerve.

10. The method of claim 1, wherein the cholinergic antagonist is a muscarinic receptor antagonist.

11. The method of claim 1, wherein the cholinergic antagonist is a M3 receptor antagonist.

12. The method of claim 1, wherein the cholinergic antagonist is darifenacin, scopolamine, amitriptyline, or a tricyclic antidepressant.

13. The method of claim 1, wherein the TRK inhibitor is PLX7486.

14. The method of claim 1, further comprising administering a cytotoxic therapy.

15. The method of claim 14, wherein the cholinergic antagonist, Botulinum toxin, or surgical denervation is administered or performed before, during, or after the administration of the cytotoxic therapy.

16. The method of claim 14, wherein the cytotoxic therapy is radiotherapy or chemotherapy.

17. The method of claim 1, further comprising performing an endoscopic resection surgery or a gastrectomy surgery.

18. The method of claim 17, wherein the cholinergic antagonist, Botulinum toxin, or surgical denervation is administered or performed before, during, or after the endoscopic resection surgery or gastrectomy surgery is performed.

19. The method of claim 3, wherein the stem cells are cancer stem cells.

20. The method of claim 19, wherein the cancer stem cells are gastric cancer stem cells, or colon cancer stem cells.

21. The method of claim 3, wherein the stem cells express Lgr5, M3 receptor, or a combination thereof.

22. The method of claim 4, wherein the stem cells are gastric stem cells, or colon stem cells.

23. The method of claim 5, wherein the subject has colitis, gastritis, radiation-induced proctitis, radiation-induced colitis, chemotherapy-induced proctitis, chemotherapy-induced colitis, radiation induced gastrointestinal syndrome (RIGS), inflammatory bowel disease, or ulcerative colitis.

24. The method of claim 5, wherein the cholinergic agonist is pilocarpine or bethanechol.


SEQUENCE LISTING (24 sequences)

SEQ ID NO: 1 - DNA, 18 bp, Artificial Sequence
  Description of Artificial Sequence Synthetic primer
tccaacctcagcgtcttc

SEQ ID NO: 2 - DNA, 18 bp, Artificial Sequence
  Description of Artificial Sequence Synthetic primer
tgggaatgtgtgtcaaag

SEQ ID NO: 3 - DNA, 23 bp, Artificial Sequence
  Description of Artificial Sequence Synthetic primer
cacatattgcttcaatgcctcag

SEQ ID NO: 4 - DNA, 24 bp, Artificial Sequence
  Description of Artificial Sequence Synthetic primer
ccatcacggttgacaatagttatg

SEQ ID NO: 5 - DNA, 20 bp, Artificial Sequence
  Description of Artificial Sequence Synthetic primer
actgaccgacgattccatgt

SEQ ID NO: 6 - DNA, 20 bp, Artificial Sequence
  Description of Artificial Sequence Synthetic primer
tgcatctctctctggagctg

SEQ ID NO: 7 - DNA, 20 bp, Artificial Sequence
  Description of Artificial Sequence Synthetic primer
agagctcctcgagctgtttg

SEQ ID NO: 8 - DNA, 19 bp, Artificial Sequence
  Description of Artificial Sequence Synthetic primer
tgaagttcacgttgagggg

SEQ ID NO: 9 - DNA, 20 bp, Artificial Sequence
  Description of Artificial Sequence Synthetic primer
tcctctccaaaatgccagag

SEQ ID NO: 10 - DNA, 19 bp, Artificial Sequence
  Description of Artificial Sequence Synthetic primer
gggtgggttggaaatgaac

SEQ ID NO: 11 - DNA, 20 bp, Artificial Sequence
  Description of Artificial Sequence Synthetic primer
aggaagctggcagaccagta

SEQ ID NO: 12 - DNA, 20 bp, Artificial Sequence
  Description of Artificial Sequence Synthetic primer
tccacgaagggtctcttctc

SEQ ID NO: 13 - DNA, 25 bp, Artificial Sequence
  Description of Artificial Sequence Synthetic primer
cagaagtggtgatcaagatgcctat

SEQ ID NO: 14 - DNA, 20 bp, Artificial Sequence
  Description of Artificial Sequence Synthetic primer
gagcttttgggaggctgctt

SEQ ID NO: 15 - DNA, 23 bp, Artificial Sequence
  Description of Artificial Sequence Synthetic primer
tggagcacaacaagatccagaat

SEQ ID NO: 16 - DNA, 19 bp, Artificial Sequence
  Description of Artificial Sequence Synthetic primer
cccctgaacgcagttttca

SEQ ID NO: 17 - DNA, 21 bp, Artificial Sequence
  Description of Artificial Sequence Synthetic primer
ccagttggtgtgttcttcctt

SEQ ID NO: 18 - DNA, 20 bp, Artificial Sequence
  Description of Artificial Sequence Synthetic primer
aggaagagctgatgttggga

SEQ ID NO: 19 - DNA, 22 bp, Artificial Sequence
  Description of Artificial Sequence Synthetic primer
gtgactgccatcgagatcgtac

SEQ ID NO: 20 - DNA, 20 bp, Artificial Sequence
  Description of Artificial Sequence Synthetic primer
caaactttcgggccacattg

SEQ ID NO: 21 - DNA, 19 bp, Artificial Sequence
  Description of Artificial Sequence Synthetic primer
ggcccagagagaacggaac

SEQ ID NO: 22 - DNA, 20 bp, Artificial Sequence
  Description of Artificial Sequence Synthetic primer
ttcccgttgttgaggtgctt

SEQ ID NO: 23 - DNA, 23 bp, Artificial Sequence
  Description of Artificial Sequence Synthetic primer
tcattgtcataccaggaaatgag

SEQ ID NO: 24 - DNA, 23 bp, Artificial Sequence
  Description of Artificial Sequence Synthetic primer
agaaacctgccaagtatgatgac